(19) United States
(12) Patent Application Publication
(10) Pub. No.: US 2025/0270253 A1
(43) Pub. Date: Aug. 28, 2025

(54) SITE-SELECTIVE MODIFICATION OF PROTEINS

(71) Applicants: DANMARKS TEKNISKE UNIVERSITET, Kongens Lyngby (DK); UNIVERSITY OF COPENHAGEN, Copenhagen K (DK); Rigshospitalet, København Ø (DK)

(72) Inventors: Sanne SCHOFFELEN, Kongens Lyngby (DK); Knud Jørgen JENSEN, Copenhagen Ø (DK); Kasper Kildegaard SØRENSEN, Smørum (DK); Mikkel B. THYGESEN, Fredensborg (DK); Christian KOFOED, Princeton, NJ (US)

(21) Appl. No.: 18/034,407

(22) PCT Filed: Oct. 29, 2021
(86) PCT No.: PCT/EP2021/080219

§ 371 Date: Apr. 28, 2023

(30) Foreign Application Priority Data
Oct. 30, 2020 (EP) 20204915.1

(51) Int. Cl. C07K 1/107; C07K 1/13

(52) U.S. Cl. CPC C07K 1/1077; C07K 1/13

(57) ABSTRACT

The present invention concerns a method for site-selective modification of a target protein or peptide by use of an acylation tag comprising a single lysine residue and at least three histidine residues. Upon contact with an acylating reagent, the target protein or peptide becomes modified at the ε-amine of the lysine residue of the acylation tag.

### FIELD OF THE INVENTION

The present invention relates to a method for site-specifically modifying a target protein or peptide and thereby allows for the conjugation of selected entities to a protein or peptide of interest in order to improve or manipulate the properties of the protein or peptide (such as biopharmaceuticals) and/or to facilitate detailed studies of its structure and function.

### BACKGROUND OF THE INVENTION

The chemical modifications of proteins to introduce for example fluorophores, half-life extending moieties, and binders, such as biotin, is of great importance in chemical biology and for biotechnology and biopharmaceuticals.

Many pharmaceutical companies have been refocusing their pipeline towards biological medicines (mainly monoclonal antibodies) because of the high specificity and safety. A next generation of biological medicines are the antibody drug conjugates (ADCs), which efficiently deliver the payload to the target limiting the off-target effects. Site-specific modifications to improve the properties of these therapeutic proteins are highly attractive.

Because proteins are biomolecules with a tertiary structure, the labeling conditions used to create such conjugates have to be mild and reactions should take place in aqueous solution.

The introduction of novel chemical groups often relies on modification of canonical amino acids, most typically cysteine (Cys) and lysine (Lys) residues. However, these reactions are often not very site-selective and lack protein-specificity when performed in complex mixtures.

The oldest and most straightforward method for labeling proteins is via the primary amino groups on lysine residues and at the N-terminus. Labeling of amines is often carried out using NHS esters, but in a highly unspecific manner, since in general, multiple accessible lysines having reactive amines are present on the protein surface, resulting in efficient labeling, but inevitably leading to heterogeneous mixtures.

Lysine reactivity is related to the pKa of the ε-amine. Notably, the pKa values of lysines within the same protein can differ by as much as 5 units 1, shifts that arise from differences in the chemical microenvironment of the individual residues. Some strategies have therefore been to target the most reactive lysine available, by using substoichiometric to equivalent ratios of a given reagent to the protein of interest. This has been achieved using NHS esters² and methyl 2-(sulfonylmethyl)acrylate³. Although promising, the use of such kinetically controlled labeling is still not widespread, and suffers from requiring cumbersome optimization of conditions in some cases², in addition to being theoretically incompatible with enzymes that rely on catalytic lysines or cysteines for their mechanism of action³.

Selectivity can also be achieved using NHS esters carrying a nitrilotriacetic acid (NTA) chelator to guide labeling of His-tagged protein^{4,5}. Upon binding to an appropriate metal ion in a tripartite complex, the NTA NHS ester and His-tagged protein are brought in close range of each other. This complexation will guide labeling of the His-tagged protein within the radius of the bound NHS ester. However, this proximity-based labeling method is limited by several factors. Firstly, any reactive amine will in theory be prone to undergo modification. Secondly, before complexation the NHS ester is still free to undergo reaction with any available amine. Thirdly, not all proteins tolerate metal ions, but can undergo inactivation⁶ and protein aggregation^{7,8}. Lastly, the NTA carrying NHS ester reagent is not commercially available.

Common for all known lysine-labeling strategies is that they target the most reactive native amine available, which is typically not known a priori. Computational methods to predict residue pKa can in theory assist in the prediction of which lysine would be targeted for modification, however such tools rely on 3D structures, which are not always available, and can have large error margins, so outcomes are attached with large uncertainties. As such selective labeling of native lysines remains an empirical chemistry that is not easily transferable across protein species.

Accordingly, the present invention addresses the need for methods and tools for site-specific modification of target proteins or peptides and thereby allows for the conjugation of selected entities to any given protein or peptide of interest.

### SUMMARY OF THE INVENTION

In a first aspect, the present invention provides a method for site-selective modification of a target protein or peptide comprising the steps of:
a. providing a target protein or peptide wherein the amino acid sequence of said protein or peptide comprises an acylation tag,
b. contacting the target protein or peptide from step (a) with an acylating reagent to form a modified target protein or peptide,

wherein said acylation tag comprises a single lysine residue and at least three histidine residues, and wherein the target protein or peptide upon contact with the acylating reagent becomes modified at the ε-amine of the lysine residue of the acylation tag.

A second aspect of the invention provides an acylated protein or peptide comprising an acylation tag, wherein said acylation tag comprises or consists of an amino acid sequence selected from:

i.
(His)ₐ-(X¹)_{b}-Lys,
ii.
Lys-(X¹)_{b}-(His)ₐ,
iii.
(His)ₐ-(X¹)_{b}-Lys-(X²)c-(His)ₐ
and
iv.
(His)_{d}-(X²)_{c}-Lys-(X¹)_{b}-(His)ₐ

wherein a≥3, b=0-3, c=0-3, and d 1, and wherein X¹ and X² each are one or more identical or different amino acids but not lysine, and wherein the ε-amine of Lys in said acylation tag is acylated.

A third aspect of the invention provides a composition/kit comprising an acylated protein or peptide according to the second aspect.

A fourth aspect of the invention provides a kit for modifying a target protein or peptide, said kit comprising:
a. a target protein or peptide, or a nucleic acid sequence encoding said target protein or peptide, wherein said target protein or peptide comprises an acylation tag, wherein said acylation tag comprises or consists of an amino acid sequence selected from:

i.
(His)ₐ-(X¹)_{b}-Lys,
ii.
Lys-(X¹)_{b}-(His)ₐ,
iii.
(His)ₐ-(X¹)_{b}-Lys-(X²)c-(His)ₐ
and
iv.
(His)_{d}-(X²)_{c}-Lys-(X¹)_{b}-(His)ₐ

wherein a≥3, b=0-3, c=0-3, and d 1, and wherein X¹ and X² each are one or more identical or different amino acids but not lysine, and
b. an acylating reagent.

A fifth aspect of the invention concerns the use of an acylation tag for site-selective modification of a target protein or peptide, wherein said acylation tag comprises an amino acid sequence located internally or at the C-terminus of said target protein or peptide, wherein said amino acid sequence of said acylation tag comprises a single lysine residue and at least three histidine residues; and wherein the ε-amine of the lysine residue of the acylation tag is capable of being acylated upon contact with an acylating reagent.

### DESCRIPTION OF THE INVENTION

### Brief Description of the Figures

FIG. 1: (A) Prior art: Non-selective labeling of a protein or peptide [POI] using N-succinimide ester derivatives, by modifying native amines of lysine residues. (B) Present invention: Site-selective labeling of a protein or peptide [POI] comprising a Lys-His tag [KHHHHHH], by modifying the ε-amine of the lysine residue within the tag, using phenyl ester derivatives. In both (A) and (B), the reaction leads to labeling of the protein or peptide [POI] with a chosen molecule depicted by the star. Shown in the figure is the KH₆ tag version, although various other sequence combinations disclosed herein are suitable.

FIG. 2: (A) Representations of the three proteins Small Ubiquitin-like Modifier (SUMO), superfolder Green Fluorescent Protein (sfGFP), and Maltose Binding Protein (MBP). Lysine residues and the N-terminal of each protein are shown as spheres. Terminals as indicated. Crystal structures (protein data bank accession code) used: 1AR5 (SUMO), 2B3P (sfGFP), and 1ANF (MBP). (B) On the left, a representation of sfGFP, indicating lysine residues as sticks. Arrows indicate β-strands 10 and 11 in addition to the connecting loop. On the right, mapped B-factor to display the relative vibrational motion of the backbone in the crystal structure.

FIG. 3: Reaction scheme for the two-step fluorescent labeling of a protein or peptide [POI] comprising a Lys-His tag [KH₆]: (1) acylation reaction with compound 1, (2) conjugation with compound 2 that is an alkyne-functionalized cyanine dye.

FIG. 4: Images of polyacrylamide gels showing proteins in fractions from the cleared lysate (CL), IMAC (IF) and size-exclusion chromatography (SF) obtained during purification of Lys-His tagged proteins. (A) shows the affinity purification of KH₆ and H3KH₆ versions of the Lys-His tagged proteins by IMAC (compare CL with IF). (B) shows the affinity purification of H3KH₆ tagged versions of SUMO, sfGFP, and MBP by IMAC that selectively enriches for each protein.

FIG. 5: Images of polyacrylamide gels showing fluorescence labeled of sfGFP, SUMO, and MBP comprising a KH₆ or H3KH₆ C-terminal tag, compared to tag-free versions. Half of the samples were treated with acylating reagent 1, before conjugation with alkyne-functionalized cyanine fluorophore 2. The top row images show gels of Coomassie Blue stained purified proteins, while the bottom row show fluorescence images of the same gels after treatment using fluorophore 2.

FIG. 6: Images of polyacrylamide gels showing Ni-NTA affinity capture of tagged sfGFP. Purified sfGFP, comprising the indicated tag, was incubated with Ni-NTA resin, the resin washed with buffer, and finally any captured tagged sfGFP eluted with buffer containing imidazole. The images show the applied fraction (A), and the eluted fraction (E).

FIG. 7: ESI-TOF LC-MS spectra of differently tagged sfGFP. Deconvoluted mass spectra (units in daltons). SM=starting material (non-modified protein), 1 mod.=mono-functionalized protein, 2 mod.=di-functionalized protein. Shown are the results of three independent experiments (spectra 1-3), where protein (29 μM) was reacted with 40 equivalents of 4-methoxyphenyl ester 1 in aqueous 50 mM NaH₂PO₄, 150 mM NaCl, 1 mM EDTA, 8% DMSO (pH 7.5) at 4° C. for 16 hr. The reaction with sfGFP(H₃PKH₃) contained the already modified adduct *SM, which seemingly only underwent background acylation to produce *1 mod. as shown in the respective insert of reaction 1.

FIG. 8: Images of polyacrylamide gels showing fluorescence labeled of internally tagged sfGPF, compared to tag-free sfGFP. Half of the samples were treated with acylating reagent 1, before conjugation with alkyne-functionalized cyanine fluorophore 2. The top row images show gels of Coomassie Blue stained purified proteins, while bottom row shows fluorescence images of the same gels after treatment using fluorophore 2.

FIG. 9: Mass spectrometry spectra of different versions of the Lys-His tag inserted in loop region of sfGPF. SM=starting material (non-modified protein), P1=mono-functionalized protein, P2=di-functionalized protein.

FIG. 10: Mass spectrometry spectra of different versions of the Lys-His tag inserted in a loop region of sfGPF. 5 mM EDTA was added to the acylation reaction to improve efficiency. SM=starting material (non-modified protein), P1=mono-functionalized protein, P2=di-functionalized protein.

FIG. 11: Images of biotin labeled sfGFP captured on resin beads. Purified protein was mock treated or incubated with either biotinylating reagent 5 or 6a. Affinity capture was then tested by incubation of the treated protein with streptavidin immobilized on resin.

FIG. 12: Mass spectrometry spectra of sfGFP, sfGFP-KH₆, and sfGFP-H3KH₆ reacted with either 5, 6a, 3, 4a, or 4b. 0 mod.=starting material, non-modified protein, 1, 2, 3, 4, and 5 mod.=mono-, di-, tri-, tetra-, penta-functionalized protein, respectively.

FIG. 13: Images of Western blot and Coomassie-stained polyacrylamide gels of SUMO and MBP reacted with biotinylation reagents 6a and 6b. Lane 1: tag-free protein, lane 2: KH₆-tagged protein, lane 3: H₃KH₆-tagged protein.

FIG. 14: Western blot image (left) and Coomassie-stained gel image (right) of polyacrylamide gels both containing an aliquot of the same sample comprising a solution of Rituximab-H₃KH₆ supplemented with five untagged proteins (a: conalbumin, b: BSA, c: ovalbumin, d: aldolase, and e: lysozyme) reacted with biotinylation reagent 6a. Hc and Lc indicate the heavy chain and light chain of the antibody, respectively.

FIG. 15: Deconvoluted ESI-TOF spectra of the heavy and light chain of Rituximab-KH₆ treated without (left) and with (right) acylation reagent 1.

FIG. 16: Deconvoluted ESI-TOF spectra of the heavy and light chain of Rituximab-H₃KH₆ treated without (left) and with (right) acylation reagent 1.

FIG. 17: Deconvoluted MS spectra of the heavy chain of Rituximab-KHn with n=1-6. Top row: no acylation reagent; Bottom row: with acylation reagent 1 (0.9 mM). Expected mass shift when acylated is 57 Da. SM: starting material, P1: monofunctionalized product.

FIG. 18: Deconvoluted MS spectra of Rituximab-KH₄ heavy and light chain—expected mass shift is 57 Da (for both reagents). SM: starting material, SM-ox: oxidized starting material, P1: monofunctionalized product.

FIG. 19: Mass spectrometric analysis of acylated sfGFP-H₃KH₆ before and after thrombin treatment. Thrombin cleaves sfGFP-H₃KH₆ between arginine and glycine (amino acid residues 242 and 243 in SEQ ID NO. 40) which is just before the H₃KH₆ tag. (A): MS spectrum of sfGFP-H₃KH₆ reacted with acylating agent 1 before thrombin treatment. The same spectrum is displayed over a broad mass range (500-1500 m/z) and after zooming in on the 31+ charged peak. The latter picture shows the relative abundance of starting material (SM), monoacylated product (P1) and diacylated product (P2). (B): LC trace (UV, 180-900 nm) of the sample after thrombin treatment. (C) MS spectra of the cleaved Lys-His tag peptide after thrombin treatment (residues 243-254), showing that the tag is mainly monoacylated. (D) MS spectra of the truncated protein (sfGPF Δ243-254), showing that the majority of the truncated protein is unmodified. Note that the relative amounts of unmodified Lys-His tag peptide and of monoacylated truncated protein correspond very well with the relative abundance of the SM and P2 species in (A), respectively.

FIG. 20: pH screen of the modification of sfGFP-H₃KH₆ with acylating agent 1. MS spectra were acquired after the protein (29 μM) was reacted with 40 equivalents of reagent 1 at the indicated buffer pH. The mass region spanning one of the most abundant peaks (31+) of the multiple-charged envelope, is displayed for each spectrum. Peaks corresponding with starting material (SM), mono-functionalized product (P1) and di-functionalized product (P2) are indicated.

FIG. 21: Enzymatic cleavage study of acylated Lys-His tagged Beltide peptide. (A) LC-MS data for isolated, mono-2-azidoacetylated H-Beltide-H₃KH₆-OH. Reaction conditions: Peptide (100 μM), ester 1 (20 equiv.), PBS buffer, pH 7.5, 4° C., 16 h. Insert chromatogram: Crude reaction mixture. (B) Structure of mono-acylated H-Beltide-H₃KH₆-OH at the Lys-His tag and the C-terminal V8 cleavage site at aspartic acid (D) and glutamic acid (E) indicated. Four fragments were observed in the enzymatic study. (C) HPLC (UV) and ion-extracted chromatograms of mono-2-azidoacetylated H-Beltide-H₃KH₆-OH treated with V8 protease. (D) MS spectra of the four fragments. The primary site of acylation is the lysine in the Lys-His tag of fragment 2. No acylation was observed on the N-terminus (fragment 4) or other lysine residues.

### ABBREVIATIONS, TERMS, AND DEFINITIONS

N-terminus and C-terminus refer to the amino acid located at the extreme amino and carboxyl ends of a protein or peptide amino acid sequence, respectively. Internal location refers to any amino acid that forms part of the amino acid sequence of a protein or peptide except the amino acid located at the extreme amino and carboxyl ends.

Amino acid abbreviation: standard three letter or one letter amino acid abbreviations are used herein to represent amino acid residues that constitute the peptides and polypeptides of the invention, e.g. Lysine=Lys=K; Histidine=His=H. Strings of amino acid abbreviations are used to represent peptides and polypeptides, with the N-terminus indicated on the left; the sequence is written from the N-terminus to the C-terminus.

Target proteins or peptides of the present invention comprise an acylation tag, which upon contact with an acylating reagent becomes modified at the ε-amine of the acylation tag lysine residue. The method of the present invention of site-specific modifying proteins or peptides is not limited to specific protein or peptide classes, but broadly applicable. Examples of suitable proteins and peptides range small proteins (e.g. small ubiquitin-related modifier) to antibodies (e.g. Rituximab).

An Acylation tag is an amino acid sequence comprising or consisting of a lysine residue and three or more histidine residues.

Acylating reagent refers to a reagent which facilitates site-selective acylation of the ε-amine of the lysine residue of the acylation tag of a target protein or peptide. It is a phenyl ester derivative having the formula (I) or (II):

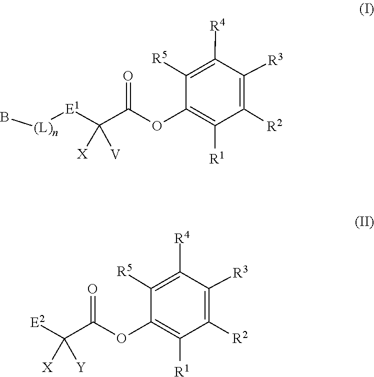

wherein E¹ and E² are an electron-withdrawing group or an alkylidene group; wherein E¹ is an attachment point of a biointeractive agent or an analytical agent; wherein E² is a reactive group that facilitates attachment to a biointeractive agent or to an analytical agent; wherein B is the biointeractive agent or analytical agent, wherein L is a linker, and n is 0 or 1; wherein the electron withdrawing group E¹ is selected from —C(O)O—, —OC(O)—, —NHC(O)—, —C(O)NH—, —O—, —NH—, —S—, —C(O)—, —OC(O)NH—, —NHC(O)—O—, —NHC(O)—NH—, —NHC(S)—NH—, —NHS(O)₂—, —S(O)₂NH—, ═NH—O—, ═NH—NH—, ═NH—N(alkyl)-, triazole, and the electron withdrawing group E² is selected from azide, alkyne, ring-strained alkyne, ring-strained alkene, tetrazine, nitrone, hydroxylamine, hydrazine, carbonyl, and phosphine; wherein X and Y are selected from hydrogen, an alkyl (e.g. methyl), a substituted alkyl, and an aryl group; and wherein R¹, R², R³, R⁴, and R⁵ are selected from hydrogen, an alkyl (e.g. methyl), an alkoxy (e.g. methoxy), and a halogen (e.g. Cl or F).

Biointeractive agent refers to an organic moiety that invokes a biological response when introduced into a living cell or tissue; examples of biointeractive agents include small molecules and macromolecules, such as toxins or therapeutic molecules.

Analytical agent refers to an organic moiety that can be detected by instrumental methods for qualitative or quantitative characterization of the material to which the analytical agent is bound; examples of analytical agents include labels such as fluorophores or radio labels.

An acylated protein or peptide of the present invention refers to target protein or peptides which have become modified (acylated) at the ε-amine of the acylation tag lysine residue due to contact with an acylating reagent.

Conjugated protein or peptide refers in the present invention to the biointeractive or analytical agent being attached (conjugated) to the modified target protein or peptide.

### DETAILED DESCRIPTION OF THE INVENTION

The present invention provides a protein or peptide comprising one of a variety of alternative Lys-His tags, all of which are able to undergo site-selective Lys acylation, and may further have the ability to bind to immobilized metal ions. The Lys-His tags in the proteins or peptides of the present invention can be located in regions distinct from the N-terminus of a protein, such as in loops or at the C-terminus. The Lys-His tags can be used to efficiently couple various functional groups, such as biotin and fluorophores, to a variety of proteins or peptides. The site-specific Lys acylation can further be can further be applied to antibodies, which owing to their size and hence large number of lysine residues represent a more challenging class of proteins. Lastly, the present invention provides a means for the selective modification of a protein or peptide of interest in a mixture of other proteins and/or peptides, providing proof of concept for its applicability in more complex biological systems.

It is anticipated that the present invention will be of great value to the fields of biotechnology, biopharmaceuticals and chemical biology. Further, a site-selective protein modification method is provided, that, because of its operational simplicity, robustness, mildness and anticipated scalability, has the potential to become the method of choice for a wide array of applications in both research and industry.

The method relies on the introduction of an acylation tag in the form of an amino acid sequence into a recombinant protein or peptide, said amino acid sequence comprising a Lys residue and 3 or more His residues. The ε-amine of the Lys residue reacts efficiently and selectively with an acylating reagent—preferably a 4-methoxy phenyl ester—as the His residues assist in deprotonation during the acylation reaction, hence the reaction is autocatalytic. As illustrated in the examples herein, it has been demonstrated that the acylation reaction works, resulting in the introduction of an azide moiety in the protein. Further, the direct introduction of a biotin group was demonstrated. The degree of functionalization can be determined by mass spectrometry.

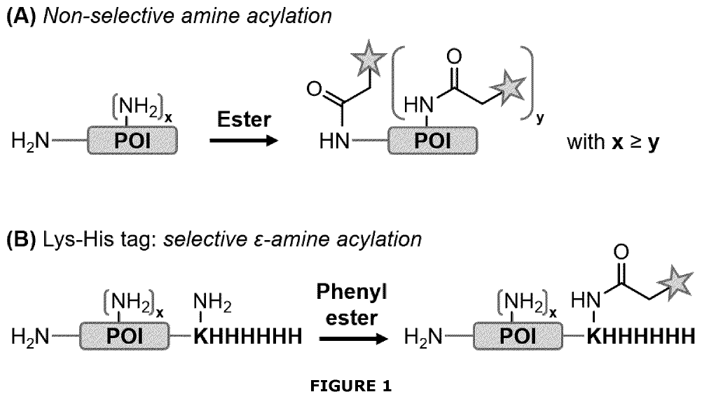

Illustration of the present invention compared to prior art is shown in FIG. 1

It was further shown that the introduced azide was available for attachment of a second molecule—e.g. conjugation with an alkyne-functionalized cyanine dye.

### I. Method for Site-Selective Modification of Proteins or Peptides

The present invention provides a method for site-selective modification of a target protein or peptide comprising the steps of:
a. providing a target protein or peptide comprising an acylation tag,
b. contacting the target protein or peptide from step (a) with an acylating agent to form a modified protein,
wherein said acylation tag comprises a lysine residue and at least three histidine residues, and wherein the target protein or peptide is modified at the ε-amine of the acylation tag lysine residue.

Site-selective modification is facilitated by the ε-amine of the acylation tag Lys residue reacting efficiently and selectively with the acylating reagent, as the presence of the acylation tag His residues allows for efficient deprotonation during the acylation reaction.

### I.i Acylation Tag

For site-selective modification of a target protein or peptide, according to the present invention, the target protein or peptide must comprise an acylation tag as specified further herein. The acylation tag must comprise a lysine residue and at least three histidine residues.

In one embodiment, the acylation tag in its simplest form comprises or consists of one lysine residue and three or more histidine residues, wherein the histidine residues are located adjacent to the lysine residue. In other words, in one embodiment, the acylation tag comprises or consists of an amino acid sequence selected from:

i.
(His)ₐ-Lys
and
ii.
Lys-(His)ₐ,

wherein a≥3.

In one embodiment, the lysine residue of the acylation tag is spaced from histidine residues by 1, 2, 3 or more other amino acid residues. In other words, in one embodiment, the acylation tag comprises or consists of an amino acid sequence selected from:

iii.
(His)ₐ-(X¹)_{b}-Lys,
and
iv.
Lys-(X¹)_{b}-(His)ₐ,

wherein a≥3 and b≥1, preferably between 1-3, and
wherein X¹ is one or more identical or different amino acids but not lysine.

In another embodiment, the acylation tag may comprise histidine residues on both sides of the lysine residue. In other words, in one embodiment, the acylation tag comprises or consists of an amino acid sequence selected from:

v.
(His)ₐ-(X¹)_{b}-Lys-(X²)_{c}-(His)_{d},
and
vi.
(His)_{d}-(X²)_{c}-Lys-(X¹)_{b}-(His)ₐ

wherein a≥3, b 0, preferably between 0-3, c 0, preferably between 0-3, and d 1, and
wherein X¹ and X² each are one or more identical or different amino acids but not lysine.

Having a greater number of His residues may facilitate improved acylation, and further—if the His residues are placed adjacent to one another—it may facilitate a means for purification of the tagged protein by metal affinity chromatography.

In the abovementioned embodiments, b and c refer to the number of amino acids separating Lys and His. Preferably, the Lys and His residues of the tag are in close proximity, hence, preferably b and c are 0, 1, 2 or 3—but in some cases the distance may be bigger. The amino acid(s) separating Lys and His are in the above denoted X¹ and X². X¹ and X² may each be one or more identical or different amino acids selected from any natural or non-natural amino acids except lysine. In one embodiment, X¹ and X² may be one or more amino acid(s) selected from alanine, arginine, asparagine, aspartate, cysteine, glutamate, glutamine, glycine, histidine, isoleucine, leucine, methionine, phenylalanine, proline, serine, threonine, tryptophan, tyrosine, valine, selenocysteine, and pyrrolysine.

In yet another embodiment, the at least three histidine residues of the acylation tag varieties mentioned above are not directly adjacent to one another. In other words, one or more of the histidine residues of the acylation tag may be spaced from the other histidine residues by one or more other amino acids. Preferably, the spacing between histidine residues of the acylation tag is not more than 1, 2, or 3 amino acid residues. In one embodiment, the total number of amino acids in the acylation tag is 25 or less, such as 24, 23, 22, 21, 20, 19, 18, 17, 16, 15, 14, 13, 12, or 11 amino acids; or even only 10, 9, 8, 7, 6, 5 or 4 amino acids in length.

In one embodiment, the acylation tag of the present invention is positioned at the C-terminus of the target peptide or protein. The sequence of the target peptide or protein is therefore as such not disrupted, but the tag is simply added to the C-terminus of the peptide or protein. An example of a C-terminally tagged protein is provided in Example 1.

In another embodiment, the acylation tag of the present invention is positioned internally within the target peptide or protein. The amino acid sequence of the target peptide or protein is thereby disrupted by the tag, such that a part of the target sequence is on one side of the acylation tag and the remaining part of the target sequence is on the other side of the tag. Preferably, the three-dimensional structure of the target peptide or protein is assessed prior to determining the internal positioning of the acylation tag. Thereby—in a preferred embodiment—with structural knowledge of the folded peptide or protein, the tag is located internally at a position which upon folding of the peptide or protein will be at the surface of the folded peptide or protein, thereby exposing the tag to the surrounding environment and making it easily accessible for interaction with the acylation reagent. Also, in a preferred embodiment, the position of the tag does not modify the overall structure of the peptide or protein as such, thereby ensuring the peptide or protein retains the functional properties of the untagged native peptide or protein. An example of an internally tagged protein is provided in Example 2.

### I.ii Providing a Target Peptide or Protein Comprising the Acylation Tag

The provided Lys-His tags is preferably inserted into any part of a protein structure, which does not constitute well-defined secondary structure (α-helix and β-strand/-sheet) or is part a of chain segment essential for protein folding or function. The Lys-His tags are therefore preferably engineered into N-terminal, mid-chain, and C-terminal regions, which constitute loops and other dynamic segments as characterized by the solved structure of the protein of interest, or in linker regions connecting different protein domains and/or proteins in fusion constructs. In addition, the Lys-His tags can be part of any other non-native sequence/structure that is inserted into a protein of interest (e.g. other protein tags, inteins, etc.).

The preferred location of the acylation tag may be decided based on structural knowledge and analysis of the target peptide or protein, as performed by a person skilled in the art. In cases where no 3D structure is available for the protein of interest, it can be predicted by freely available computational solutions, including automated protein homology modeling programs and automated online services such as CPHmodels (Technical University of Denmark)⁹, Phyre2 (Imperial College London)¹⁰, SWISS-MODEL (Swiss Institute of Bioinformatics)¹¹, ROSETTA¹², etc.

A target peptide or protein comprising an acylation tag at a desired position may be provided by standard lab procedures of chemical synthesis or recombinant expression or a combination of both methods.

In one embodiment, the target peptide or protein comprising an acylation tag is chemically synthesized as routinely performed by a person skilled in the art.

In another embodiment, the target peptide of protein comprising an acylation tag is recombinantly expressed in a suitable host as routinely performed by a person skilled in the art. For recombinant expression, an expression vector comprising a nucleic acid sequence encoding the amino acid sequence of the target peptide or protein comprising the acylation tag will typically be prepared by conventional methods. The host for expressing the recombinant protein or peptide may be selected from a prokaryotic host or eukaryotic host. In one embodiment, the target peptide or protein comprising the acylation tag is expressed in a prokaryotic host, such as Escherichia coli, Bacillus, Staphylococcus and other relevant prokaryotes. In one embodiment, the target peptide or protein comprising the acylation tag is expressed in yeast or fungi, such as Pichia, Saccharomyces, Aspergillus, Trichoderma, and Schizophyllum. In one embodiment, the target peptide or protein comprising the acylation tag is expressed in mammalian cells, such as CHO cell lines, COS cell lines, NSO cells, Syrian Hamster Ovary cell lines, HeLa cells, and human embryonic kidney cell lines.

After synthesis or expression, the peptide or protein may be purified and isolated by conventional purification techniques, such as solvent extraction, column chromatography (e.g. size exclusion chromatography, metal-affinity chromatography), and crystallization, or other purification techniques as recognized by a person skilled in the art.

### I.iii Acylating Reagent

Several acylating reagents are known in the art and are used for modifying and functionalizing proteins. However, not all acylating reagents facilitate site-specific acylation. For example, as illustrated in Example 4, when using a commonly used acylating reagent NHS (N-hydroxysuccinimide) ester the modification is prone to random/non-specific labeling.

Acylating reagents of the present invention provide selective and efficient acylation of a Lys side chain in an autocatalytic reaction as described. The acylating reagents of the present invention are less reactive than common reagents for N-acylation of non-activated amines, such as N-hydroxysuccinimide esters of acids and amino acids. Examples of preferred acylating reagents of the present invention include phenyl esters carrying 0, 1, 2, 3, 4 or 5 substituents selected from one or more of alkyl, alkoxy, and/or halogen (e.g. Cl and F).

In one embodiment, the acylating reagent of the present invention is a phenyl ester derivative. In one embodiment, the acylating reagent is a phenyl ester derivative having the formula (I) or (II):

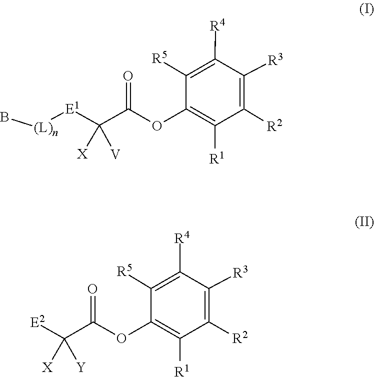

wherein E¹ and E² are an electron-withdrawing group or an alkylidene group; wherein E¹ is an attachment point of a biointeractive agent or an analytical agent; wherein E² is a reactive group that facilitates attachment to a biointeractive agent or to an analytical agent; wherein B is the biointeractive agent or analytical agent, wherein L is a linker, and n is 0 or 1; wherein the electron withdrawing group E¹ is selected from —C(O)O—, —OC(O)—, —NHC(O)—, —C(O)NH—, —O—, —NH—, —S—, —C(O)—, —OC(O)NH—, —NHC(O)—O—, —NHC(O)—NH—, —NHC(S)—NH—, —NHS(O)₂—, —S(O)₂NH—, ═NH—O—, ═NH—NH—, ═NH—N(alkyl)-, triazole, and the electron withdrawing group E² is selected from azide, alkyne, ring-strained alkyne, ring-strained alkene, tetrazine, nitrone, hydroxylamine, hydrazine, carbonyl, and phosphine; wherein X and Y are selected from hydrogen, an alkyl (e.g. methyl), a substituted alkyl, and an aryl group; and wherein R¹, R², R³, R⁴, and R⁵ are selected from hydrogen, an alkyl (e.g. methyl), an alkoxy (e.g. methoxy), and a halogen (e.g. Cl or F).

In one preferred embodiment, the acylating reagent is a phenyl ester derivative having the formula (I) or (II), wherein E1 and E2 are an electron-withdrawing group or an alkylidene group; wherein E1 is an attachment point of a biointeractive agent or an analytical agent; wherein E2 is a reactive group that facilitates attachment to a biointeractive agent or to an analytical agent; wherein B is the biointeractive agent or analytical agent, wherein L is a linker, and n is 0 or 1; wherein the electron withdrawing group E1 is selected from —C(O)O—, —OC(O)—, —NHC(O)—, —C(O)NH—, —O—, —NH—, —S—, —C(O)—, —OC(O)NH—, —NHC(O)—O—, —NHC(O)—NH—, —NHC(S)—NH—, —NHS(O)₂—, —S(O)₂NH—, ═NH—O—, ═NH—NH—, ═NH—N(alkyl)-, triazole, and the electron withdrawing group E2 is selected from azide, a C₂-C₈ alkyne, a ring-strained alkyne with a ring-size of C₇-C₉ (e.g. DBCO, DIFO, BCN), a ring-strained alkene with a ring-size of C3-C9 (e.g. trans-cyclooctene, cyclopropene), tetrazine, nitrone, hydroxylamine, hydrazine, carbonyl, and phosphine; wherein X and Y are selected from hydrogen, a short-chain alkyl (C₁-C₄, e.g. methyl), and an aryl group (C6-C10, e.g. phenyl); wherein R4 and R5 are hydrogen; wherein R1, R2, and R3 are selected from hydrogen, a short-chain alkyl (C₁-C₄, e.g. methyl), an alkoxy with a short alkyl chain (C1-C4, e.g. methoxy), and halogen (e.g. Cl or F), and wherein at least one of R1 and R3 is an electron-donating moiety (i.e. a moiety that donates electron-density into the aromatic ring, for example, methoxy or methyl).

In one embodiment, E² is a reactive group that facilitates attachment to a biointeractive agent or to an analytical agent. In one embodiment, this reactive group that facilitates attachment to a biointeractive agent or to an analytical agent is selected from azide, alkyne, ring-strained alkyne (e.g. DBCO, DIFO, BCN), ring-strained alkene (e.g. trans-cyclooctene, cyclopropene), tetrazine, nitrone, hydroxylamine, hydrazine, carbonyl, and phosphine. In a preferred embodiment, the reactive group that facilitates attachment to a biointeractive/analytical agent is azide.

In one embodiment, E¹ is the attachment point of a biointeractive or analytical agent, optionally via a linker. In one embodiment, E¹ is a chemical group selected from —C(O)O—, —OC(O)—, —NHC(O)—, —C(O)NH—, —O—, —NH—, —S—, —C(O)—, —OC(O)NH—, —NHC(O)—O—, —NHC(O)—NH—, —NHC(S)—NH—, —NHS(O)₂—, —S(O)₂NH—, ═NH—O—, ═NH—NH—, ═NH—N(alkyl)-, triazole, and any combination thereof.

In one embodiment, B is a biointeractive or analytical agent selected from biotin, a fluorophore (such as Alexa Fluor dyes, Fluorescein, Cyanine dyes, ATTO dyes), a toxin, Mycotoxins (aflatoxin), Paralytic shellfish toxins (saxitoxin), Auristatins), a chelator (such as Dodecane tetraacetic acid (DOTA), Nitrilotriacetic acid (NTA), Bipyridine), a half-life extending moiety (such as Polyethylene glycol (PEG), XTEN, Elastin-like polypeptides, Proline/alanine-rich sequence (PAS) polypeptides, Fatty acid, Small-molecule albumin binders, Cholesterol-like half-life extenders), an imaging reagent (such as Fluorophores (see above), Radioactive label, Phosphorescent label, Quantum dot), a crosslinking moiety (such as Benzophenone, Diazirine, Halogen (F, Cl, Br, and I), Phenylazide), a peptide, a protein, an oligonucleotide, a lipid, a mono- or polysaccharide, a synthetic polymer, and a viral particle.

The linker (L)—if present—separates the target protein or peptide and the biointeractive/analytical agent or any moiety comprising a reactive group which facilitates covalent attachment to the biointeractive/analytical agent. Its chemical structure is not critical, since it serves primarily as a spacer. In one embodiment, the linker comprises or consists of a chemical group selected from an alkyl group, an alkenyl group, a cycloalkyl group, an aryl group, a heteroaryl group, a heterocyclic group, a polyethylene glycol, a natural amino acid, an unnatural amino acid, and any combination thereof.

In the present invention, the reactivity of the acylating reagent can be adjusted by modification on either sides of the ester functional group, thereby adjusting the electro-withdrawing properties, as recognized by a person skilled in the art. The reactivity of the acylating reagent can be adjusted by varying the substituents on a phenyl ester (R¹, R², R³, R⁴, and R⁵) or by modifying the E¹ or E² on the carbonyl side of the ester.

4-Methoxyphenyl 2-azidoacetate is used as an illustrative example. 4-Methoxyphenyl 2-azidoacetate is a phenyl ester derivative according to formula (II), wherein E² is N₃, R¹, R², R⁴, and R⁵ are hydrogen, and R³ is methoxy—as shown in formula (III):

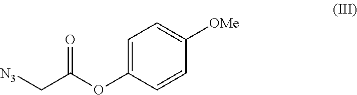 [SMILES: COc1ccc(OC(=O)CN=[N+]=[N-])cc1]

In formula (III), the azide group on the carbonyl side (E² of formula II) of the ester in 4-methoxyphenyl 2-azidoacetate is an electron-withdrawing group. If the E² group of formula (II) is less electron-withdrawing than an azide group, the R¹, R², R³, R⁴, and R⁵ groups of formula (II) may have to be more electron-withdrawing to compensate and achieve a suitable reactivity of the acylating reagent. An example of E² being less electron-withdrawing than an azide group may be if E² is alkyne, ring-strained alkyne, ring-strained alkene, or tetrazine; in such embodiment, one or more of R¹, R², R³, R⁴, and R⁵ of formula (II) may have to be more electron-withdrawing than a single methoxy (R³) shown in formula III to compensate and achieve a suitable reactivity of the acylating reagent.

In one embodiment, the acylating reagent of the present invention is a phenyl ester derivative having a structure according to formula (II), wherein E² is N₃—; X and Y are hydrogen; while the substituents R¹, R², R³, R⁴, and R⁵ on the phenyl ring are selected from one of the following combinations: (i) R¹, R², R⁴, and R⁵ are hydrogen, and R³ is methoxy, (ii) R¹, R⁴ and, R⁵ are hydrogen, R² is chloride or fluoride, and R³ is methoxy, (iii) R¹ is chloride or fluoride, R², R⁴, and R⁵ are hydrogen, and R³ is methoxy, and (iv) R¹, R⁴, and R⁵ is hydrogen, and R² and R³ are methoxy.

In one embodiment, the acylating reagent of the present invention is a phenyl ester derivative having a structure according to formula (I), wherein B is biotin; L is oligoethylene glycol; n=1; E¹ is —C(O)—NH— or —O—; X and Y are hydrogen; while the substituents R¹, R², R³, R⁴, and R⁵ on the phenyl ring are selected from one of the following combinations: (i) R¹, R², R⁴, and R⁵ are hydrogen, and R³ is methoxy, (ii) R¹ is fluoride, R², R⁴, and R⁵ are hydrogen, and R³ is methoxy, and (iii) R¹ is fluoride, R² is chloride, R³ is methoxy, and R⁴ and R⁵ are hydrogen.

In one embodiment, the acylating reagent of the present invention is a phenyl ester derivative having a structure according to formula (I), wherein B is biotin; n=0; E¹ is —CH₂—; X and Y are hydrogen; while R¹, R⁴, and R⁵ is hydrogen, R² is cloride or fluoride, and R³ is methoxy.

In one embodiment, the acylating reagent of the present invention is a phenyl ester derivative having a structure according to formula (I), wherein B is biotin; n=0; E¹ is —CH₂—; X and Y are hydrogen; while R¹, R², R³, R⁴, and R⁵ are hydrogen. In one preferred embodiment, the acylating reagent of the present invention is a 4-methoxy phenyl ester derivative having the formula (IV) or (V):

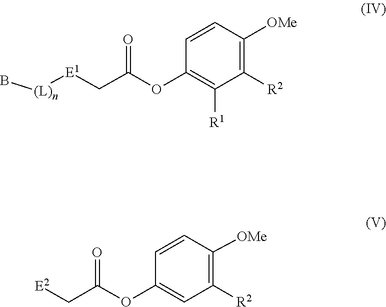

wherein B is a biointeractive agent or an analytical agent; L is a linker; n is 0 or 1; E¹ is selected from —C(O)O—, —OC(O)—, —NHC(O)—, —C(O)NH—, —O—, —NH—, —S—, —C(O)—, —OC(O)NH—, —NHC(O)—O—, —NHC(O)—NH—, —NHC(S)—NH—, —NHS(O)₂—, —S(O)₂NH—, ═NH—O—, ═NH—NH—, ═NH—N(alkyl)-, and triazole; E² is selected from azide, alkyne, ring-strained alkyne, ring-strained alkene, tetrazine, nitrone, hydroxylamine, hydrazine, carbonyl and phosphine; and R² is selected from a hydrogen, an alkyl (e.g. methyl), an alkoxy (e.g. methoxy), and a halogen (e.g. Cl or F).

In a preferred embodiment, the acylating reagent of the present invention is a phenyl ester derivative having a structure according to formula (IV) or (V), wherein B is a biointeractive agent or an analytical agent; L is a linker; n is 0 or 1; E¹ is selected from —C(O)O—, —OC(O)—, —NHC(O)—, —C(O)NH—, —O—, —NH—, —S—, —C(O)—, —OC(O)NH—, —NHC(O)—O—, —NHC(O)—NH—, —NHC(S)—NH—, —NHS(O)₂—, —S(O)₂NH—, ═NH—O—, ═NH—NH—, ═NH—N(alkyl)-, and triazole; E2 is selected from azide, a C₂-C₈ alkyne, a ring-strained alkyne with a ring-size of C₇-C₉ (e.g. DBCO, DIFO, BCN), a ring-strained alkene with a ring-size of C₃-C₉ (e.g. trans-cyclooctene, cyclopropene), tetrazine, nitrone, hydroxylamine, hydrazine, carbonyl, and phosphine; and R² is selected from hydrogen, a short-chain alkyl (C1-C4, e.g. methyl), an alkoxy with a short alkyl chain (C1-C4, e.g. methoxy), and halogen (e.g. Cl or F).

In a most preferred embodiment, the acylating reagent of the present invention is 4-methoxyphenyl 2-azidoacetate having formula (III):

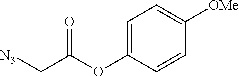 [SMILES: COc1ccc(OC(=O)CN=[N+]=[N-])cc1]

As mentioned previously, the reactivity of the acylating reagent can be adjusted by modification on either sides of the ester functional group. In a preferred embodiment, the electro-withdrawing property of the acylating reagent of the present invention resembles that of 4-methoxyphenyl 2-azidoacetate (formula (III)). A person skilled in the art will recognize that the electron-withdrawing properties of the acylating reagent of the present invention can be adjusted by varying the substituents on the phenyl ester (R¹, R², R³, R⁴, and R⁵) and/or by modifying the E¹ or E² on the carbonyl side of the ester of formula (I) or (II). Such person skilled in the art will likewise know how to balance the reactivity (electron-withdrawing property) of the different R- and E-groups of the acylating reagent to obtain a reactivity resembling the reactivity of 4-methoxyphenyl 2-azidoacetate.

### I.iv Providing Acylating Reagent

The acylating reagents are phenyl esters. They are chemically synthesized by ester formation from (a) reagents carrying the desired biointeractive agent or analytical agent or reactive moiety and a carboxylic acids and (b) phenols. Alternatively, the phenyl ester is synthesized in a first step and is subsequently modified to introduce the desired biointeractive agent or analytical agent or reactive moiety. Example 9 discloses the synthesis of selected acylating reagents of the present invention.

### I.v Selective Acylation of the ε-Amine of the Acylation Tag Lys Residue

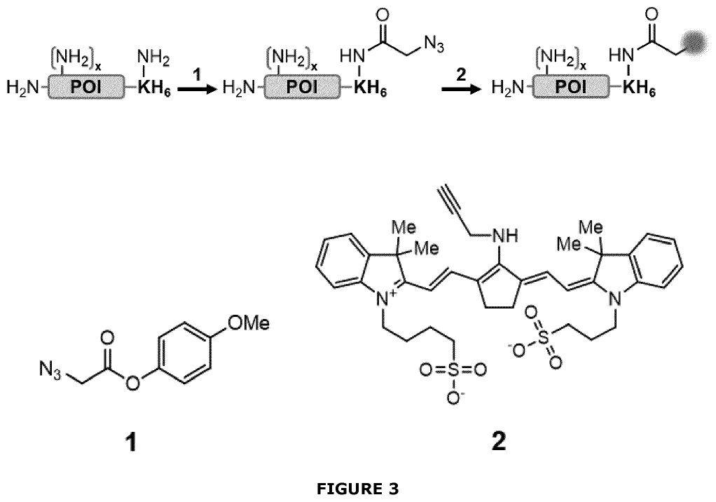

Acylation of the ε-amine of the acylation tag lysine residue may be presented as illustrated in FIG. 3, reaction 1. A target protein is provided comprising a (Lys)(His)₆ tag at the C-terminus, and said protein is contacted with reagent 1 (4-methoxyphenyl 2-azidoacetate), yielding a modified protein comprising a reactive group (—N₃) which facilitates attachment to a biointeractive/analytic agent.

The acylation reaction is carried out in aqueous media.

The acylation reaction may preferably be carried out at a temperature at which the target peptide or protein is stable. Further, the acylation reaction is preferably carried out at a relatively low temperatures due to the increased stability of the acylating reagent, where e.g. azido phenyl esters have longer half-lives at lower temperatures, leading to higher conversion. In one embodiment, the acylation reaction is carried out at a temperature between 1-50° C., such as between 2-37° C., 2-20° C., or preferably 2-10° C. In one embodiment, the acylation reaction is carried out at a temperature below 50° C., such as below 45, 40, 35, 30, 25, 20 or 15° C., such as preferably below 10° C. In one preferred embodiment, the acylation reaction is carried out at a temperature at 4° C.

The acylation reaction may be preferably carried out at a pH at which the target peptide or protein is stable. Further, the acylation reaction is preferably carried out at a pH range that ensures stability and functionality of the acylating reagent, as e.g. high pH will render the ester prone to hydrolysis, while a low pH the lysine of the acylation tag will become preferentially protonated and non-functional. It is in other words important to use a high enough pH such that the lysine in the acylation tag can get readily deprotonated (with help from the histidine residues), but not so high that other lysine residues are deprotonated (to prevent off-target acylation). The acylation reaction is preferably carried out in an aqueous solution buffered to between pH 6-9, such as between pH 6.5-8.5, such as between 7-8.5, such as between 7.5-8, preferably between pH 7-8. The buffered solution should not contain primary amines, but could be selected from phosphate buffers, HEPES, MOPS and PIPES, as recognized by a person skilled in the art.

In a further embodiment, the acylation reaction may be performed in the presence of EDTA to capture any free divalent metal ions. In one embodiment, EDTA is added to the acylation reaction to a final concentration of between 0.01-10 mM, such as 0.05-5 mM, such as 0.1-1 mM EDTA.

### I.vi Site-Selective Conjugation

The resulting modified protein or peptide—after the acylation reaction—now comprises a reactive group which in a following step can react with available reactive functionalities on an analytical or biointeractive agent to form a covalent bond—thereby facilitating site-specific conjugation, such as illustrated in FIG. 3, reaction 2, where the modified protein is contacted with an analytic agent (reagent 2: alkyne cyanine dye), yielding a protein conjugate easily detectable.

In one embodiment, the reactive group on the modified protein or peptide for taking part in conjugation is selected from azide, alkyne, ring-strained alkyne, ring-strained alkene, tetrazine, nitrone, hydroxylamine, hydrazine, carbonyl, and phosphine.

In one embodiment, the reactive functionalities on the analytical or biointeractive agent is selected from azide, alkyne, ring-strained alkyne, ring-strained alkene, tetrazine, nitrone, nitrone, hydroxylamine, hydrazine, carbonyl, and phosphine.

Reactive group and functional group pairs include: (1) Azide to undergo a Huisgen cycloaddition with an alkyne and more particularly a cyclooctyne reactive group (more commonly known as click chemistry), or to undergo a Staudinger ligation with a phosphine; (2) Carbonyl group to react with a reactive group selected from hydroxylamine or hydrazine to form oxime or hydrazine respectively; (3) Ring-strained alkene or ring-strained alkyne to react with a tetrazine reactive group in an aza [4+2] addition.

In a preferred embodiment, the reactive group of the modified target protein or peptide is terminal or ring-strained alkyne, for conjugating said target protein or peptide to the biointeractive or analytical agent comprising an azide group.

In one embodiment, the biointeractive or analytical agent conjugated to the modified target protein or peptide is selected from biotin, fluorophore, toxin, chelator, a half-life extending moiety, an imaging reagent, a crosslinking moiety, a peptide, a protein, an oligonucleotide, a lipid, a mono- or polysaccharide, a synthetic polymer and a viral particle.

### II. Products of the Present Invention

### II.i Acylated Proteins or Peptides Obtainable by the Present Invention

In one embodiment, the present invention provides an acylated protein or peptide obtained by the method of the invention, wherein said acylated protein or peptide comprises an acylation tag as defined herein, comprising a lysine residue and at least three histidine residues, wherein said acylation tag is located internally or at the C-terminus of the protein, and wherein the acylation is site-specific at the lysine residue of the acylation tag. The acylated protein comprises a reactive group as defined herein, specifically at the lysine residue of the acylation tag.

In one embodiment, the present invention provides an acylated protein or peptide comprising an acylation tag, wherein the acylation tag comprises or consists of an amino acid sequence tag selected from:

i.
(His)ₐ-(X¹)_{b}-Lys,
ii.
Lys-(X¹)_{b}-(His)ₐ,
iii.
(His)ₐ-(X¹)_{b}-Lys-(X²)c-(His)ₐ
and
iv.
(His)_{d}-(X²)_{c}-Lys-(X¹)_{b}-(His)ₐ

wherein a≥3, b=0-3, c=0-3, and d 1, and wherein X¹ and X² each are one or more identical or different amino acids but not lysine, and wherein the ε-amine of Lys in said sequence tag is acylated.

### II.ii Protein or Peptide Conjugates Obtainable by the Present Invention

In one embodiment, the present invention provides protein or peptide conjugates obtained by the method of the invention, wherein said protein or peptide conjugates comprise a target protein or peptide conjugated to a biointeractive or analytical agent, wherein the target protein or peptide comprises an acylation tag as defined herein, comprising a lysine residue and at least three histidine residues, wherein said acylation tag is located internally or at the C-terminus of the target protein, and wherein the biointeractive or analytical agent is site-specifically conjugated to the target protein or peptide via the lysine residue of the acylation tag. The biointeractive or analytical agent is covalently attached to the target protein or peptide by interaction between the reactive group of the acylated target protein or peptide and the functional group of the biointeractive/analytical agent, as described herein.

In one embodiment, the present invention provides a conjugated protein or peptide comprising an acylation tag, wherein the acylation tag comprises or consists of an amino acid sequence selected from:

i.
(His)ₐ-(X¹)_{b}-Lys,
ii.
Lys-(X¹)_{b}-(His)ₐ,
iii.
(His)ₐ-(X¹)_{b}-Lys-(X²)c-(His)ₐ
and
iv.
(His)_{d}-(X²)_{c}-Lys-(X¹)_{b}-(His)ₐ

wherein a≥3, b=0-3, c=0-3, and d 1, wherein X¹ and X² each are one or more identical or different amino acids but not lysine, and wherein the Lys in said sequence tag is conjugated to a biointeractive agent or an analytical agent.

### III. Compositions of the Present Invention

In one aspect, the present invention further provides an aqueous composition comprising an acylated protein or peptide and/or a conjugated protein or peptide as described herein.

### IV. A Kit for Site-Selective Modification of a Target Protein or Peptide

In one aspect, the invention provides a kit for modifying a target protein or peptide, wherein said kit comprises
a. a target protein or peptide, or a nucleic acid sequence encoding same, wherein said target protein or peptide comprises an acylation tag, as described herein, and
b. an acylating reagent, as described herein.

In one embodiment, the kit of the invention comprises
a. a target protein or peptide, or a nucleic acid sequence encoding same, wherein said target comprises an acylation tag, wherein the acylation tag comprises or consists of an amino acid sequence selected from:

i.
(His)ₐ-(X¹)_{b}-Lys,
ii.
Lys-(X¹)_{b}-(His)ₐ,
iii.
(His)ₐ-(X¹)_{b}-Lys-(X²)c-(His)ₐ
and
iv.
(His)_{d}-(X²)_{c}-Lys-(X¹)_{b}-(His)ₐ

wherein a≥3, b=0-3, c=0-3, and d 1, and wherein X¹ and X² each are one or more identical or different amino acids but not lysine.
b. an acylating reagent having the formula (I) or (II)

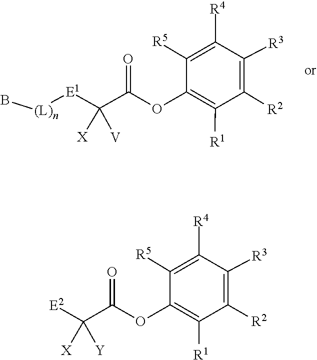

wherein E¹ and E² are an electron-withdrawing group or an alkylidene group; wherein E¹ is an attachment point of a biointeractive agent or an analytical agent; wherein E² is a reactive group that facilitates attachment to a biointeractive agent or to an analytical agent; wherein B is the biointeractive agent or analytical agent, wherein L is a linker, and n is 0 or 1; wherein the electron withdrawing group E¹ is selected from —C(O)O—, —OC(O)—, —NHC(O)—, —C(O)NH—, —O—, —NH—, —S—, —C(O)—, —OC(O)NH—, —NHC(O)—O—, —NHC(O)—NH—, —NHC(S)—NH—, —NHS(O)₂—, —S(O)₂NH—, ═NH—O—, ═NH—NH—, ═NH—N(alkyl)-, triazole, and the electron withdrawing group E² is selected from azide, alkyne, ring-strained alkyne, ring-strained alkene, tetrazine, nitrone, hydroxylamine, hydrazine, carbonyl, and phosphine; wherein X and Y are selected from hydrogen, an alkyl (e.g. methyl), a substituted alkyl, and an aryl group; and wherein R¹, R², R³, R⁴, and R⁵ are selected from hydrogen, an alkyl (e.g. methyl), an alkoxy (e.g. methoxy), and a halogen (e.g. Cl or F).

### V. Use of an Acylation Tag for Site-Selective Modification of a Target Protein or Peptide

In one aspect, the present invention concerns the use of an acylation tag as described herein for site-selective modification of a target protein or peptide.

In one embodiment, the present invention concerns the use of an acylation tag for site-selective modification of a target protein or peptide, wherein said acylation tag comprises an amino acid sequence located internally or at the C-terminus of said target protein or peptide, wherein said amino acid sequence of said acylation tag comprises a single lysine residue and at least three histidine residues; and wherein the ε-amine of the lysine residue of the acylation tag is capable of being acylated upon contact with an acylating reagent.

In one embodiment, the present invention concerns the use of an acylation tag for site-selective modification of a target protein or peptide, wherein said acylating reagent is a phenyl ester derivative of formula (I) or (II):

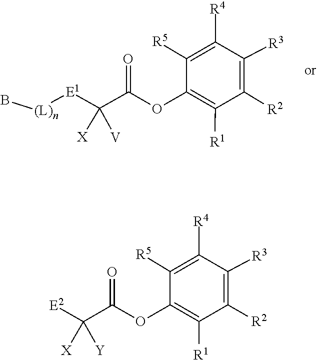

wherein E¹ and E² are an electron-withdrawing group or an alkylidene group; wherein E¹ is an attachment point of a biointeractive agent or an analytical agent; wherein E² is a reactive group that facilitates attachment to a biointeractive agent or to an analytical agent;
wherein B is the biointeractive agent or analytical agent, wherein L is a linker, and n is 0 or 1;
wherein the electron withdrawing group E¹ is selected from —C(O)O—, —OC(O)—, —NHC(O)—, —C(O)NH—, —O—, —NH—, —S—, —C(O)—, —OC(O)NH—, —NHC(O)—O—, —NHC(O)—NH—, —NHC(S)—NH—, —NHS(O)₂—, —S(O)₂NH—, ═NH—O—, ═NH—NH—, ═NH—N(alkyl)-, triazole,
wherein the electron withdrawing group E² is selected from azide, alkyne, ring-strained alkyne, ring-strained alkene, tetrazine, nitrone, hydroxylamine, hydrazine, carbonyl, and phosphine;
wherein X and Y are selected from hydrogen, an alkyl (e.g. methyl), a substituted alkyl, and an aryl group; and wherein R¹, R², R³, R⁴, and R⁵ are selected from hydrogen, an alkyl (e.g. methyl), an alkoxy (e.g. methoxy), and a halogen (e.g. Cl or F).

In a preferred embodiment, the present invention concerns the use of an acylation tag for site-selective modification of a target protein or peptide, wherein said acylating reagent is a 4-methoxyl phenyl ester derivative of formula (VI) or (V):

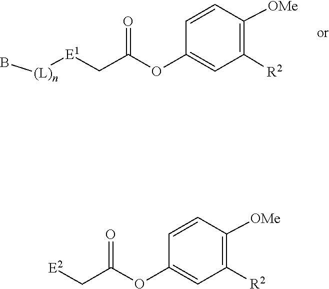

wherein B is a biointeractive agent or an analytical agent,
wherein L is a linker and n is 0 or 1,
wherein E¹ is selected from —C(O)O—, —OC(O)—, —NHC(O)—, —C(O)NH—, —O—, —NH—, —S—, —C(O)—, —OC(O)NH—, —NHC(O)—O—, —NHC(O)—NH—, —NHC(S)—NH—, —NHS(O)₂—, —S(O)₂NH—, ═NH—O—, ═NH—NH—, ═NH—N(alkyl)-, and triazole,
wherein E² is selected from azide, alkyne, ring-strained alkyne, ring-strained alkene, tetrazine, nitrone, hydroxylamine, hydrazine, carbonyl and phosphine, and
wherein R² is selected from hydrogen, an alkyl (e.g. methyl), an alkoxy (e.g. methoxy), and a halogen (e.g. Cl or F).

In a most preferred embodiment, the present invention concerns the use of an acylation tag for site-selective modification of a target protein or peptide, wherein said acylating reagent is 4-methoxyphenyl 2-azidoacetate having formula (II):

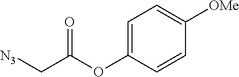 [SMILES: COc1ccc(OC(=O)CN=[N+]=[N-])cc1]

### VI. A Method of Detecting Products Produced by the Present Method

Methods for detecting acylated proteins or peptides produced by the method of the invention include mass spectrometry, in-gel fluorescence imaging, Western blot analysis, fluorescence microscopy, reverse-phase liquid chromatography (RP-HPLC), hydrophobic interaction chromatography (HIC), etc.; where the products may be identified and optionally quantified compared to known standards, as one ordinary skilled in the art would be familiar with. Example 1 comprises the outline of one method of detection and quantification of proteins.

### VII. Advantages and Commercial Application

### VII.i Medical and Diagnostic Applications of the Method of the Invention

In one embodiment, it may be desirable to modify proteins to alter the physicochemical properties of the protein/peptide, such as e.g. to increase (or to decrease) solubility to modify the bioavailability of a therapeutic protein. In another embodiment, it may be desirable to modify the clearance rate of a protein or peptide in the body by conjugating compounds to the protein/peptide that binds to plasma proteins, such as e.g. albumin, or which increase the size of the protein or peptide to prevent or delay discharge through the kidneys. Conjugation may also alter and in particular decrease the susceptibility of a protein/peptide to hydrolysis, such as e.g. in vivo proteolysis. It may also be desirable to modify the immunogenicity of a protein, e.g. by conjugating a protein so as to hide, mask or eclipse one or more immunogenic epitopes in the protein.

In another embodiment, the invention provides a method of improving pharmacological properties of a target protein or peptide. The improvement is with respect to the corresponding unmodified protein or peptide. Examples of such pharmacological properties include functional in vivo half-life, immunogenicity, renal filtration, protease protection and albumin binding or other plasma protein binding of any specific protein.

In one embodiment, the invention may provide antibody-drug conjugates, such as those designed as a targeted therapy for treating cancer.

In one embodiment, it may be desirable to conjugate a label to facilitate analysis of the protein/peptide. Examples of such labels include radioactive isotopes, phosphorescence markers, fluorescent markers such as the fluorophores already described, and enzymes.

In still another embodiment, a compound is conjugated to a protein to facilitate isolation of the protein. For example, a compound with a specific affinity to a particular column material may be conjugated to the protein.

### VII.i i Therapeutic Uses and Pharmaceutical Compositions

To the extent that the unmodified protein/peptide is a therapeutic protein or peptide, the invention also relates to the use of the modified protein or peptide in therapy, and in particular to pharmaceutical compositions comprising the modified proteins. The conjugate of the instant invention may be administered in any of a variety of ways, including subcutaneously, intramuscularly, intravenously, intraperitoneally, inhalationally, intranasally, orally etc.

### EXAMPLES

Table 1 provides an overview of the different acylating reagents and dyes used in the examples disclosed herein in support of the present invention.

TABLE 1

| # | Name | Structure |
|---|---|---|
| 1 | 4- Methoxyphenyl- 2-azidoacetate | 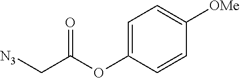 [SMILES: COc1ccc(OC(=O)CN=[N+]=[N-])cc1] |
| 2 | Alkyne cyanine dye 718, CAS no. 1188292-54-7 | 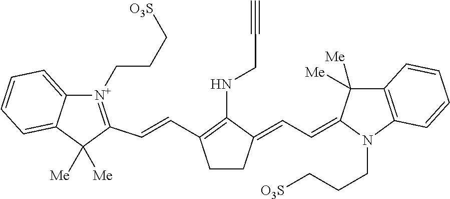 |
| 3 | 2-Azidoacetic acid N- hydroxy- succinimide ester | 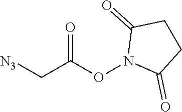 [SMILES: [N-]=[N+]=NCC(=O)ON1C(=O)CCC1=O] |
| 4a | 3,4- Dimethoxy- phenyl 2- azidoacetate | 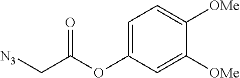 [SMILES: COc1ccc(OC(=O)CN=[N+]=[N-])cc1OC] |
| 4b | 3-Chloro-4- methoxy- phenyl 2- azidoacetate | 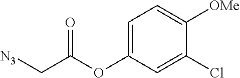 [SMILES: COc1ccc(OC(=O)CN=[N+]=[N-])cc1Cl] |
| 5 | D-Biotin N- hydroxy- succinimide ester | 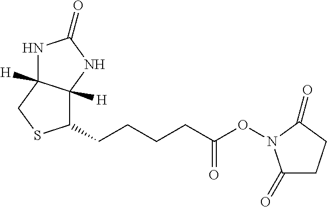 |
| 6a | 4- Methoxyphenyl 2-(2-(2-(D- biotinylamino) ethoxy)ethoxy) acetate | 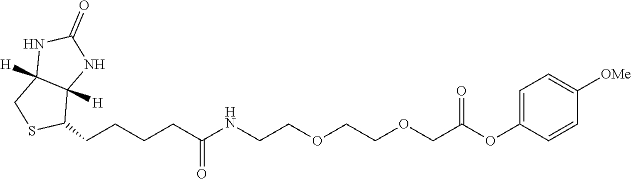 |
| 6b | 3-Chloro-4- methoxyphenyl 2-(2-(2-(D- biotinylamino) ethoxy) ethoxy)-acetate | 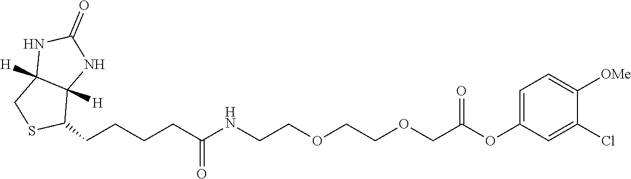 |

### Example 1: Site-Selective Acylation of Proteins with a C-Terminal Lys-His Tag

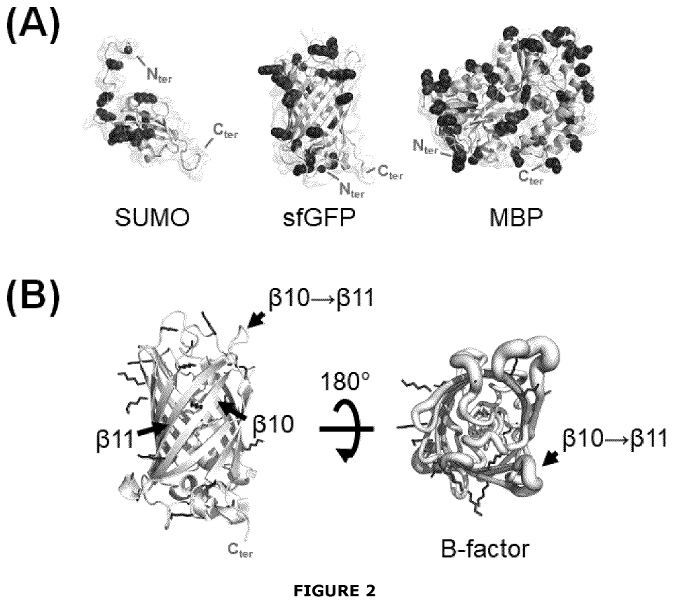

Two different small peptide tags: KHHHHHH (KH₆) (SEQ ID NO.: 62) and HHHKHHHHHH (H₃KH₆) (SEQ ID NO.: 63), were introduced at the C-terminus of three model proteins: small ubiquitin-related modifier (SUMO), super-folder green-fluorescent protein (sfGFP) and maltose-binding protein (MBP), all of which have multiple native, surface-exposed lysine residues (FIG. 2A).

FIG. 3 illustrates this by showing a protein of interest (POI) comprising a Lys-His tag (KH₆), first undergoing acylation by reaction with 4-methoxyphenyl 2-azidoacetate (1), then further conjugation with an alkyne-functionalized cyanine dye (2). 1.1 Preparing vector constructs comprising C-terminally tagged proteins Polymerase Chain Reaction: To amplify genes of interest with desired restriction digest sites Polymerase Chain Reaction (PCR) was employed. A PCR reaction was prepared by mixing in a PCR test tube 1 μL DNA template (1 ng/μL), 1 μL forward primer (10 μM), 1 μL reverse primer (10 μM), 1 μL dNTPs (10 mM), 4 μL Phusion® High-Fidelity Buffer (5×), 0.2 μL Phusion® High-Fidelity Polymerase (2,000 units/mL) and 11.8 μL ultrapure H₂O. The sequence of steps as detailed in Table 2 was performed in the PCR cycle. After completion of the PCR cycle, 4 μL Gel Loading Dye, Purple (6×) was added to the PCR test tube, and the mixture run on a 1% agarose gel for 30 min at 120 V. The band corresponding to the correct PCR product was excised from the gel and purified using a GeneJET Gel Extraction Kit and 30 μL elution. The purified PCR product was stored in a new test tube at −20° C. until further use.

TABLE 2
Polymerase Chain Reaction Sequence

| Step | Description | Temp. (° C.) | Time | |
|---|---|---|---|---|
| 1 | Initial denaturation | 98 | 1 | min |
| 2 | Denaturation | 98 | 30 | sec |
| 3 | Annealing | 50 | 20 | sec |
| 4 | Elongation | 72 | 210 | sec |

Repeat steps 2->3 for 36 cycles

| | | | | |
|---|---|---|---|---|
| 5 | Final extension | 72 | 10 | min |

| | | | |
|---|---|---|---|
| 6 | Hold | 10 | ∞ |

DNA restriction digest: A preparation double-stranded DNA with overhangs were generated by mixing in a test tube 20 μL DNA, 2.5 μL CutSmart Buffer (10×) buffer, 0.5 μL of each restriction enzyme (as specified), and 1.5 μL ultrapure H₂O. The restriction digest was incubated for 1 hr at 37° C. Then 5 μL Gel Loading Dye, Purple (6×) was added to the test tube, and the mixture run on a 1% agarose gel for 30 min at 120 V. The band corresponding to the desired digest product was excised from the gel and purified using a GeneJET Gel Extraction Kit. The purified digest product was stored in a new test tube at −20° C. until further use.

DNA dephosphorylation reaction: A preparation of double-stranded DNA with dephosphorylated ends was generated by mixing in a test tube 20 μL DNA, 2.5 μL rSAP buffer (10×), 1 μL shrimp alkaline phosphatase (1,000 units/mL), and 1.5 μL ultrapure H₂O. The dephosphorylation reaction was incubated for 30 min at at 37° C. Shrimp alkaline phosphatase was inactivated by incubating the test tube for 5 min at 65° C. The dephosphorylation reaction was then stored in the test tube at −20° C. until further use.

DNA ligation reaction: The final preparation of an expression vector was generated by ligation between the gene of interest and the designated vector. The ligation reaction was done by mixing in a test tube 0.020 pmol vector (linearized and dephosphorylated), 0.060 pmol DNA insert with complementary overhangs, 1.5 μL T4 DNA ligase buffer (10×), 1 μL T4 DNA ligase (400,000 units/mL), and ultrapure H₂O to 15 μL. The ligation reaction was incubated overnight at 16° C. The ligation reaction was then stored in the test tube at −20° C. until further use.

Heat-shock transformation: A standard heat-shock transformation was done by mixing in an ice-cold test tube 1-5 μL DNA mixture and 50 μL chemically competent Escherichia coli DH5a thawed on ice. The test tube was incubated 30 min on ice, followed by incubation for 45 sec at 42° C., and finally on ice for 5 min. To the test tube was added 800 μL sterile SOC medium. The test tube was then incubated at 37° C. under agitation to allow for cell recovery. The bacterial cells were pelleted by centrifugation at 1,000 g for 3 min at room temperature, and 750 μL of the resulting supernatant was removed. The remaining supernatant was used to resuspend the cell pellet. The suspension was dispensed and plated on a LB-agar plate supplemented with kanamycin (50 mg/L). The LB-agar plate was placed for overnight at 37° C. to culture the transformed cells.

Expression vector amplification: A single colony was picked and grown in LB medium supplemented with kanamycin (50 mg/L) for overnight at 37° C. under agitation. The cell culture was pelleted by centrifugation at 3,500 g for 10 min at room temperature. The resulting supernatant was discarded and the remaining cell pellet kept. The plasmid fraction was purified from the cells using a GeneJET Plasmid Miniprep Kit and 50 μL elution. The resulting expression vector preparation was checked by measuring the absorbance from 220 nm to 350 nm and then stored at −20° C. until further use.

### Preparation of Expression Vectors:

pET28a(+)-sfGFP: Gene insert Gene[sfGFP](SEQ ID NO.: 1) was restriction digested with NcoI-HF and XhoI, purified, and used in a ligation with vector pET28a(+) (SEQ ID NO.: 3) digested with the same restriction enzymes, purified and dephosphorylated.

pET28a(+)-SUMO-H₃KH₆: A PCR was performed using DNA template pNIC28-StrepTEVGlyHisSUMO (SEQ ID NO.: 4), and the primer set FP-SUMO-H₃KH₆ (SEQ ID NO.: 6), and RP-SUMO-H₃KH₆ (SEQ ID NO.: 7). The purified PCR product was restriction digested with NcoI-HF and BamHI, repurified, and used in a ligation with vector pET28a(+)-4CL2-H₃KH₆ (SEQ ID NO.: 8) digested with the same restriction enzymes, purified and dephosphorylated.

pET28a(+)-sfGFP-H₃KH₆: A PCR was performed using DNA template pET28a(+)-sfGFP, and the primer set T7 primer (SEQ ID NO.: 9), and RP-sfGFP-H₃KH₆ (SEQ ID NO.: 10). The purified PCR product was restriction digested with NcoI-HF and BamHI, repurified, and used in a ligation with vector pET28a(+)-4CL2-H₃KH₆ (SEQ ID NO.: 8) digested with the same restriction enzymes, purified and dephosphorylated.

pET28a(+)-MBP-H₃KH₆: A PCR was performed using DNA template pET28a(+)-MBPstar-TEV[54-237](SEQ ID NO.: 11), and the primer set FP-MBP-H₃KH₆ (SEQ ID NO.: 13), and RP-MBP-H₃KH₆ (SEQ ID NO.: 14). The purified PCR product was restriction digested with NcoI-HF and BamHI, repurified, and used in a ligation with vector pET28a(+)-4CL2-H₃KH₆ (SEQ ID NO.: 8) digested with the same restriction enzymes, purified and dephosphorylated.

pET28a(+)-SUMO-KH₆: The two complementary single-stranded DNA oligoes Forward DNA oligo KH₆ (SEQ ID NO.: 15) and Reverse DNA oligo KH₆ (SEQ ID NO.: 16) were mixed in equal ratios (50 μM), heated to 95° C. for 10 min and allowed to cool to room temperature resulting in a DNA duplex with BamHI and XhoI overhang ends. Expression vector pET28a(+)-SUMO-H₃KH₆, was digested with the restriction enzymes BamHI-HF and XhoI, purified and dephosphorylated, before being used in a ligation reaction with the prepared DNA duplex.

pET28a(+)-sfGFP-KH₆: The two complementary single-stranded DNA oligoes Forward DNA oligo KH₆ (SEQ ID NO.: 15) and Reverse DNA oligo KH₆ (SEQ ID NO.: 16) were mixed in equal ratios (50 μM), heated to 95° C. for 10 min and allowed to cool to room temperature resulting in a DNA duplex with BamHI and XhoI overhang ends. Expression vector pET28a(+)-sfGFP-H₃KH₆, was digested with the restriction enzymes BamHI-HF and XhoI, purified and dephosphorylated, before being used in a ligation reaction with the prepared DNA duplex.

pET28a(+)-MBP-KH₆: The two complementary single-stranded DNA oligoes Forward DNA oligo KH₆ (SEQ ID NO.: 15) and Reverse DNA oligo KH₆ (SEQ ID NO.: 16) were mixed in equal ratios (50 μM), heated to 95° C. for 10 min and allowed to cool to room temperature resulting in a DNA duplex with BamHI and XhoI overhang ends. Expression vector pET28a(+)-MBP-H₃KH₆, was digested with the restriction enzymes BamHI-HF and XhoI, purified and dephosphorylated, before being used in a ligation reaction with the prepared DNA duplex.

1.2 Expression and Purification of C-Terminally Tagged Proteins and Tag-Free sfGFP

Tagged protein production: E. coli BL21[DE3] cells (Invitrogen) were transformed with the expression vector encoding the respective C-terminally tagged protein construct, by standard heat-shock. A culture was grown in LB medium supplemented with 50 mg/L kanamycin and protein expression was induced with 1 mM IPTG for 4 hr at 30° C. The culture was centrifuged (10,000 g) for 10 min at 4° C. The resulting supernatant was discarded, and the cell pellet resuspended in 10 mL ice-cold aqueous 50 mM NaH₂PO₄ (pH 7.5), 300 mM NaCl, 20 mM imidazole, supplemented with EDTA-free protease inhibitor cocktail (Roche). The cell suspension was subjected to sonication in an ice-bath (48 cycles of 5 s at 1.5 W, 25 s off). The resulting lysate was high-speed centrifuged (20,000 g) for 20 min at 4° C. The cleared lysate was applied to Ni-NTA agarose (Thermo Fisher Scientific) and washed with ice-cold aqueous 50 mM NaH₂PO₄ (pH 7.5), 150 mM NaCl, 20 mM imidazole, before being eluted with aqueous 50 mM NaH₂PO₄ (pH 7.5), 150 mM NaCl, 1 M imidazole. The protein was further purified by size-exclusion chromatography on an AKTA™ pure system equipped with a Superdex 75 increase 10/300 GL column (GE Healthcare) with aqueous 50 mM NaH₂PO₄ (pH 7.5), 150 mM NaCl as eluent. Fractions containing the protein (as confirmed by SDS-PAGE analysis) were pooled and concentrated. The protein concentration was determined by measuring absorbance at 280 nm on a Nanodrop 2000 (Thermo Scientific). The purified protein was kept at −20° C. until further use.

sfGFP production: E. coli BL21[DE3] cells (Invitrogen) were transformed with the expression vector encoding the tag-free sfGFP protein construct by standard heat-shock. A culture was grown in LB medium supplemented with 50 mg/L kanamycin and protein expression was induced with 1 mM IPTG for 20 hr at 30° C., which led to secretion of the protein into the culture supernatant. The culture was high-speed centrifuged (20,000 g) for 20 min at 4° C. The resulting supernatant was kept, and 15 mL concentrated and washed with aqueous 50 mM NaH₂PO₄ (pH 7.5), 150 mM NaCl. The protein was further purified by size-exclusion chromatography on an AKTA™ pure system equipped with a Superdex 75 increase 10/300 GL column (GE Healthcare) with aqueous 50 mM NaH₂PO₄ (pH 7.5), 150 mM NaCl as eluent. Fractions containing the protein (as confirmed by SDS-PAGE analysis) were pooled and concentrated. The protein concentration was determined by measuring absorbance at 280 nm on a Nanodrop 2000 (Thermo Scientific). The purified protein was kept at −20° C. until further use.

### 1.3 C-Terminal Tag Removal by Proteolytic Cleavage

Control samples of tag-free SUMO and MBP were generated by digesting the Lys-His tagged protein with biotinylated thrombin (Novagen) or Razor™ TEV protease (Biomol GmbH), respectively, for overnight at room temperature. Tag-free sfGFP was produced as described under 1.2.

### 1.4 Protein Characteristics

Table 3 provides an overview of the protein characteristics of the expressed proteins having C-terminal KH₆ or H₃KH₆ tags as well as the tag-free counterparts.

TABLE 3
Protein characteristics

| | SEQ | | | | | ε | |
|---|---|---|---|---|---|---|---|
| | ID | | | | | (280 | Abs |
| Protein | NO. | Composition | #NH2* | Mr (cal) | Mr (obs) | nm) | 0.1% |
| SUMO^{a} | 5 | C₅₂₀H₈₃₆N₁₄₆O₁₆₇S₃ | 10 | 11,901.3 | 11,901.0 | 2980 | 0.250 |
| SUMO-KH₆ | 35 | C₅₆₇H₈₉₈N₁₆₈O₁₇₇S₃ | 11 | 12,996.6 | 12,995.9 | 2980 | 0.229 |
| SUMO-H₃KH₆ | 37 | C₅₈₅H₉₁₉N₁₇₇O₁₈₀S₃ | 11 | 13,407.9 | 13,407.9 | 2980 | 0.222 |
| sfGFP | 2 | C₁₁₈₉H₁₈₄₂N₃₂₂O₃₆₁S₆ | 21 | 26,614.0 | 26,612.9 | 18,910 | 0.710 |
| sfGFP-KH₆ | 39 | C₁₂₅₈H₁₉₄₁N₃₅₁O₃₇₅S₆ | 22 | 28,174.8 | 28,174.9 | 18,910 | 0.671 |
| sfGFP-H₃KH₆ | 41 | C₁₂₇₆H₁₉₆₂N₃₆₀O₃₇₈S₆ | 22 | 28,585.9 | 28,586.0 | 18,910 | 0.661 |
| MBP^{b} | 12 | C₁₉₃₂H₂₉₇₀N₄₉₆O₅₈₈S₇ | 36 | 42,777.8 | 42,778.0 | 67,840 | 1.586 |
| MBP-KH₆ | 43 | C₁₉₈₇H₃₀₄₅N₅₂₁O₆₀2S₇ | 37 | 44,088.6 | 44,088.9 | 67,840 | 1.539 |
| MBP-H₃KH₆ | 45 | C₂₀₀₅H₃₀₆₆N₅₃₀O₆₀₅S₇ | 37 | 44,499.5 | 44,500.5 | 67,840 | 1.524 |

*number of free amines in the protein (N-terminal α-amine and lysine ε-amines).
** determined by ExPASy¹³.
^{a}product from a thrombin digest removing the Lys-His tag.
^{b}product from a TEV protease digest removing the Lys-His tag.

### 1.5 Acylation Reactions

The acylation reaction was carried out by mixing in a test tube 1 vol. acylation reagent (compound 1: 4-methoxyphenyl ester) in DMSO to 11 vol. ice-cold aqueous solution of 32 μM protein, 50 mM NaH₂PO₄ (pH 7.5), 150 mM NaCl, 1.1 mM EDTA, 8% DMSO. After thorough mixing of the sample, the test tube was briefly spun down and then incubated for 16 hours at 4° C. For negative controls, 1 vol. DMSO was added instead of 1 in DMSO.

### 1.6 Mass Spectrometry Analysis

For mass spectrometric analysis 1 vol. acylation reaction was diluted 3-fold with 2 vol. ultrapure H₂O. The sample was applied to ESI-TOF LC-MS with a 5 μL injection volume. Data analysis was done as described: 1) An integration was performed in Bruker Compass DataAnalysis over the resulting total ion count of the protein in the mass spectrum. 2) The extracted MS trace was deconvoluted using the default settings of Maximum Entropy. 3) The deconvoluted spectrum was normalized to the highest intensity value and further integration was done in Origin 2019 for the peaks corresponding to starting material (SM), the +1 modification adduct (1 mod.), and the +2 modification adduct (2 mod.). Percentages were calculated as the ratio of the individual peaks divided by the sum of the 3 peaks times 100%.

### 1.7 Fluorescence Labeling

Protein carrying azide functionalization was labelled with fluorescence dye (compound 2: alkyne cyanine dye 718) as carried out by mixing in a test tube test 2 vol. acylation reaction, 2 vol. 1.5 mM MgSO₄, 1.5 vol. 10 mM 2 in DMSO, 2 vol. aqueous solution of 5 mM CuSO₄ with ligand 25 mM Tris(3-hydroxypropyltriazolylmethyl)amine, and 2.5 vol. 20 mM sodium ascorbate. The reaction was incubated for 1 hour at room temperature before being applied to SDS-PAGE. In-gel fluorescence was measured on a Typhoon™ FLA 7000 using the Cy5 channel.

### 1.8 Results

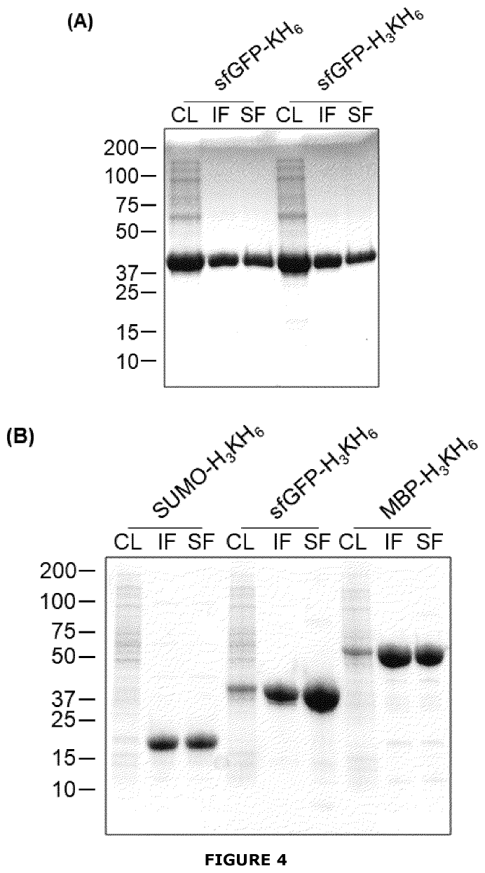

Both tags (KH₆ and H₃KH₆) facilitated purification by immobilized metal affinity chromatography (FIG. 4).

The tagged proteins were compared with tag-free versions in reactions using 4-methoxy phenyl ester 1 as acylating reagent. Mass spectrometric analysis confirmed that the tagged proteins reacted with 1 (Table 4).

An increase in the degree of acylation was observed with the KH₆ tagged proteins (5.3-fold to 34-fold) and the H₃KH₆ tagged proteins (6.3-fold to 56-fold), compared to the tag-free proteins; suggesting that the two tags promoted acylation of the proteins.

An increase in the degree of acylation was observed with the H₃KH₆ tag for all three proteins tested (1.2-fold to 1.4-fold), compared to the KH₆ tag; suggesting that the three additional histidine residues and/or positioning the target lysine residue in between histidine residues were beneficial for the reaction.

In general, diacylation (2 mod.) was observed for tagged proteins, but at low levels comparable to the non-specific labeling (1 mod.) that occurs with the tag-free variants.

TABLE 4
Acylation percentage of c-terminal tagged proteins

| | | |
|---|---|---|
| Protein | 1 mod. (%)* | 2 mod. (%)* |

| sfGFP | 7 | (±1) | 0 | |
|---|---|---|---|---|
| sfGFP-KH₆ | 43 | (±6) | 9 | (±2) |
| sfGFP-H₃KH₆ | 55 | (±7) | 17 | (±4) |
| SUMO | 9 | (±3) | 0 | |
| SUMO-KH₆ | 42 | (±5) | 6 | (±3) |
| SUMO-H₃KH₆ | 51 | (±7) | 6 | (±4) |

| | | |
|---|---|---|
| MBP | 0 | 0 |

| | | | | |
|---|---|---|---|---|
| MBP-KH₆ | 34 | (±6) | 2 | (±1) |
| MBP-H₃KH₆ | 49 | (±5) | 7 | (±3) |

Conversion of Lys-His tagged proteins and corresponding tag-free versions (29 μM) after treatment with 20 equivalents of 4-methoxy phenyl ester 1 in an aqueous solution of 50 mM NaH₂PO₄, 150 mM NaCl, 1.0 mM EDTA, pH 7.5, 8% DMSO for 16 hours at 4° C. Standard deviations are based on triplicate measurements.
*The +1 modification adduct (1 mod.) and the +2 modification adduct (2 mod.) percentages were calculated as the ratio of the individual peaks divided by the sum of the 3 peaks times 100%.

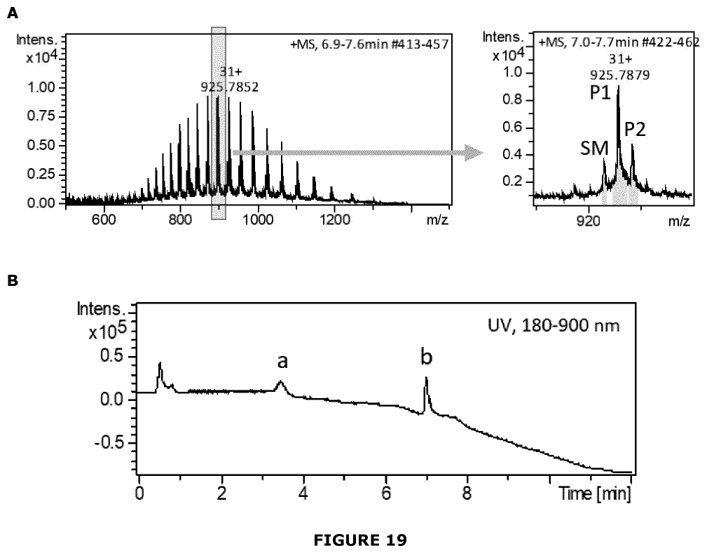

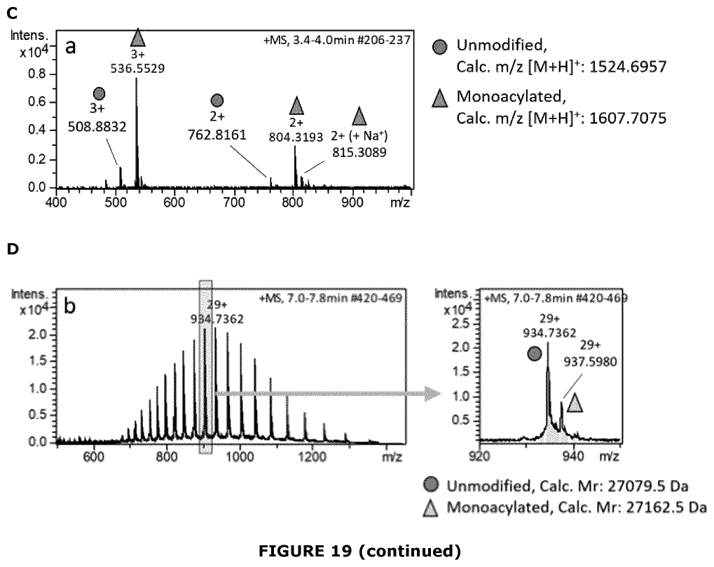

Thrombin-mediated cleavage of the H₃KH₆ tag from acylated sfGFP-H₃KH₆ followed by MS analysis of both the cleaved peptide and the truncated protein proved that the acylation had taken place on the tag (FIG. 19).

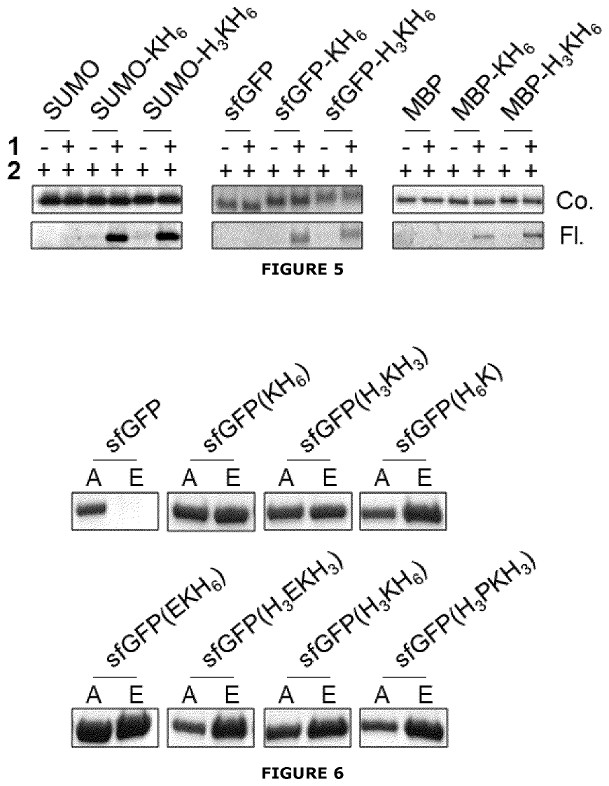

Introduction of the azide group (by reaction with the acylating reagent) facilitated conjugation of the alkyne-functionalized cyanine dye 2, as demonstrated by fluorescence labeling and gel electrophoresis (FIG. 5).

### Example 2: Site-Selective Acylation of sfGFP with Lys-His Tag Inserted Internally in a Loop Structure of the Protein

7 different small peptide tags: KHHHHHH (KH₆) (SEQ ID NO. 62), HHHKHHH (H₃KH₃) (SEQ ID NO.: 64), HHHHHHK (H₆K) (SEQ ID NO.: 65), EKHHHHHH (EKH₆) (SEQ ID NO.: 66), HHHEKHHH (H₃EKH₃) (SEQ ID NO.: 67), HHHKHHHHHH (H₃KH₆) (SEQ ID NO.: 63), HHHPKHHH (H₃PKH₃) (SEQ ID NO.: 68) were introduced into the loop connecting [3-strands β10 and β11 in the protein super-folder green-fluorescent protein (sfGFP) (FIG. 2B).

### 2.1 Preparing Vector Constructs Comprising Internally Tagged Proteins

DNA restriction digest, DNA dephosphorylation reaction, DNA ligation reaction, Heat-shock transformation, and Expression vector amplification were carried out as secribed in Example 1.1.

pET28a-sfGFP(H6): The gene insert Gene[sfGFP(H6)](SEQ ID NO.: 17) was treated with NcoI-HF and XhoI, purified and used in a ligation with vector pET28a(+) (SEQ ID NO.: 3) digested with the same restriction enzymes, purified and dephosphorylated.

pET15b-sfGFP(KH₆): The gene insert Gene[sfGFP(KH₆)](SEQ ID NO.: 19) was treated with NcoI-HF and BamHI-HF, purified and used in a ligation with vector pET15b (SEQ ID NO.: 21) digested with the same restriction enzymes, purified and dephosphorylated.

pET15b-sfGFP(H6K): The gene insert Gene[sfGFP(H6K)](SEQ ID NO.: 22) was treated with NcoI-HF and BamHI-HF, purified and used in a ligation with vector pET15b (SEQ ID NO.: 21) digested with the same restriction enzymes, purified and dephosphorylated.

pET28a(+)-sfGFP(H3KH₃): The two complementary single-stranded DNA oligoes DNA oligo T3A (SEQ ID NO.: 24) and DNA oligo T3B (SEQ ID NO.: 25) were mixed in equal ratios (50 μM), heated to 95° C. for 10 min and allowed to cool to room temperature resulting in a DNA duplex with NotI and PstI cutting sites. The DNA duplex was restriction digested with NotI and PstI, repurified and used in a ligation with the expression vector pET28a(+)-sfGFP(H6), digested with the restriction enzymes, purified and dephosphorylated.

pET28a(+)-sfGFP(EKH₆): The two complementary single-stranded DNA oligoes DNA oligo T5A (SEQ ID NO.: 26) and DNA oligo T5B (SEQ ID NO.: 27) were mixed in equal ratios (50 μM), heated to 95° C. for 10 min and allowed to cool to room temperature resulting in a DNA duplex with NotI and PstI cutting sites. The DNA duplex was restriction digested with NotI and PstI, repurified and used in a ligation with the expression vector pET28a(+)-sfGFP(H6), digested with the restriction enzymes, purified and dephosphorylated.

pET28a(+)-sfGFP(H₃EKH₃): The two complementary single-stranded DNA oligoes DNA oligo T6A (SEQ ID NO.: 28) and DNA oligo T6B (SEQ ID NO.: 29) were mixed in equal ratios (50 μM), heated to 95° C. for 10 min and allowed to cool to room temperature resulting in a DNA duplex with NotI and PstI cutting sites. The DNA duplex was restriction digested with NotI and PstI, repurified and used in a ligation with the expression vector pET28a(+)-sfGFP(H6), digested with the restriction enzymes, purified and dephosphorylated.

pET28a(+)-sfGFP(H₃KH₆): The two complementary single-stranded DNA oligoes DNA oligo T7A (SEQ ID NO.: 30) and DNA oligo T7B (SEQ ID NO.: 31) were mixed in equal ratios (50 μM), heated to 95° C. for 10 min and allowed to cool to room temperature resulting in a DNA duplex with NotI and PstI cutting sites. The DNA duplex was restriction digested with NotI and PstI, repurified and used in a ligation with the expression vector pET28a(+)-sfGFP(H6), digested with the restriction enzymes, purified and dephosphorylated.

pET28a(+)-sfGFP(H₃PKH₃): The two complementary single-stranded DNA oligoes DNA oligo T8A (SEQ ID NO.: 32) and DNA oligo T8B (SEQ ID NO.: 33) were mixed in equal ratios (50 μM), heated to 95° C. for 10 min and allowed to cool to room temperature resulting in a DNA duplex with NotI and PstI cutting sites. The DNA duplex was restriction digested with NotI and PstI, repurified and used in a ligation with the expression vector pET28a(+)-sfGFP(H6), digested with the restriction enzymes, purified and dephosphorylated.

2.2 Expression and Purification of sfGFP Loop Variants

Tagged protein production: E. coli BL21[DE3] cells (Invitrogen) were transformed with the expression vector encoding the respective sfGFP loop tagged protein construct, by standard heat-shock. A culture was grown in LB medium supplemented with 100 mg/L ampicillin or 50 mL/L kanamycin for expression vectors pET15b and pET28a(+) respectively. At an OD600 of 0.5-0.7, protein expression was induced with 1 mM IPTG for 20 hr at 30° C., which led to secretion of the protein into the culture supernatant. The culture was high-speed centrifuged (20,000 g) for 20 min at 4° C. The resulting supernatant was kept, and 15 mL concentrated and washed with aqueous 50 mM NaH₂PO₄ (pH 7.5), 300 mM NaCl, 20 mM imidazole. The solution was then applied to Ni—NTA agarose (Thermo Fisher Scientific) and washed with aqueous 50 mM NaH₂PO₄ (pH 7.5), 150 mM NaCl, 20 mM imidazole, before being eluted with aqueous 50 mM NaH₂PO₄ (pH 7.5), 150 mM NaCl, 1 M imidazole. The protein was further purified by size-exclusion chromatography on an AKTA™ pure system equipped with a Superdex 75 increase 10/300 GL column (GE Healthcare) with aqueous 50 mM NaH₂PO₄ (pH 7.5), 150 mM NaCl as eluent. Fractions containing the protein (as confirmed by SDS-PAGE analysis) were pooled and concentrated. The protein concentration was determined by measuring absorbance at 280 nm on a Nanodrop 2000 (Thermo Scientific). The purified protein was kept at −20° C. until further use.

sfGFP production: E. coli BL21[DE3] cells (Invitrogen) were transformed with the expression vector encoding the tag-free sfGFP protein construct by standard heat-shock. A culture was grown in LB medium supplemented with 50 mg/L kanamycin and protein expression was induced with 1 mM IPTG for 20 hr at 30° C., which led to secretion of the protein into the culture supernatant. The culture was high-speed centrifuged (20,000 g) for 20 min at 4° C. The resulting supernatant was kept, and 15 mL concentrated and washed with aqueous 50 mM NaH₂PO₄ (pH 7.5), 150 mM NaCl. The protein was further purified by size-exclusion chromatography on an AKTA™ pure system equipped with a Superdex 75 increase 10/300 GL column (GE Healthcare) with aqueous 50 mM NaH₂PO₄ (pH 7.5), 150 mM NaCl as eluent. Fractions containing the protein (as confirmed by SDS-PAGE analysis) were pooled and concentrated. The protein concentration was determined by measuring absorbance at 280 nm on a Nanodrop 2000 (Thermo Scientific). The purified protein was kept at −20° C. until further use.

### 2.3 Protein Characteristics

Table 5 provides an overview of protein characteristics of the expressed sfGFP proteins having internal His-Lys tags in the specified loop region.

TABLE 5
Protein characteristics

| | SEQ | | | | | ε | |
|---|---|---|---|---|---|---|---|
| | ID | | | | | (280 | Abs |
| Protein | NO. | Composition | #NH2* | Mr (cal) | Mr (obs) | nm) | 0.1% |
| sfGFP(KH6) | 20 | C₁₂₄₆H₁₉₁₉N₃₄₇O₃₇₃S₆ | 22 | 27,920.4 | 27,920.7 | 18,910 | 0.677 |
| sfGFP(H₆K) | 23 | C₁₂₄₆H₁₉₁₉N₃₄₇O₃₇₃S₆ | 22 | 27,920.4 | 27,920.6 | 18,910 | 0.677 |
| sfGFP(EKH₆) | 47 | C₁₂₅₄H₁₉₃₃N₃₄₉O₃₇₇S₆ | 22 | 28,120.6 | 28,120.7 | 18,910 | 0.672 |
| sfGFP(H₃KH₃) | 49 | C₁₂₄₉H₁₉₂₆N₃₄₈O₃₇₄S₆ | 22 | 27,991.5 | 27,991.6 | 18,910 | 0.675 |
| sfGFP(H₃EKH₃) | 51 | C₁₂₅₄H₁₉₃₃N₃₄₉O₃₇₇S₆ | 22 | 28,120.6 | 28,120.3 | 18,910 | 0.672 |
| sfGFP(H₃PKH₃) | 53 | C₁₂₅₄H₁₉₃₃N₃₄₉O₃₇₅S₆ | 22 | 28,088.6 | 28,088.9 | 18,910 | 0.673 |
| sfGFP(H₃KH₆) | 55 | C₁₂₆₉H₁₉₄₇N₃₅₇O₃₇₉S₆ | 22 | 28,461.0 | 28,461.2 | 18,910 | 0.664 |

*number of free amines in the protein (N-terminal α-amine and lysine ε-amines).
** determined by ExPASy¹³.

The intrinsic absorbance properties of sfGFP allowed assessment of the impact of the Lys-His tags on the protein fold¹⁴. All sfGFP versions were obtained in similar yields and had absorbance characteristics matching those of the wildtype protein (Table 5), with all versions observed to bind to Ni-NTA resin (data not shown).

### 2.4 Acylation Reactions, Mass Spectrometry Analysis, and Fluorescence Labeling

The acylation reaction was carried out as described in Example 1.5. Mass spectrometric analysis was carried out as described in Example 1.6. Fluorescence labeling was carried out as described in Example 1.7.

### 2.5 Results

All tags (KH₆, H₃KH₆, H₃KH₃, H₆K, EKH₆, H₃EKH₃, H₃KH₆, and H₃PKH₃) facilitated purification by immobilized metal affinity chromatography (FIG. 6).

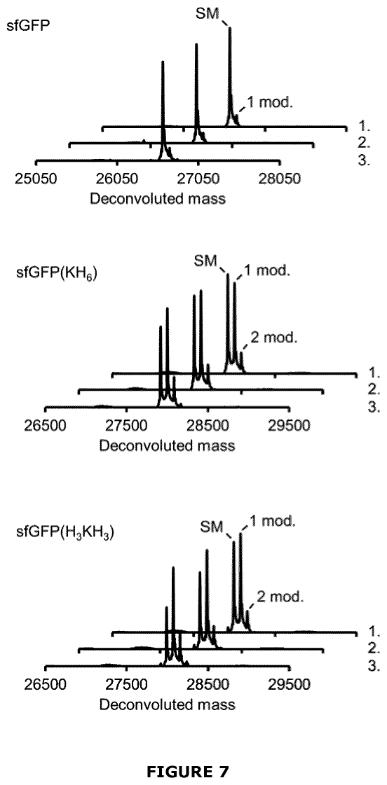

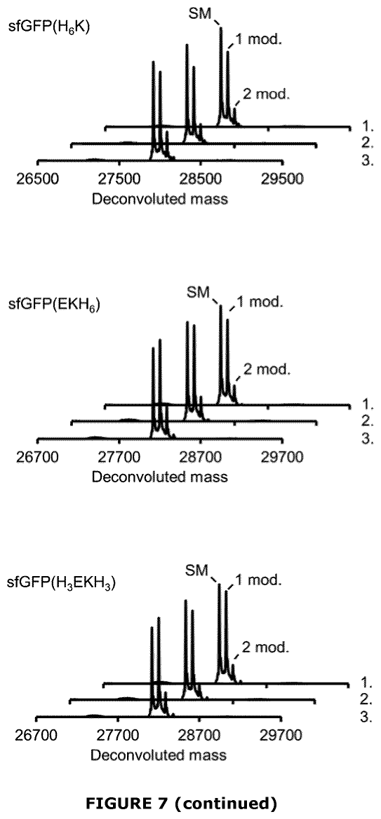

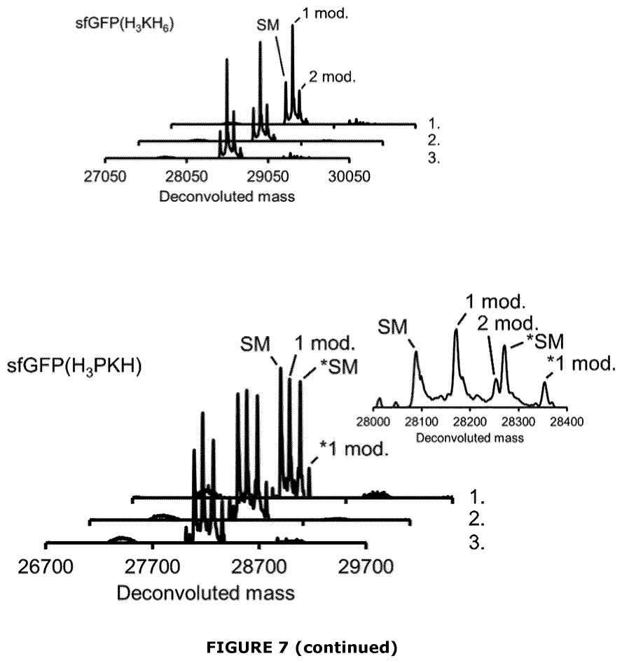

Compared to tag-free sfGFP, all of the Lys-His tags provided ample acylation (FIG. 7 and Table 6). The effect of the position of the Lys-residue within the tag was investigated as well as the effect of the presence of a residue other than histidine, specifically glutamate (Glu) having a negatively charged side chain, and proline (Pro) which may change the conformation of the tag.

It was found that: (1) The presence of His residues on both sides of the reactive Lys (KH₆ and H₆K vs. H₃KH₃) (experiment performed in triplicate) had only a minor effect (1.1-fold increase in the degree of acylation). (2) The presence of an additional residue in between the His residues and the reactive Lys residue (H₃EKH₃ and H₃PKH₃ vs. H₃KH₃) had only a minor effect (1.1-fold reduction in acylation efficiency).

TABLE 6
Acylation percentage of proteins tagged in a loop

| Protein | 1 mod. (%)* | 2 mod. (%)* |
|---|---|---|
| sfGFP | 10 (±1) | 0 |
| sfGFP(KH₆) | 46 (±1) | 12 (±2) |
| sfGFP(H₃KH₃) | 49 (±3) | 14 (±3) |
| sfGFP(H₆K) | 43 (±3) | 11 (±2) |
| sfGFP(EKH₆) | 44 (±1) | 12 (±2) |
| sfGFP(H₃EKH₃) | 45 (±1) | 10 (±2) |
| sfGFP(H₃KH₆) | 59 (±1) | 23 (±4) |
| sfGFP(H₃PKH₃) | 45 (±2) | 9 (±2) |

*Conversion of Lys-His tagged proteins and corresponding tag-free versions (29 μM) after treatment with 40 equivalents of 4-methoxy phenyl ester 1 in an aqueous solution of 50 mM NaH₂PO₄, 150 mM NaCl, 1.0 mM EDTA, pH 7.5, 8% DMSO for 16 hours at 4° C. Standard deviations are based on triplicate measurements.

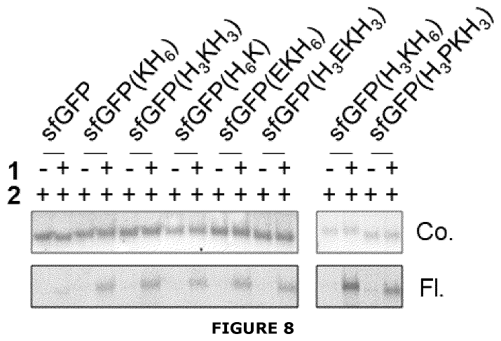

Introduction of the azide group (by reaction with the acylating reagent) facilitated conjugation of the alkyne-functionalized cyanine dye 2, as demonstrated by fluorescence labeling and gel electrophoresis (FIG. 8).

Comparing with the C-terminal tags (Experiment 1), the KH₆ tag displayed a slightly lower performance when placed in the loop instead of at the C-terminus of sfGFP. This might reflect the difference in conformational freedom between the restricted loop and the free terminal chain.

### Example 3: Divalent Metals Affect Acylation Efficiency

### 3.1 Acylation Reactions, Mass Spectrometry Analysis, and Fluorescence Labeling

The acylation reaction was carried out as described in Example 1.5. Mass spectrometric analysis was carried out as described in Example 1.6.

### 3.2 Results

Acylation reactions of different sfGFP tags were carried out as described in Example 2, except that EDTA was included in the reaction buffer in the acylation reactions.

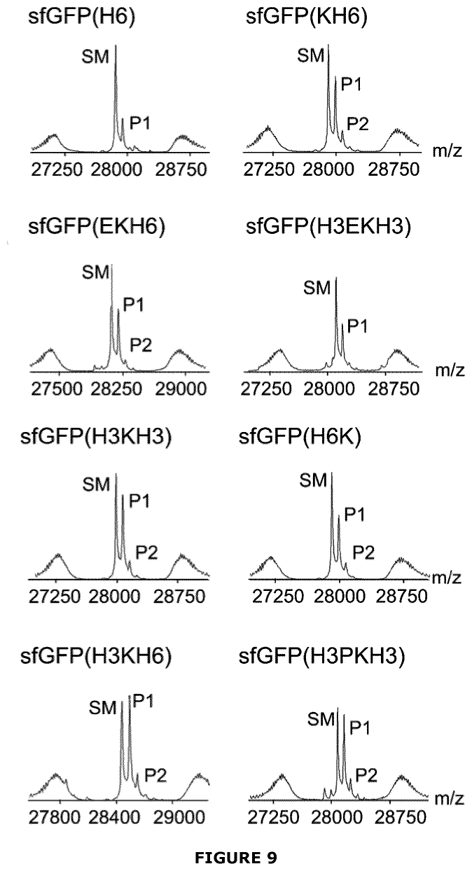

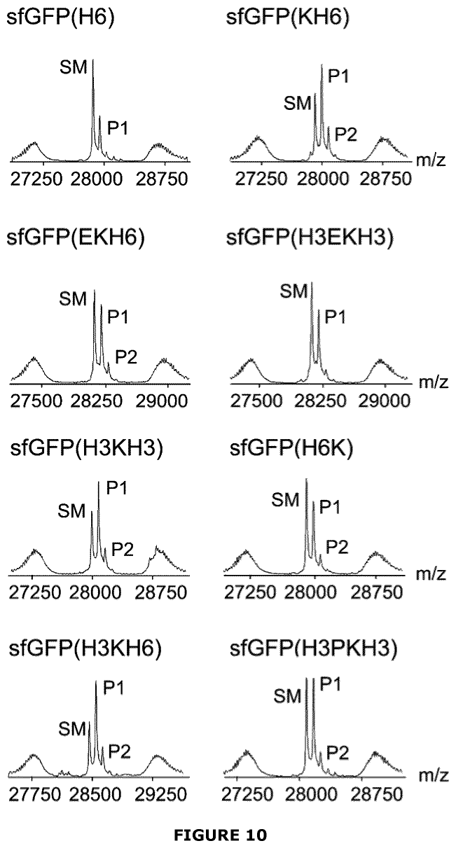

It was found that the acylation was largely affected by EDTA, suggesting that trace metals have an effect on the reactions. Without wishing to be bound by theory, it is speculated that histidine binding of metal ions confers a potential limitation on the acylation reaction, as the histidine(s) then cannot support the acylation of the lysine. FIG. 10 supports this hypothesis, as it shows an overall improvement of the acylation efficiency by addition of EDTA to the reaction buffer, compared to FIG. 9 without EDTA. EDTA binds the metal ions, thereby taking away the inhibition described above.

To further test this effect, experiments were conducted on sfGFP-KH₆ (C-terminal tag, as described in Example 1) and sfGFP(KH₆) (internal loop tag, as described in Example 2) (Table 7). Regardless of the positioning of the Lys-His tag, certain metal ions (Co²⁺, Ni²⁺, Zn²⁺, Cu²⁺) had an inhibitory effect in regards to the acylation reaction with 1. However, whereas EDTA (1 mM) was shown to be beneficial for the reaction with sfGFP(KH₆), this effect was not seen with sfGFP-KH₆, suggesting positioning of the KH₆ Lys-His tag to be important. Low concentrations of EDTA may therefore preferably be included in the reaction buffer as a precaution when the Lys-His tags are positioned within a loop, but not when positioned C-terminally. Alternatively, a two-step purification procedure can be used including a second size-exclusion chromatographic step.

TABLE 7
Acylation percentage of tagged proteins

| | Protein | | | |
|---|---|---|---|---|
| | sfGFP-KH₆ | | sfGFP(KH₆) | |
| | Additive | | | |
| | 1 mod. (%)* | 2 mod. (%)* | 1 mod. (%)* | 2 mod. (%)* |
| Mock | 47 | 11 | 33 | 5 |
| CoSO₄ | 24 | 0 | 15 | 0 |
| NiSO₄ | 14 | 0 | 14 | 0 |
| ZnCl₂ | 8 | 0 | 12 | 0 |
| CuSO₄ | 17 | 0 | 20 | 0 |
| MgSO₄ | 45 | 10 | ND | ND |
| EDTA | 45 | 9 | 44 | 13 |

*Conversion of Lys-His tagged proteins (29 μM) after treatment with 4-methoxy phenyl ester 1 in an aqueous solution of 50 mM NaH₂PO₄, 150 mM NaCl, 1.0 mM divalent metal ion or EDTA, pH 7.5, 8% DMSO for 16 hours at 4° C. In reactions with sfGFP-KH₆ and sfGFP(KH₆) was used 20 eq. and 40 eq. 1 respectively.

### Example 4: Biotinylation of Proteins

### 4.1 Protein Biotinylation by Acylation Reactions

The acylation reaction was carried out in a test tube by addition of 1 vol. acylation reagent (compound 5: D-biotin N-hydroxysuccinimide ester; compound 6a: 4-methoxyphenyl 2-(2-(2-(D-biotinylamino)ethoxy)ethoxy)acetate; compound 6b: 3-chloro-4-methoxyphenyl 2-(2-(2-(D-biotinylamino)ethoxy)ethoxy)-acetate) in DMSO to 11 vol. ice-cold aqueous solution of 32 μM protein, 50 mM NaH₂PO₄ (pH 7.5), 150 mM NaCl, 1.1 mM EDTA. After thorough mixing of the sample, the test tube was briefly spun down and then incubated with reaction times and temperature conditions as specified in the text. For negative controls, 1 vol. DMSO was added instead of 1 in DMSO.

### 4.2 Capture of Biotinylated Protein to Streptavidin-Beads

A given acylation reaction was transferred to an Amicon Ultra-0.5 mL Centrifugal Filter unit (NMWL=3 kDa) and washed with aqueous 50 mM NaH₂PO₄ (pH 7.5), 150 mM NaCl to remove excess biotin. The sample was isolated in a new test tube to which was added beads carrying immobilized streptavidin pre-equilibrated with aqueous 50 mM NaH₂PO₄ (pH 7.5), 150 mM NaCl. The test tube was incubated overnight at room temperature under agitation to allow for capture of the protein to the beads. The beads were washed 3 times by repeating a cycle of: 1) Gently spinning the test tube, 2) Removing the resulting supernatant, and 3) Adding new aqueous 50 mM NaH₂PO₄ (pH 7.5), 150 mM NaCl. Finally, the beads were suspended in aqueous 50 mM NaH₂PO₄ (pH 7.5), 150 mM NaCl and transferred to a transparent 8-well plate. Imaging was performed using a Leica DM5500 B upright wide-field microscope equipped with epifluorescence optics. Images were recorded using the GFP channel.

### 4.3 Mass Spectrometry Analysis

Mass spectrometric analysis was carried out as described in Example 1.6

### 4.4 Western Blot Analysis

Following standard procedures for gel electrophoresis, transfer and blocking (3% skimmed milk in PBST, 1 h at room temperature), the nitrocellulose membrane was incubated with streptavidin-HRP (1 μg/mL in 3% skimmed milk in PBST, 1 h at room temperature). Bound streptavidin was detected using a chemiluminescent detection reagent (Amersham ECL Prime western blotting detection reagent).

### 4.5 Results

Different biotin ester derivatives were tested to directly biotinylate proteins. The reacted samples were incubated with streptavidin-coated resin to allow modified protein to bind and the capture was determined by fluorescence microscopy.

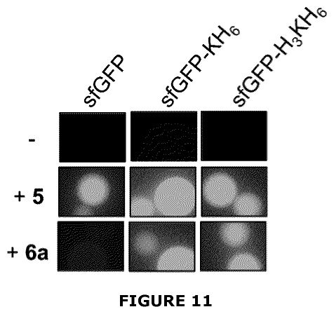

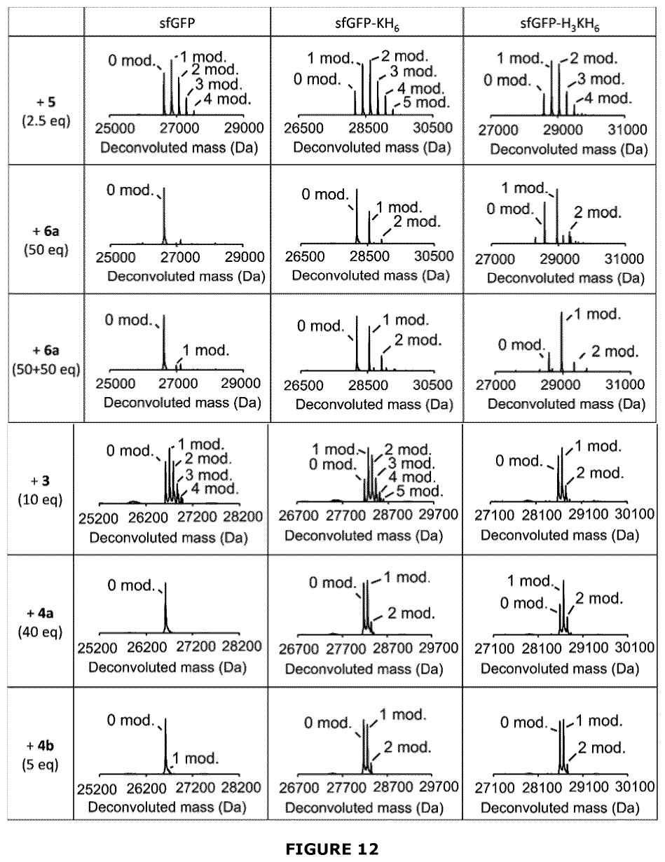

The use of biotin NHS ester 5 facilitated binding of protein to the resin in all three cases (FIG. 11). In contrast, acylation with 6a led to a significant enrichment of sfGFP-KH₆ and sfGFP-H₃KH₆, whereas the control sample with tag-free sfGFP produced only a weak fluorescence signal (FIG. 11). These observations were supported by MS data (FIG. 12), where a single addition of 50 eq. of 6a led to 31% biotinylated sfGFP-KH₆ and 63% biotinylated sfGFP-H₃KH₆ after overnight incubation at 4° C. Adding the reagent 6a twice as two portions of 50 eq. gave 47% biotinylated sfGFP-KH₆ and 90% biotinylated sfGFP-H₃KH₆ after overnight incubation at 4° C. In contrast, less than 3% of tag-free sfGFP was biotinylated under identical conditions.

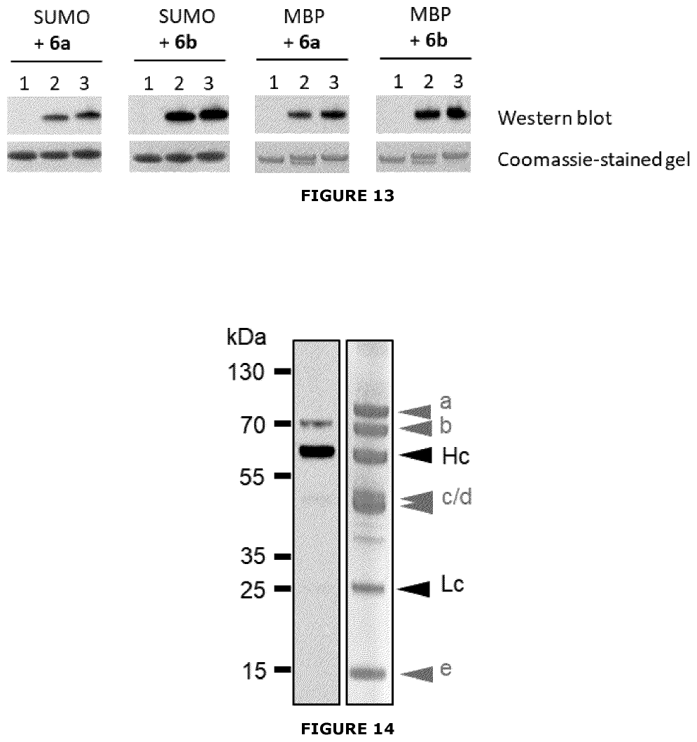

Finally, both 6a and 6b were tested on tag-free as well as KH₆ and H₃KH₆ tagged SUMO and MBP to confirm the general applicability of the biotinylation concept, and it was again found that tag-free protein remained largely unmodified in contrast to tagged protein as determined by western-blotting analysis (FIG. 13).

### Example 5: Site-Specific Acylation of Rituximab

### 5.1 Antibody Tagging

The light chain and heavy chain of the antibody Rituximab were expressed from a single mammalian expression vector (pBudCE4.1 from Thermofisher). The coding sequences of both antibody chains were synthesized by Geneart (Thermofisher). The gene encoding the light chain was under the control of the human elongation factor 1 alpha subunit promoter (pEF1a) while the gene encoding the heavy chain was under the control of the human cytomegalovirus promoter (pCMV). The tags KH₆ and H₃KH₆ were cloned in-frame at the C-terminus of the heavy chain. Standard molecular biology procedures were employed for generation of the expression vectors (SEQ ID NO.: 56 and 59, respectively).

Both antibody versions were expressed in Chinese Hamster Ovary (CHO) cells. Cells were cultured in CD CHO medium (Gibco 10743-029) supplemented with 8 mM L-glutamine (Lonza BE17-605F) and 2 mL/L of anti-clumping agent (Gibco 0010057AE), according to the Gibco guidelines. The day prior to transfection, cells were washed and cultured in exponential phase in medium not supplemented with anti-clumping agent. At the day of transfection, viable cell density was adjusted to 800,000 cells/mL in 2 L shake flasks (Corning 431143) containing 500 mL medium only supplemented with 8 mM L-glutamine. For each transfection, 500 μg plasmid was diluted in OptiPro SFM (Gibco 12309019) to a final volume of 12.5 mL. Separately, 1.5 mL FuGene HD reagent (Promega E2311) was diluted in 11 mL OptiPro SFM. The plasmid/OptiPro SFM mixture was added to the FuGENE HD/OptiPro SFM mixture and incubated at room temperature for 5 minutes and the resultant 25 mL plasmid/lipid mixture was added dropwise to the cells. Supernatants containing antibody were harvested after 72 h by centrifugation of cell culture at 1,000 g for 10 minutes and stored at −80° C. until purification.

Supernatants were thawed and loaded onto a 5-mL HiTrap MAb Select column (GE Healthcare) pre-equilibrated with five column volumes of 20 mM NaH₂PO₄ (pH 7.2) and 150 mM NaCl. The column was washed with 20 column volumes of equilibration buffer and the protein eluted with 100 mM citric acid (pH 3.0). The elution fractions were neutralized upon elution with 1 M Tris (pH 9.0) (100 μL per 500 μL elution fraction). Fractions containing the protein were pooled and diluted ten-fold in 50 mM NaH₂PO₄ (pH 7.5). As such, the protein was loaded on a 5-mL HiTrap SF FF column (GE Healthcare) pre-equilibrated with ten column volumes of 50 mM NaH₂PO₄ (pH 7.5). The column was washed with five column volumes of equilibration buffer and the protein eluted with five column volumes of 50 mM NaH₂PO₄ (pH 7.5) and 200 mM NaCl. Fractions containing the protein were pooled, concentrated using an Amicon-15 centrifugal filter device (Millipore, 30 kDa MWCO) and stored at −20° C. until further use. The protein concentration was determined by measuring absorbance at 280 nm on a Nanodrop 2000 (Thermo Scientific) using the extinction coefficient determined by ExPASy ProtParam¹³.

### 5.2 Acylation of Rituximab

Next, the tagged antibody variants were reacted with acylating reagent 1 under similar conditions as those used for sfGFP, SUMO and MBP. The buffer solution was composed of 50 mM sodium phosphate, 150 mM NaCl (pH 7.5) and 0.1 mM EDTA. The concentration of protein was 10 μM (corresponding with 20 μM of tag), and 1 vol. acylation reagent 1 (2.5 μL of a 16.8 mM stock solution in DMSO; equal to a final concentration of 1.2 mM) was added to 14.3 vol. ice-cold protein solution. The reactions were left at 4° C. overnight (approx. 20 h).

### 5.3 Mass Spec Analysis

The reacted antibodies were reduced prior to MS analysis, such that modification of the light chain and heavy chain could be assessed separately.

### 5.4 Results

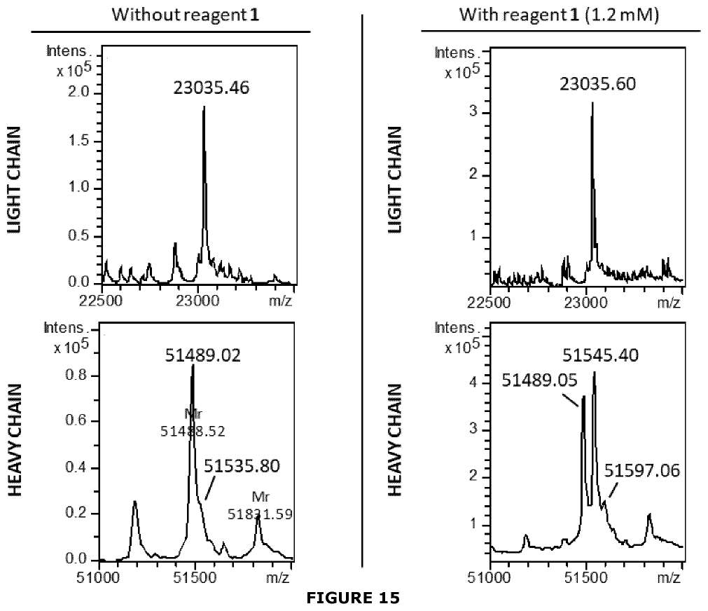

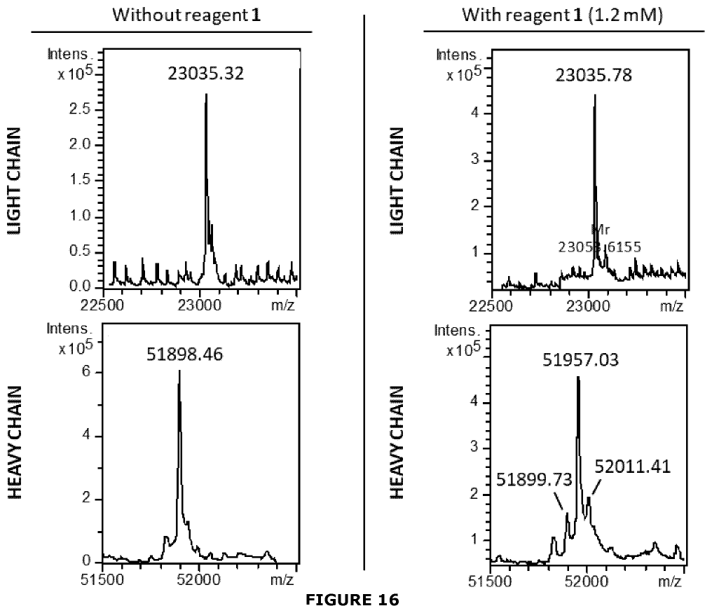

As expected, the heavy chains of both antibody versions, Rituximab-KH₆ and Rituximab-H₃KH₆, were found to be modified, whereas the light chains (no tag) displayed the same mass before and after acylation (FIGS. 15 and 16). Also for Rituximab, the acylation was more efficient when the protein was tagged with H₃KH₆ instead of KH₆ (approx. 88% vs. 72% of acylated protein).

### Example 6: Selective Labeling of Rituximab

Rituximab-H₃KH₆ was prepared as described in Example 5. Biotin derivative 6a was added to a mixture of proteins (conalbumin, bovine serum albumin, aldolase, ovalbumin and lysozyme) which further contained the Rituximab-H₃KH₆ as the only Lys-His tagged protein. The same buffer composition and acylation reaction conditions as described in Example 5 were employed. The concentration of Rituximab-H₃KH₆ was 1.5 mg/mL (equal to 10 μM) and the concentration of the untagged proteins (conalbumin, bovine serum albumin, aldolase, ovalbumin and lysozyme) varied between 0.65 to 0.8 mg/mL. The concentration of 6a in the reaction mixture was 0.5 mM. Biotinylated protein species were detected by Western blot analysis (same procedure as the Western blot analysis described in Example 4), while a gel loaded with an aliquot of the same sample but instead stained with Coomassie protein dye, displayed the relative abundance of all proteins in the reaction mixture (FIG. 14). The heavy chain (Hc) of tagged Rituximab was the most predominantly biotinylated product in the reaction mixture and only very faint bands corresponding with minor amounts of biotinylated, untagged proteins were detected (such as seen for BSA which may be explained by the presence of sites in the protein to which the acylation reagent tends to bind in a non-covalent manner. Upon formation of the protein-acylation reagent complex, one or more Lys residues in the vicinity of the binding site may react with the acylation reagent, which will thereby get covalently coupled to the protein.) Overall, the results show that the method is not restricted to pure, tagged proteins and suggests its broader applicability in more complex biological samples.

### Example 7: Effect of Number of his-Residues in the Lys-His Tag

The minimal number of His residues required for site selective acylation of lysine was assessed using Rituximab with a C-terminal KHn tag (n=1-6) on the heavy chain (single measurement). The tagged Rituximab antibodies were prepared as in to Example 5.

Experimental details: The same buffer composition and acylation reaction conditions as described in Example 5. The concentration of Rituximab-KHn was 0.95 mg/mL (equal to 6.3 μM) and the concentration of acylation reagent 1 in the reaction mixture was 0.9 mM.

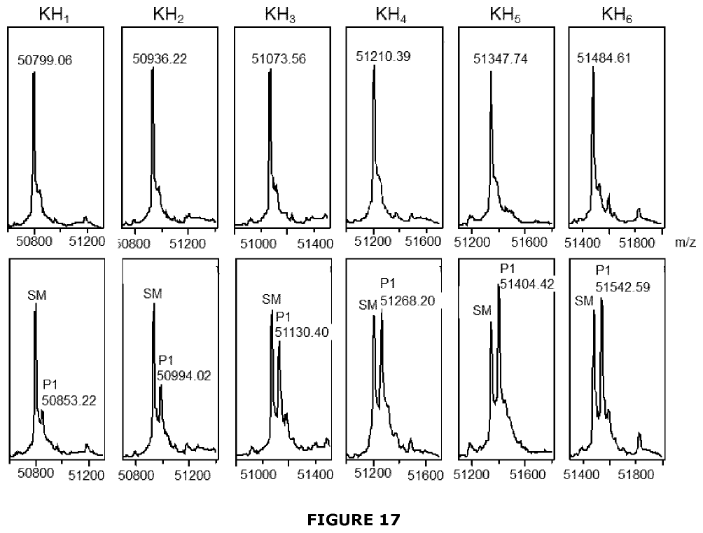

Results: With the number of His residues in the tag being 1-4, an increase in the degree of acylation was observed, while with the number of His residues in the tag being 4-6, similar degrees of acylation were obtained (FIG. 17).

### Example 8: Effect of Choice of Acylating Reagent

While the composition and positioning of the Lys-His tag was found to influence the reactivity towards phenyl ester 1, the ability to tune the reaction by the choice of acylation reagent was investigated. Different azido ester derivatives and biotin ester derivatives (Table 1) were compared in reactions with tag-free and Lys-His tagged sfGFP. In addition, it was assessed how the use of a more reactive acylation reagent affected the degree of acylation of Lys-His tagged Rituximab.

8.1 Experimental Details for Acylation of Tag-Free and Lys-His Tagged sfGFP

Protein (29 μM) was reacted with various concentrations of ester in aqueous 50 mM NaH₂PO₄, 150 mM NaCl, 1 mM EDTA, 8% DMSO (pH 7.5) at 4° C. for 16 hr. The percentages are derived from three independent experiments. Azido esters tested were 1, 3, 4a, and 4b in addition to biotin esters 5, and 6a (Table 1).

### 8.2 Experimental Details for Acylation of Rituximab-KH₄

The reactivity of reagents 1 vs. 4b on C-terminally tagged Rituximab-KH₄ was tested by using the same buffer composition and acylation reaction conditions as described in Example 5. The concentration of Rituximab-KH₄ was 1.5 mg/mL (equal to 10 μM), and the concentration of acylation reagents 1 and 4b in the reaction mixture was 1.5 mM and 0.5 mM, respectively.

### 8.3 Results

The type of acylating reagent used plays a role in the selectivity and efficiency of the acylation reaction. 4-methoxy phenyl ester derivatives are less reactive than N-hydroxysuccinimide (NHS) esters, the most commonly used compounds for protein modification. It was found that tag-free sfGFP gets modified by NHS-containing compounds 3 and 5, but not by 4-methoxy phenyl ester derivatives 4a-b and 6a (FIG. 12). Only when sfGFP comprised the Lys-His tag of the present invention, it got modified with the 4-methoxy phenyl ester derivatives. Tag-free sfGFP does get modified by compound 1 to a minor degree (7%+/−1%, at 20 eqv; Table 4); this depends largely on the excess used of the ester—the larger excess the more modification even without tag. But there is a significant increase in modification once the Lys-His tag (KH₆ or H₃KH₆) is added (Table 4).

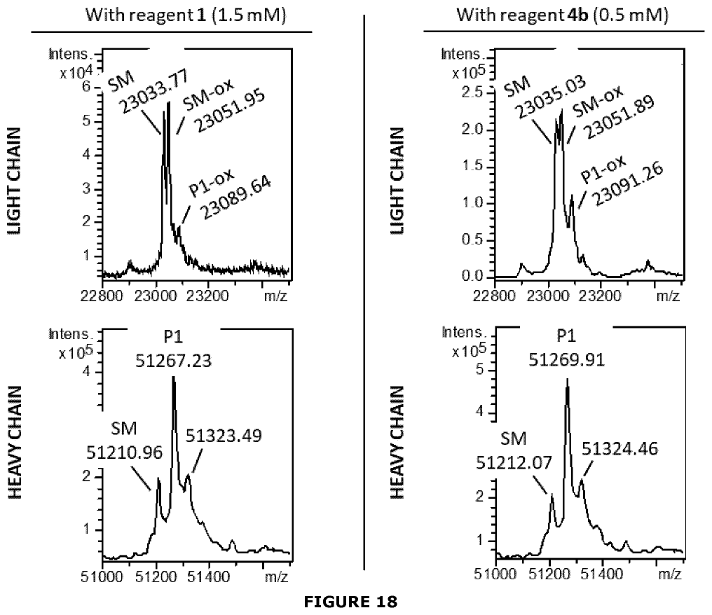

Additional substituents on the phenyl ring have an effect on the reactivity of the reagent. Compound 4a is less reactive than compound 1 and can come useful when compound 1 is found to be too reactive (that is, reacting with off-target Lys residues or the N-terminal amine) for a given protein of interest. Compound 4b is more reactive than compound 1. Accordingly, it was for Rituximab observed that the concentration of acylating reagent could be reduced 3-fold when using 4b instead of 1. Compared to compound 1, this lower concentration of compound 4b provided a similar degree of acylation of the heavy chain of Rituximab C-terminally tagged with KH₄. However, the increased reactivity of 4b led to off-target acylation of the light chain of Rituximab (FIG. 18). Hence, 4b is more reactive, but less selective.

### Example 9: Synthesis of Acylating Reagents

### 9.1 Synthesis of 4-methoxyphenyl 2-azidoacetate (1)

4-Methoxyphenol (1.03 g, 8.3 mmol) and 4-dimethylaminopyridine (100 mg, 0.8 mmol) were dissolved in CH₂Cl₂ (50 mL) under stirring. 2-Azidoacetic acid (1.01 g, 10.0 mmol) was added. Then, N,N′-diisopropylcarbodiimide (1.57 mL, 10.0 mmol) was added dropwise over a period of 10 min. The reaction mixture turned yellow to brown over 30 min. Stirring was continued for 1.5 h. Then, Celite (10 g) was added to the reaction mixture, and the solvent was removed by rotary evaporation. The resulting brown Celite adsorbate was loaded on top of a pre-conditioned (ethyl acetate/heptane 1:39) column for vacuum liquid chromatography (H×D=7×5 cm; silica gel 60 (0.015-0.040 mm)), and the product was eluted using a gradient of ethyl acetate/heptane (1:39→1:3). Fractions containing the pure product were combined and concentrated by rotary evaporation. The product was dried in vacuo to yield 1.63 g (95%) of 1 as a colourless oil. ¹H NMR (300 MHz, CDCl₃), δ 7.12-7.02 (m, 2H, 2×H—Ar), 6.97-6.87 (m, 2H, 2×H—Ar), 4.12 (s, 2H, CH₂), 3.82 (s, 3H, CH₃). ¹³C NMR (75 MHz, CDCl₃) δ 167.29 (C═O), 157.73 (C—Ar), 143.69 (C—Ar), 122.05 (2×C-Ar), 114.67 (2×C-Ar), 55.68 (CH₃), 50.49 (CH₂). HR-MS (Q-TOF): m/z calcd for chemical formula C₉H₉N₃O₃: 207.0644; found: [M+Na]⁺ 230.0503.

### 9.2 Synthesis of 3,4-dimethoxyphenyl 2-azidoacetate (4a)

0.415 mmol of 3,4-dimethoxyphenol and 0.04 mmol of 4-dimethylaminopyridine (DMAP) were dissolved in 5 mL of CH₂Cl₂ under stirring. 0.5 mmol of 2-azidoacetic acid was added and thereafter 0.5 mmol of N,N′-diisopropylcarbodiimide was added to the solution, dropwise, over a period of 5 minutes. The reaction mixtures changed colour over 30 minutes from brown to dark brown. Stirring was continued for 1.5 h. Then, 0.5 g of Celite was added to the reaction mixture and the solvent was removed by rotary evaporation. The resulting Celite adsorbate was loaded on top of a pre-conditioned (1:39 ethyl acetate/heptane) column for vacuum liquid chromatography (H×D=7×5 cm; silica gel 60 (0.015-0.040 mm)). The product was eluted with an ethyl acetate/heptane gradient of 1:39→3:5. Fractions containing the pure product were checked by TLC, and were then combined and concentrated by rotary evaporation. The product was dried in vacuo to yield 180 mg (89%) of 4a as a brown oil. ¹H NMR (500 MHz, CDCl₃): δ 6.85 (d, J=8.5 Hz, 1H; H—Ar), 6.72-6.67 (m, 2H; 2×H—Ar), 4.12 (s, 2H; CH₂), 3.87 (d, J=5.7 Hz, 6H; 2×CH₃). ¹³C NMR (125 MHz, CDCl₃): δ 167.33 (C═O), 149.64 (C—Ar), 147.42 (C—Ar), 143.84 (C—Ar), 112.64 (C—Ar), 111.31 (C—Ar), 105.45 (C—Ar), 56.34 (CH₃), 56.19 (CH₃), 50.59 (CH₂). HR-MS (ESI): m/z calculated for chemical formula C₁₀H₁₁N₃O₄: 237.0750; found [M+H]⁺ 238.0810.

### 9.3 Synthesis of 3-chloro-4-methoxyphenyl 2-azidoacetate (4b)

0.415 mmol of 3-chloro-4-methoxyphenol and 0.04 mmol of 4-dimethylaminopyridine (DMAP) were dissolved in 5 mL of CH₂Cl₂ under stirring. 0.5 mmol of 2-azidoacetic acid was added and thereafter 0.5 mmol of N,N′-diisopropylcarbodiimide was added to the solution, dropwise, over a period of 5 minutes. The reaction mixtures changed colour over 30 minutes from yellow to brown. Stirring was continued for 1.5 h. Then, 0.5 g of Celite was added to the reaction mixture and the solvent was removed by rotary evaporation. The resulting Celite adsorbate was loaded on top of a pre-conditioned (1:39 ethyl acetate/heptane) column for vacuum liquid chromatography (H×D=7×5 cm; silica gel 60 (0.015-0.040 mm)). The product was eluted with an ethyl acetate/heptane gradient of 1:39→1:3. Fractions containing the pure product were checked by TLC, and were then combined and concentrated by rotary evaporation. The product was dried in vacuo to yield 48 mg (54%) of 4b as a white solid. ¹H NMR (500 MHz, CDCl₃): δ 7.21 (d, J=2.5 Hz, 1H; H—Ar), 7.03 (dd, J=2.9 and 9.0 Hz, 1H; H—Ar), 6.92 (d, J=9.0 Hz, 1H; H—Ar), 4.11 (s, 2H; CH₂), 3.90 (s, 3H; CH₃). ¹³C NMR (125 MHz, CDCl₃): δ 167.06 (C═O), 153.56 (C—Ar), 143.32 (C—Ar), 123.44 (C—Ar), 123.04 (C—Ar), 120.40 (C—Ar), 112.27 (C—Ar), 56.63 (CH₃), 50.49 (CH₂). HR-MS (ESI): m/z calculated for chemical formula C₉H₈ClN₃O₃: 241.0254; found [M+Na]⁺ 264.0121.

### 9.4 Synthesis of 4-methoxyphenyl 2-(2-(2-(D-biotinylamino)ethoxy)ethoxy)acetate (6a)

A solution of 8-(9-fluorenylmethyloxycarbonylamino)-3,6-dioxaoctanoic acid (1.93 g, 5 mmol, 5 equiv.) in dry CH₂Cl₂ (4 mL) was added to 2-chlorotrityl chloride polystyrene resin (1 g, 1 mmol/g, 100-200 mesh, 1% DVB), and the reaction mixture was agitated for 16 h. The resin was washed with CH₂Cl₂ (5×4 mL) and N,N-dimethylformamide (DMF, 5×4 mL), and the Fmoc group was removed by treatment with 20% piperidine in DMF (4 mL) for 5 min., followed by 20% piperidine in DMF (4 mL) for 15 min. The resin was then washed with DMF (5×4 mL), and CH₂Cl₂ (5×4 mL), followed by DMF (5×4 mL). D-Biotin (150 mg, 0.6 mmol) was preactivated with HATU (190 mg, 0.5 mmol), HOAt (75 mg, 0.55 mmol), and N,N-diisopropylethylamine (150 μL, 0.85 mmol) in DMF (4 mL) for 5 min., and then added to the above resin (0.5 mmol). The reaction mixture was agitated for 2 h, and the resin was subsequently washed with DMF (5×4 mL), followed by CH₂Cl₂ (5×4 mL). The resin was treated with trifluoroacetic acid containing 5% water and 0.5% triethylsilane for 1 h. The cleaved 2-(2-(2-(D-biotinylamino)ethoxy)ethoxy)acetic acid was purified by RP-HPLC (on a Dionex Ultimate 3000 system) using a preparative C18 column (Phenomenex Gemini, 110 Å 5 μm C18 particles, 21×100 mm): Solvent A, water containing 0.1% trifluoroacetic acid, and solvent B, acetonitrile containing 0.1% trifluoroacetic acid, were used with gradient elution (0-5 min: 5% to 100% 5-32 min) at a flow rate of 15 mL min⁻¹. This material (55 mg, 0.14 mmol) was dissolved in dry CH₂Cl₂ (5 mLQ, to which was added 4-methoxyphenol (20 mg, 0.16 mmol), 4-dimethylaminopyridine (2 mg, 0.1 mmol), followed by N,N′-diisopropylcarbodiimide (20 mg, 0.16 mmol). The reaction mixture was stirred for 16 h, after which it was concentrated by rotary evaporation. The product was purified by RP-HPLC (on a Dionex Ultimate 3000 system) using a preparative C18 column (Phenomenex Gemini, 110 Å 5 μm C18 particles, 21×100 mm): Solvent A, water containing 0.1% TFA, and solvent B, acetonitrile containing 0.1% TFA, were used with gradient elution (0-5 min: 5% to 100% 5-32 min) at a flow rate of 15 mL min⁻¹. This provided the title compound (25 mg, 36%), as a white solid. ¹H NMR (500 MHz, CD3CN), δ 7.08-7.02 (m, 2H, 2×H—Ar), 6.97-6.92 (m, 2H, 2×H—Ar), 6.50 (br s, 1H, NH), 5.15 (br s, 1H, NH), 4.95 (br s, 1H, NH), 4.40 (ddt, J=1.0 and 5.2 and 7.6 Hz, 1H, biotin CH), 4.37 (s, 2H, CH₂CO), 4.22 (ddd, J=2.0 and 4.5 and 7.6 Hz, 1H, biotin CH), 3.79 (s, 3H, OMe), 3.74-3.71 (m, 2H, CH₂O), 3.63-3.60 (m, 2H, CH₂O), 3.49 (t, J=5.4 Hz, 2H, CH₂O), 3.30 (dd, J=5.6 and 11.3 Hz, 2H, CH₂N), 3.17-3.11 (m, 1H, biotin CHS), 2.88 (dd, J=4.8 and 12.7 Hz, 1H, biotin CH₂S), 2.63 (d, J=12.7 Hz, 1H, CH₂S), 2.11 (t, partly overlapped by HDO, J=7.4 Hz, 2H, biotin CH₂CO), 1.70-1.48 (m, 4H, biotin CH₂), 1.40-1.32 (m, 2H, biotin CH₂). ¹³C NMR (125 MHz, CD₃CN), δ 173.8, 170.8, 163.7, 158.5, 144.8, 123.5, 115.5, 71.6, 70.9, 70.4, 69.1, 62.3, 60.8, 56.4, 56.3, 41.2, 39.8, 36.3, 29.0, 28.9, 26.4. HR-MS (QTOF): m/z calcd. for chemical formula C₂₃H₃₃N₃O₇₅: 495.2039; found: [M+Na]⁺ 518.1998; [M+H]⁺ 496.2124.

### 9.5 Synthesis of 3-chloro-4-methoxyphenyl 2-(2-(2-(D-biotinylamino)ethoxy)ethoxy)-acetate (6b)

0.14 mmol of 2-(2-(2-(D-biotinylamino)ethoxy)ethoxy)acetic acid (prepared as described for compound 6a) was dissolved in 5 mL of CH₂Cl₂. Thereafter, 0.16 mmol of 3-chloro-4-methoxyphenol and 0.1 mmol of 4-dimethylaminopyridine, followed by 0.16 mmol of N,N′-diisopropylcarbodiimide, was added. The reaction mixture was stirred for 16 h, after which it was concentrated by rotary evaporation. The product was purified by RP-HPLC (on a Dionex Ultimate 3000 system) using a preparative C18 column (Phenomenex Gemini, 110 Å 5 μm C18 particles, 21×100 mm): solvent A, water without any acid, and solvent B, acetonitrile without any acid, were used with gradient elution (0-5 min: 5-100% 5-27 min) at a flow rate of 15 mL min⁻¹. This provided the title compound (8 mg, 11%), as a white solid. ¹H NMR (500 MHz, CDCl₃) δ 7.19-7.17 (m, 1H; H—Ar), 7.03-6.99 (m, 1H; H—Ar), 6.94-6.91 (m, 1H; H—Ar), 6.59 (s, 1H; NH), 6.03 (s, 1H; NH), 5.16 (s, 1H; H—Ar), 4.51-4.45 (m, 1H; biotin CH), 4.38 (s, 2H; CH2CO), 4.31-4.26 (m, 1H; biotin CH), 3.90 (s, 3H; OMe), 3.82-3.77 (m, 2H; CH2O), 3.72-3.67 (m, 2H; CH2O), 3.62-3.57 (m, 2H; CH2O), 3.48-3.41 (m, 2H; CH2N), 3.15-3.07 (m, 1H; biotin CHS), 2.93-2.85 (m, 1H; biotin CH2S), 2.75-2.67 (m, 1H; biotin CH2S), 2.23-2.13 (m, 2H; biotin CH2CO), 1.77-1.56 (m, 4H; biotin CH2), 1.45-1.34 (m, 2H; biotin CH2). 13C NMR (500 MHz, CDCl3) δ 173.45 (C═O), 169.36 (C═O), 163.70 (C═O), 153.39 (C—Ar), 143.33 (C—Ar), 123.57 (C—Ar), 122.90 (C—Ar), 120.59 (C—Ar), 112.34 (C—Ar), 71.27 (CH2O), 70.16 (2×CH2O), 68.54 (CH2CO), 61.86 (biotin CH), 60.27 (biotin CH), 56.65 (CH3), 55.54 (biotin CHS), 40.68 (biotin CH2S), 39.24 (CH2N), 35.92 (biotin CH2CO), 28.19 (2×biotin CH2), 25.63 (biotin CH2). HR-MS (ESI): m/z calculated for chemical formula C₂₃H₃₂ClN₃O₇S: 529.1649; found [M+H]⁺ 530.1653.

### Example 10: Effect of pH

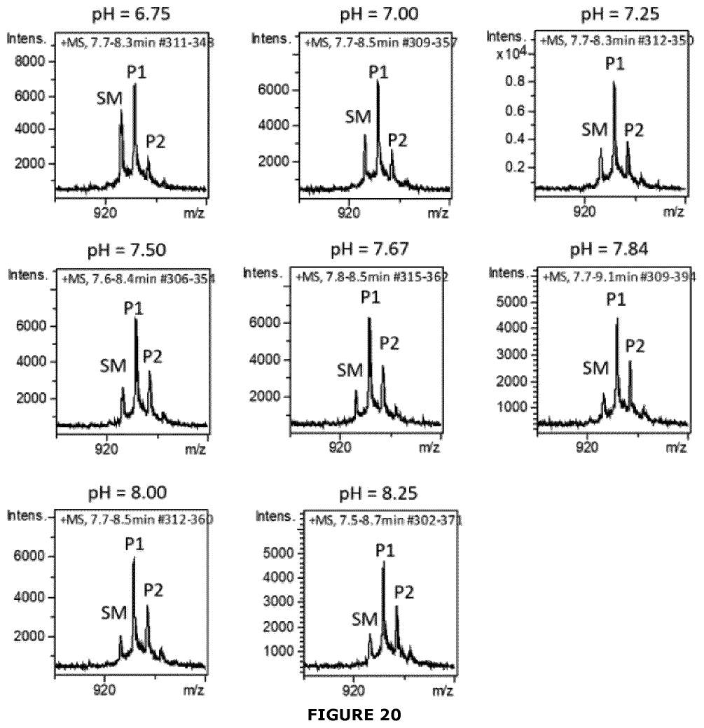

The effect of pH was studied on the conversion and selectivity of the acylation reaction, using C-terminally H₃KH₆-tagged sfGFP as model protein with acylating reagent 1. Eight different pH values between 6.75 and 8.25 were tested (FIG. 20). Below 7.25, less di-functionalized product was observed but more starting material had remained, indicating an increase in selectivity and a decrease in the reaction efficiency. Between 7.25 and 8.25 the effect of pH was less pronounced.

An advantage of the Lys-His tag acylation is the use of near-neutral pH. The pH scan in the present experiment shows that small adjustments in pH can be used to direct product formation, which may be useful when optimizing the acylation of a protein of interest.

### Example 11: Selectivity for N-s-Acylation Over N-Terminal N-α-Amine-Acylation

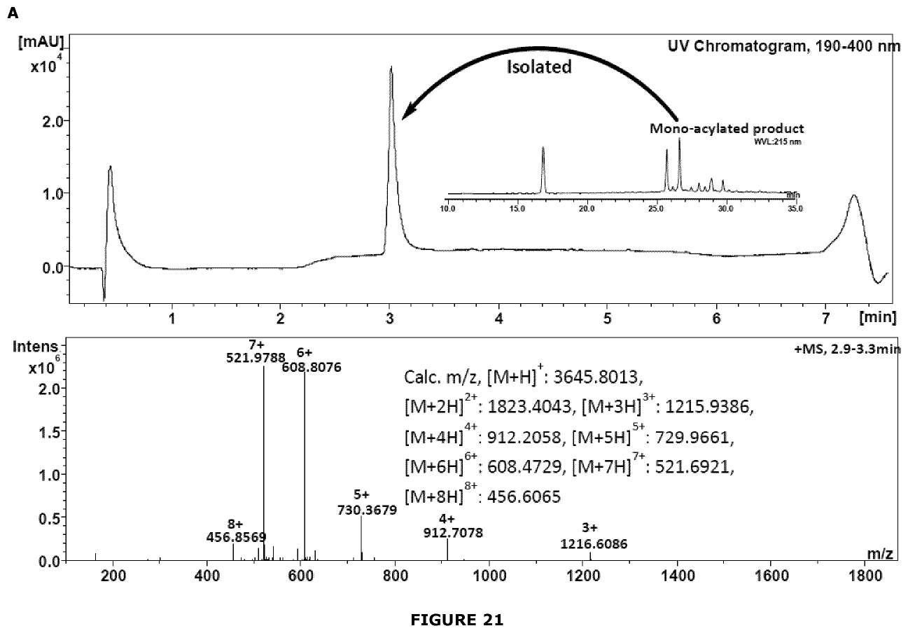

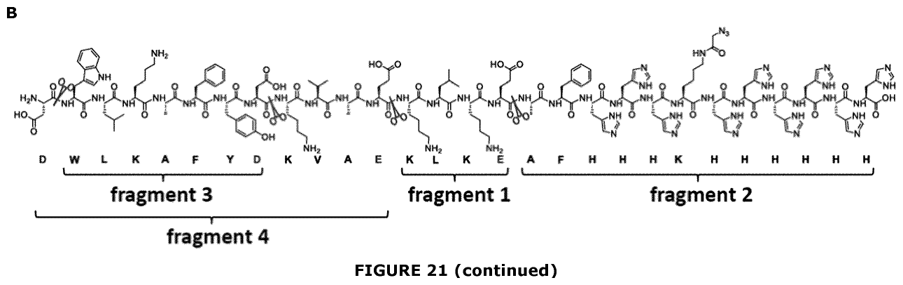

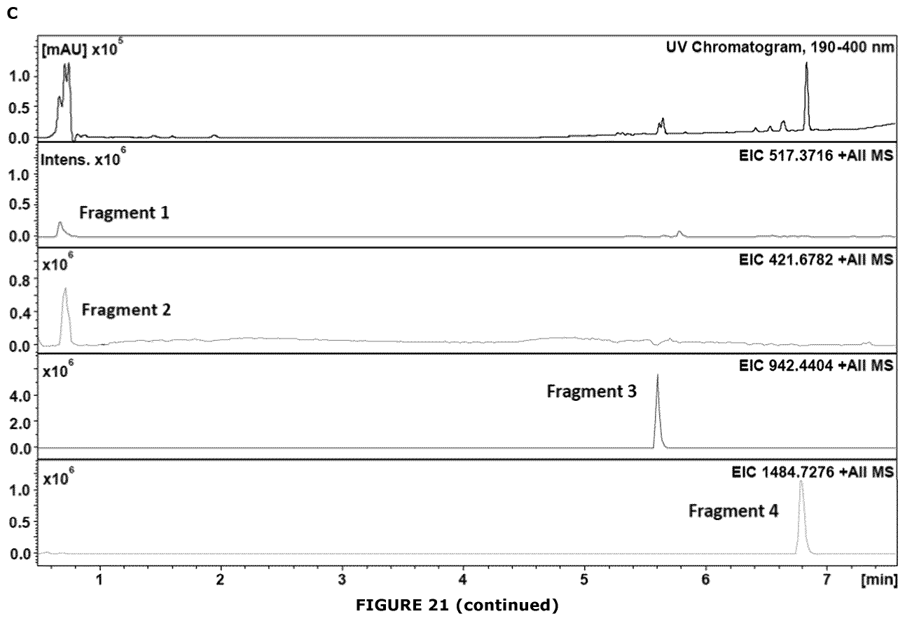

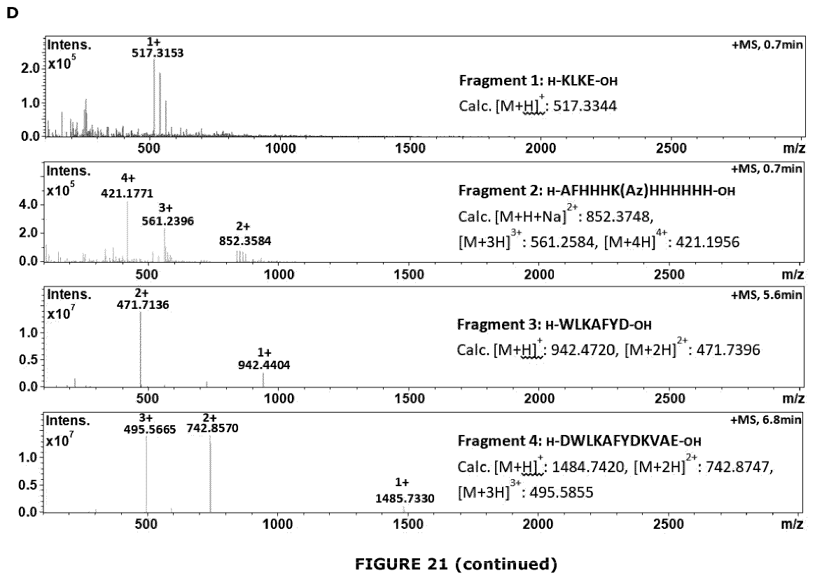

Selectivity for N-s-acylation over acylation of the N-terminal N-α-amine was assessed using the 18-mer peptide (Beltide): DWLKAFYDKVAEKLKEAF (SEQ ID NO. 69), with a free N-α-amine and C-terminal H₃KH₆ tag. The acylated Lys-His-tagged peptide was enzymatically cleaved using V8 protease, yielding four fragments of different length. The primary site of acylation was identified at the lysine in the Lys-His tag, as evidenced by LC-MS data for the fragments (FIG. 21).

### REFERENCES

1. Isom, D. G.; Castaneda, C. A.; Cannon, B. R.; Garcia-Moreno, B., Large shifts in pKa values of lysine residues buried inside a protein. Proc Natl Acad Sci USA 2011, 108 (13), 5260-5.
2. Chen, X.; Muthoosamy, K.; Pfisterer, A.; Neumann, B.; Weil, T., Site-Selective Lysine Modification of Native Proteins and Peptides via Kinetically Controlled Labeling. Bioconjugate Chem 2012, 23 (3), 500-508.
3. Matos, M. J.; Oliveira, B. L.; Martinez-Saez, N.; Guerreiro, A.; Cal, P. M. S. D.; Bertoldo, J.; Maneiro, M.; Perkins, E.; Howard, J.; Deery, M. J.; Chalker, J. M.; Corzana, F.; Jimenez-Oses, G.; Bernardes, G. J. L., Chemo- and Regioselective Lysine Modification on Native Proteins. Journal of the American Chemical Society 2018, 140 (11), 4004-4017.
4. Rosen, C. B.; Kodal, A. L. B.; Nielsen, J. S.; Schaffert, D. H.; Scavenius, C.; Okholm, A. H.; Voigt, N. V.; Enghild, J. J.; Kjems, J.; Torring, T.; Gothelf, K. V., Template-directed covalent conjugation of DNA to native antibodies, transferrin and other metal-binding proteins. Nat Chem 2014, 6 (9), 804-809.
5. Mortensen, M. R.; Skovsgaard, M. B.; Okholm, A. H.; Scavenius, C.; Dupont, D. M.; Rosen, C. B.; Enghild, J. J.; Kjems, J.; Gothelf, K. V., Small-Molecule Probes for Affinity-Guided Introduction of Biocompatible Handles on Metal-Binding Proteins. Bioconjugate Chem 2018, 29 (9), 3016-3025.
6. Bundy, B. C.; Swartz, J. R., Site-Specific Incorporation of p-Propargyloxyphenylalanine in a Cell-Free Environment for Direct Protein-Protein Click Conjugation. Bioconjugate Chem 2010, 21 (2), 255-263.
7. Lilius, G.; Persson, M.; Bulow, L.; Mosbach, K., Metal Affinity Precipitation of Proteins Carrying Genetically Attached Polyhistidine Affinity Tails. Eur J Biochem 1991, 198 (2), 499-504.
8. Carlsson, J.; Mosbach, K.; Bulow, L., Affinity precipitation and site-specific immobilization of proteins carrying polyhistidine tails. Biotechnol Bioeng 1996, 51 (2), 221-228.
9. Nielsen, M.; Lundegaard, C.; Lund, 0.; Petersen, T. N., CPHmodels-3.0—remote homology modeling using structure-guided sequence profiles. Nucleic Acids Res 2010, 38 (Web Server issue), W576-81.
10. Kelley, L. A.; Mezulis, S.; Yates, C. M.; Wass, M. N.; Sternberg, M. J., The Phyre2 web portal for protein modeling, prediction and analysis. Nat Protoc 2015, 10 (6), 845-58.
11. Waterhouse, A.; Bertoni, M.; Bienert, S.; Studer, G.; Tauriello, G.; Gumienny, R.; Heer, F. T.; de Beer, T. A. P.; Rempfer, C.; Bordoli, L.; Lepore, R.; Schwede, T., SWISS-MODEL: homology modelling of protein structures and complexes. Nucleic Acids Res 2018, 46 (W1), W296-W303.
12. Raman, S.; Vernon, R.; Thompson, J.; Tyka, M.; Sadreyev, R.; Pei, J.; Kim, D.; Kellogg, E.; DiMaio, F.; Lange, 0.; Kinch, L.; Sheffler, W.; Kim, B. H.; Das, R.; Grishin, N. V.; Baker, D., Structure prediction for CASP8 with all-atom refinement using Rosetta. Proteins 2009, 77 Suppl 9, 89-99.
13. Gasteiger, E.; Gattiker, A.; Hoogland, C.; Ivanyi, I.; Appel, R. D.; Bairoch, A., ExPASy: The proteomics server for in-depth protein knowledge and analysis. Nucleic Acids Res 2003, 31 (13), 3784-8.
14. Der, B. S.; Kluwe, C.; Miklos, A. E.; Jacak, R.; Lyskov, S.; Gray, J. J.; Georgiou, G.; Ellington, A. D.; Kuhlman, B., Alternative Computational Protocols for Supercharging Protein Surfaces for Reversible Unfolding and Retention of Stability. Plos One 2013, 8 (5).

1. A method for site-selective modification of a target protein or peptide comprising the steps of:
a. providing a target protein or peptide wherein the amino acid sequence of said protein or peptide comprises an acylation tag,
b. contacting the target protein or peptide from step (a) with an acylating reagent to form a modified target protein or peptide,
wherein said acylation tag comprises a single lysine residue and at least three histidine residues,
wherein the acylation tag is located internally or at the C-terminus of the target protein or peptide, and
wherein the target protein or peptide upon contact with the acylating reagent becomes modified at the ε-amine of the lysine residue of the acylation tag.

2. The method according to claim 1, wherein the total number of amino acids in the acylation tag is ≤25.

3. The method according to claim 1, wherein the acylation tag comprises or consists of an amino acid sequence selected from:
i.
(His)ₐ-(X¹)_{b}-Lys,
ii.
Lys-(X¹)_{b}-(His)ₐ,
iii.
(His)ₐ-(X¹)_{b}-Lys-(X²)c-(His)ₐ
and
iv.
(His)_{d}-(X²)_{c}-Lys-(X¹)_{b}-(His)ₐ
wherein a≥3, b=0-3, c=0-3, and d≥1, and
wherein X¹ and X² each are one or more identical or different amino acids but not lysine.

4. The method according to claim 1, wherein the acylating reagent is a phenyl ester derivative of the formula (I) or (II): 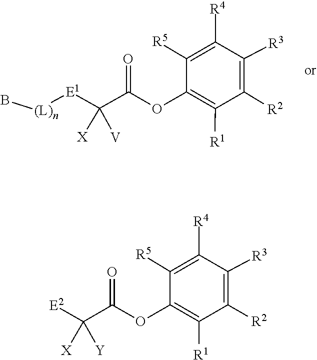
wherein E¹ and E² are an electron-withdrawing group or an alkylidene group; wherein E¹ is an attachment point of a biointeractive agent or an analytical agent; wherein E² is a reactive group that facilitates attachment to a biointeractive agent or to an analytical agent; wherein B is the biointeractive agent or analytical agent, wherein L is a linker, and n is 0 or 1; wherein the electron-withdrawing group E¹ is selected from —C(O)O—, —OC(O)—, —NHC(O)—, —C(O)NH—, —O—, —NH—, —S—, —C(O)—, —OC(O)NH—, —NHC(O)—O—, —NHC(O)—NH—, —NHC(S)—NH—, —NHS(O)₂—, —S(O)₂NH—, ═NH—O—, ═NH—NH—, ═NH—N(alkyl)-, triazole, and the electron-withdrawing group E² is selected from azide, alkyne, ring-strained alkyne, ring-strained alkene, tetrazine, nitrone, hydroxylamine, hydrazine, carbonyl, and phosphine; wherein X and Y are selected from hydrogen, an alkyl (e.g. methyl), a substituted alkyl, and an aryl group; and
wherein R¹, R², R³, R⁴, and R⁵ are selected from hydrogen, an alkyl (e.g. methyl), an alkoxy (e.g. methoxy), and a halogen (e.g. Cl or F).

5. The method according to claim 4, wherein E2 is selected from azide, a C2-C6 alkyne, a ring-strained alkyne having a ring-size of C7-C9 (e.g. DBCO, DIFO, BCN), a ring-strained alkene having a ring-size of C3-C9 (e.g. trans-cyclooctene, cyclopropene), tetrazine, nitrone, hydroxylamine, hydrazine, carbonyl, and phosphine; wherein X and Y are selected from hydrogen, a C1-C4 alkyl, and a C6-C10 aryl group; wherein R4 and R5 are hydrogen; wherein R1, R2, and R3 are selected from hydrogen, a C1-C4 alkyl, an alkoxy with a C1-C4 alkyl chain, and halogen; and wherein at least one of R1 and R3 is an electron-donating moiety.

6. The method according to claim 1, wherein the modified target protein or peptide is an acylated protein or peptide, and the method further comprises the step of attaching a biointeractive agent or an analytical agent to said modified protein or peptide.

7. The method according to claim 6, wherein the biointeractive agent or analytical agent (B) is selected from biotin, a fluorophore, a toxin, a chelator, a half-life extending moiety, an imaging reagent, a crosslinking moiety, a peptide, a protein, an oligonucleotide, a lipid, a mono- or polysaccharide, and a viral particle.

8. The method according to claim 5, wherein the reactive group (E²) that facilitates covalent attachment to a biointeractive agent or to an analytical agent is selected from azide, alkyne, ring-strained alkyne (e.g. DBCO, DIFO, BCN), ring-strained alkene (e.g. trans-cyclooctene, cyclopropene), tetrazine, nitrone, hydroxylamine, hydrazine, carbonyl, and phosphine.

9. The method according to claim 1, wherein the acylating reagent is a 4-methoxyl phenyl ester derivative of the formula (VI) or (V): 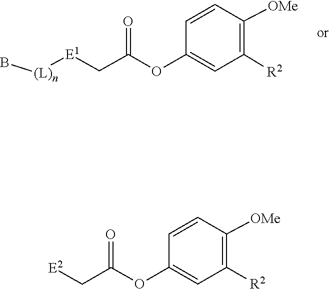
wherein B is a biointeractive agent or an analytical agent,
wherein L is a linker and n is 0 or 1,
wherein E¹ is selected from —C(O)O—, —OC(O)—, —NHC(O)—, —C(O)NH—, —O—, —NH—, —S—, —C(O)—, —OC(O)NH—, —NHC(O)—O—, —NHC(O)—NH—, —NHC(S)—NH—, —NHS(O)₂—, —S(O)₂NH—, ═NH—O—, ═NH—NH—, ═NH—N(alkyl)-, and triazole,
wherein E² is selected from azide, alkyne, ring-strained alkyne, ring-strained alkene, tetrazine, nitrone, hydroxylamine, hydrazine, carbonyl and phosphine, and wherein R² is selected from hydrogen, an alkyl (e.g. methyl), an alkoxy (e.g. methoxy), and a halogen (e.g. Cl or F).

10. An acylated protein or peptide comprising an acylation tag, wherein said acylation tag comprises or consists of an amino acid sequence selected from:
i.
(His)ₐ-(X¹)_{b}-Lys,
ii.
Lys-(X¹)_{b}-(His)ₐ,
iii.
(His)ₐ-(X¹)_{b}-Lys-(X²)c-(His)ₐ
and
iv.
(His)_{d}-(X²)_{c}-Lys-(X¹)_{b}-(His)ₐ
wherein a≥3, b=0-3, c=0-3, and d≥1, and
wherein X¹ and X² each are one or more identical or different amino acids but not lysine, and
wherein the ε-amine of Lys in said acylation tag is acylated.

11. The acylated protein or peptide according to claim 10, wherein the Lys in said acylation tag is conjugated to a biointeractive agent or an analytical agent.

12. (canceled)

13. A kit for modifying a target protein or peptide, said kit comprising
a. a target protein or peptide, or a nucleic acid sequence encoding said target protein or peptide, wherein said target protein or peptide comprises an acylation tag, wherein said acylation tag comprises or consists of an amino acid sequence selected from:
i.
(His)ₐ-(X¹)_{b}-Lys,
ii.
Lys-(X¹)_{b}-(His)ₐ,
iii.
(His)ₐ-(X¹)_{b}-Lys-(X²)c-(His)ₐ
and
iv.
(His)_{d}-(X²)_{c}-Lys-(X¹)_{b}-(His)ₐ
wherein a≥3, b=0-3, c=0-3, and d≥1, and
wherein X¹ and X² each are one or more identical or different amino acids but not lysine, and
b. an acylating reagent.

14. (canceled)

15. (canceled)


SEQUENCE LISTING (69 sequences)

SEQ ID NO: 1 - DNA, 733 bp, Artificial sequence
  synthetic
  CDS (7)..(723): Gene[sfGFP]
atatccatggcaaaaggcgaagagctgtttacgggagttgtgccgattcttgtggaactggatggtgacgtgaatggtcacaaattttccgttcgtggagaaggtgaaggtgatgcgaccaatggcaaactcacgctcaagtttatctgtacgaccgggaaactgccggtaccttggccgactttggtgacaaccctgacctatggggtccaatgctttagtcggtatccggatcacatgaaacgccatgacttcttcaaatctgcgatgcctgaagggtatgtgcaggaacgtaccatctcgttcaaagacgatggcacctataagactcgtgcggaagtaaagtttgagggcgataccctggtaaaccgcattgaactgaaaggcattgacttcaaagaggacggtaacatcttaggccacaaactggaatacaactttaacagccacaacgtctacattaccgcggataaacagaagaatggcattaaggccaacttcaaaatccgccataatgttgaagatggctcagtccagttagctgaccattaccagcagaatacgccaattggtgatggcccagttctgttacccgacaatcattatctttcgacacaaagcgtgttgagcaaagatccgaacgagaaacgcgatcatatggtgctgctggaatttgtcactgccgccggtatcacgcatggcatggatgagctgtacaaataactcgagatat

SEQ ID NO: 2 - protein, 238 aa, Artificial sequence
  Synthetic Construct
MAKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 3 - DNA, 5369 bp, Artificial sequence
  synthetic
  plasmid (1)..(5369): pET28a(+)
caccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatggctagcatgactggtggacagcaaatgggtcgcggatccgaattcgagctccgtcgacaagcttgcggccgcactcgag

SEQ ID NO: 4 - DNA, 5712 bp, Artificial sequence
  synthetic
  plasmid (1)..(5712): pNIC28-StrepTEVGlyHisSUMO
  CDS (89)..(523): SUMO
taatacgactcactataggggaattgtgagcggataacaattcccctctagaaataattttgtttaactttaagaaggagatatacatatgagcgcatggtcacatccgcagtttgaaaaaggtggtggtagcggtggtggttcaggtggtagtgcttggagccaccctcagtttgagaaagaaaatctgtattttcagggccatcatcaccatcaccacatgagcgatagcgaagttaatcaagaagcaaaaccggaagttaaacctgaagtgaaaccggaaacccatattaacctgaaagttagtgatggcagcagcgagatcttctttaaaatcaaaaaaaccacaccgctgcgtcgtctgatggaagcatttgcaaaacgtcagggtaaagaaatggatagcctgcgttttctgtatgatggtattcgtattcaggcagatcagacaccggaagatctggatatggaagataacgatattatcgaagcacatcgtgagcagattggtggtgcaacctattaaggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaat

SEQ ID NO: 5 - protein, 145 aa, Artificial sequence
  Synthetic Construct
MSAWSHPQFEKGGGSGGGSGGSAWSHPQFEKENLYFQGHHHHHHMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGATY

SEQ ID NO: 6 - DNA, 31 bp, Artificial sequence
  synthetic
  primer (1)..(31): FP-SUMO-H3KH6
atatccatgggcgatagcgaagttaatcaag

SEQ ID NO: 7 - DNA, 41 bp, Artificial sequence
  synthetic
  primer (1)..(41): RP-SUMO-H3KH6
atatggatccgcgcggcaccagataggttgcaccaccaatc

SEQ ID NO: 8 - DNA, 6910 bp, Artificial sequence
  synthetic
  plasmid (1)..(6910): pET28a(+)-4CL2-H3KH6
caccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatggagaaagatacaaaacaggttgacataattttccgatcaaaactccctgatatttacatccctaaccatcttcctttacactcctactgtttcgaaaacatttccgagttcagttctcgtccttgtttaatcaatggcgccaacaaacaaatttatacgtatgctgatgttgaactcaattcaagaaaagttgctgctggtcttcacaaacaagggattcaaccaaaggatacaataatgatcctattgcctaactccccagaatttgtgtttgctttcattggtgcatcgtacctcggagctatttctacaatggccaatcctttgtttactcctgctgaggttgtgaagcaagccgaggcttctagtgctaagatcattgtcacacaagcgtgtcatgttaacaaagtgaaagattatgcatttgagaatgatgtgaagatcatatgcatcgactcggcgccggagggttgtctccacttctccgtgctaactcaggctaatgagcacgatattcctgaggttgaaattcaacctgacgatgtggtggcgttgccatactcctccgggacgacgggattacctaaaggagtgatgttgacgcacaagggacttgtgacaagcgtcgcacaacaagtcgacggtgaaaatccgaatttgtatatccatagcgaggacgtgatgctttgtgtcttgcccttgttccatatctattcactcaactccgttttgctttgtggattaagggtgggagcagcgattttgattatgcagaaatttgatattgtttctttcttggagttgatacaaagttacaaggtgacaatagggccgtttgtaccacctattgttttggccattgctaagagtcctatggttgatgattatgatctttcatcagtaagaaccgtcatgtctggggctgcaccattaggaaaggagcttgaagatactgttcgagccaaatttcctaatgctaaacttggtcagggttatggtatgacagaagctggaccagtgttggctatgtgcttggcatttgcaaaagaaccctttgaaataaaatcaggggcatgtggaacagttgtgagaaatgctgaaatgaaaattgtggatcctaaaactggtaattctcttcccagaaatcaatctggagaaatttgcattagaggagaccagatcatgaaaggctacctgaatgatccagaggccacagcaagaacaatagacaaagaagggtggttatatactggtgacattggctacattgatgatgacgacgagcttttcattgttgatcgattaaaggaactgatcaaatacaaaggatttcaagtcgcacctgctgagctcgaagctctccttctcaaccatcccaacatttctgatgctgctgttgtccccatgaaggacgagcaagcaggagaagttccagtggcttttgttgttagatccaacggatccaccattactgaagatgaagtcaaagattttatttcaaagcaggtgatattttataagaggataaagcgggtatttttcgtggatgctattcctaaatctccatctggcaaaatccttcgaaaagatttgagagctaaactggctgctgggcttccaaatctggtgccgcgcggatcccatcaccataaacaccatcaccaccatcattaactcgag

SEQ ID NO: 9 - DNA, 20 bp, Artificial sequence
  synthetic
  primer (1)..(20): T7 primer
taatacgactcactataggg

SEQ ID NO: 10 - DNA, 44 bp, Artificial sequence
  synthetic
  primer (1)..(44): RP-sfGFP-H3KH6
atatggatccgcgcggcaccagttatttgtacagctcatccatg

SEQ ID NO: 11 - DNA, 7030 bp, Artificial sequence
  synthetic
  plasmid (1)..(7030): pET28a(+)-MBPstar-TEV[54-237]
  CDS (5071)..(6255): MBP
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatggcaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaggacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaaccccatgagcgagaatctttattttcagggcgccagtggatcctagaccctgctggttcagagcctgcatggtgtttttaaagtgaaaaataccaccaccctgcagcagcatctgattgatggtcgtgatatgattattatccgcatgccgaaagattttccgccttttccgcagaaactgaaatttcgtgaaccgcagcgtgaagaacgtatttgtctggttaccaccaattttcagaccaaaagcatgagcagcatggttagcgataccagctgtacctttccgagcagtgatggcattttttggaaacattggattcagacgaaagatggtcagtgtggtagtccgctggttagcacccgtgatggttttattgttggtattcatagcgccagcaatttcaccaacaccaacaactattttaccagcgtgccgaaaaactttatggaactgctgaccaatcaagaggcacagcagtgggttagcggttggcgtctgaatgcagatagcgttctgtggggtggtcataaagtgtttatggttaaaccggaagaaccgtttcagccggttaaagaagcaacccagctgatgaatgaactggtgccgcgcggcagcggcagcagccatcatcatcatcatcattaagcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 12 - protein, 395 aa, Artificial sequence
  Synthetic Construct
MAIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNPMSENLYFQGASGS

SEQ ID NO: 13 - DNA, 27 bp, Artificial sequence
  synthetic
  primer (1)..(27): FP-MBP-H3KH6
atatcaccccatggcaatcgaagaagg

SEQ ID NO: 14 - DNA, 35 bp, Artificial sequence
  synthetic
  primer (1)..(35): RP-MBP-H3KH6
atatggatccactggcgccctgaaaataaagattc

SEQ ID NO: 15 - DNA, 30 bp, Artificial sequence
  synthetic
  primer (1)..(30): Forward DNA oligo KH6 (phosphorylated 5′ end)
gatccaaacaccatcaccaccatcattaac

SEQ ID NO: 16 - DNA, 30 bp, Artificial sequence
  synthetic
  primer (1)..(30): Reverse DNA oligo KH6 (phosphorylated 5′ end)
tcgagttaatgatggtggtgatggtgtttg

SEQ ID NO: 17 - DNA, 769 bp, Artificial sequence
  synthetic
  CDS (7)..(759): Gene[sfGFP(H6)]
atatccatggcaaaaggcgaagagctgtttacgggagttgtgccgattcttgtggaactggatggtgacgtgaatggtcacaaattttccgttcgtggagaaggtgaaggtgatgcgaccaatggcaaactcacgctcaagtttatctgtacgaccgggaaactgccggtaccttggccgactttggtgacaaccctgacctatggggtccaatgctttagtcggtatccggatcacatgaaacgccatgacttcttcaaatctgcgatgcctgaagggtatgtgcaggaacgtaccatctcgttcaaagacgatggcacctataagactcgtgcggaagtaaagtttgagggcgataccctggtaaaccgcattgaactgaaaggcattgacttcaaagaggacggtaacatcttaggccacaaactggaatacaactttaacagccacaacgtctacattaccgcggataaacagaagaatggcattaaggccaacttcaaaatccgccataatgttgaagatgcggccgcacaccatcaccaccatcatgccgctgcaggctcagtccagttagctgaccattaccagcagaatacgccaattggtgatggcccagttctgttacccgacaatcattatctttcgacacaaagcgtgttgagcaaagatccgaacgagaaacgcgatcatatggtgctgctggaatttgtcactgccgccggtatcacgcatggcatggatgagctgtacaaataactcgagatat

SEQ ID NO: 18 - protein, 250 aa, Artificial sequence
  Synthetic Construct
MAKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDAAAHHHHHHAAAGSVQLADHYQQNTPIGDGPVLLPDNHYLSTQSVLSKDPNEKRDHMVLLEFVTAAGITHGMDELYK

SEQ ID NO: 19 - DNA, 769 bp, Artificial sequence
  synthetic
  CDS (7)..(759): Gene[sfGFP(KH6)]
atatccatggcaaaaggcgaagagctgtttacgggagttgtgccgattcttgtggaactggatggtgacgtgaatggtcacaaattttccgttcgtggagaaggtgaaggtgatgcgaccaatggcaaactcacgctcaagtttatctgtacgaccgggaaactgccggtaccttggccgactttggtgacaaccctgacctatggggtccaatgctttagtcggtatccggatcacatgaaacgccatgacttcttcaaatctgcgatgcctgaagggtatgtgcaggaacgtaccatctcgttcaaagacgatggcacctataagactcgtgcggaagtaaagtttgagggcgataccctggtaaaccgcattgaactgaaaggcattgacttcaaagaggacggtaacatcttaggccacaaactggaatacaactttaacagccacaacgtctacattaccgcggataaacagaagaatggcattaaggccaacttcaaaatccgccataatgttgaagatgccgcgaaacaccatcaccaccatcatgcagctgccggttcagtccagttagctgaccattaccagcagaatacgccaattggtgatggcccagttctgttacccgacaatcattatctttcgacacaaagcgtgttgagcaaagatccgaacgagaaacgcgatcatatggtgctgctggaatttgtcactgcagccggtatcacgcatggcatggatgagctgtacaaataaggatccatat

SEQ ID NO: 20 - protein, 250 aa, Artificial sequence
  Synthetic Construct
MAKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDAAKHHHHHHAAAGSVQLADHYQQNTPIGDGPVLLPDNHYLSTQSVLSKDPNEKRDHMVLLEFVTAAGITHGMDELYK

SEQ ID NO: 21 - DNA, 5708 bp, Artificial sequence
  synthetic
  plasmid (1)..(5708): pET15b
ggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatgctcgaggatcc

SEQ ID NO: 22 - DNA, 769 bp, Artificial sequence
  synthetic
  CDS (7)..(759): Gene[sfGFP(H6K)]
atatccatggcaaaaggcgaagagctgtttacgggagttgtgccgattcttgtggaactggatggtgacgtgaatggtcacaaattttccgttcgtggagaaggtgaaggtgatgcgaccaatggcaaactcacgctcaagtttatctgtacgaccgggaaactgccggtaccttggccgactttggtgacaaccctgacctatggggtccaatgctttagtcggtatccggatcacatgaaacgccatgacttcttcaaatctgcgatgcctgaagggtatgtgcaggaacgtaccatctcgttcaaagacgatggcacctataagactcgtgcggaagtaaagtttgagggcgataccctggtaaaccgcattgaactgaaaggcattgacttcaaagaggacggtaacatcttaggccacaaactggaatacaactttaacagccacaacgtctacattaccgcggataaacagaagaatggcattaaggccaacttcaaaatccgccataatgttgaagatgccgcggcacaccatcaccaccatcataaagctgccggttcagtccagttagctgaccattaccagcagaatacgccaattggtgatggcccagttctgttacccgacaatcattatctttcgacacaaagcgtgttgagcaaagatccgaacgagaaacgcgatcatatggtgctgctggaatttgtcactgcagccggtatcacgcatggcatggatgagctgtacaaataaggatccatat

SEQ ID NO: 23 - protein, 250 aa, Artificial sequence
  Synthetic Construct
MAKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDAAAHHHHHHKAAGSVQLADHYQQNTPIGDGPVLLPDNHYLSTQSVLSKDPNEKRDHMVLLEFVTAAGITHGMDELYK

SEQ ID NO: 24 - DNA, 48 bp, Artificial sequence
  synthetic
  primer (1)..(48): DNA oligo T3A
atatgcggccgcacaccatcacaaacaccatcatgccgctgcagatat

SEQ ID NO: 25 - DNA, 48 bp, Artificial sequence
  synthetic
  primer (1)..(48): DNA oligo T3B
atatctgcagcggcatgatggtgtttgtgatggtgtgcggccgcatat

SEQ ID NO: 26 - DNA, 50 bp, Artificial sequence
  synthetic
  primer (1)..(50): DNA oligo T5A
tatgcggccgcagagaaacaccatcaccaccatcatgccgctgcagatat

SEQ ID NO: 27 - DNA, 50 bp, Artificial sequence
  synthetic
  primer (1)..(50): DNA oligo T5B
atatctgcagcggcatgatggtggtgatggtgtttctctgcggccgcata

SEQ ID NO: 28 - DNA, 50 bp, Artificial sequence
  synthetic
  primer (1)..(50): DNA oligo T6A
tatgcggccgcacaccatcacgagaaacaccatcatgccgctgcagatat

SEQ ID NO: 29 - DNA, 50 bp, Artificial sequence
  synthetic
  primer (1)..(50): DNA oligo T6B
atatctgcagcggcatgatggtgtttctcgtgatggtgtgcggccgcata

SEQ ID NO: 30 - DNA, 57 bp, Artificial sequence
  synthetic
  primer (1)..(57): DNA oligo T7A
atatgcggccgcacatcaccataaacaccatcaccaccatcatgccgctgcagatat

SEQ ID NO: 31 - DNA, 57 bp, Artificial sequence
  synthetic
  primer (1)..(57): DNA oligo T7B
atatctgcagcggcatgatggtggtgatggtgtttatggtgatgtgcggccgcatat

SEQ ID NO: 32 - DNA, 50 bp, Artificial sequence
  synthetic
  primer (1)..(50): DNA oligo T8A
tatgcggccgcacaccatcaccctaaacaccatcatgccgctgcagatat

SEQ ID NO: 33 - DNA, 50 bp, Artificial sequence
  synthetic
  primer (1)..(50): DNA oligo T8B
atatctgcagcggcatgatggtgtttagggtgatggtgtgcggccgcata

SEQ ID NO: 34 - DNA, 345 bp, Artificial sequence
  synthetic
  CDS (1)..(345): SUMO-KH6
atgggcgatagcgaagttaatcaagaagcaaaaccggaagttaaacctgaagtgaaaccggaaacccatattaacctgaaagttagtgatggcagcagcgagatcttctttaaaatcaaaaaaaccacaccgctgcgtcgtctgatggaagcatttgcaaaacgtcagggtaaagaaatggatagcctgcgttttctgtatgatggtattcgtattcaggcagatcagacaccggaagatctggatatggaagataacgatattatcgaagcacatcgtgagcagattggtggtgcaacctatctggtgccgcgcggatccaaacaccatcaccaccatcattaa

SEQ ID NO: 35 - protein, 114 aa, Artificial sequence
  Synthetic Construct
MGDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGATYLVPRGSKHHHHHH

SEQ ID NO: 36 - DNA, 354 bp, Artificial sequence
  synthetic
  CDS (1)..(354): SUMO-H3KH6
atgggcgatagcgaagttaatcaagaagcaaaaccggaagttaaacctgaagtgaaaccggaaacccatattaacctgaaagttagtgatggcagcagcgagatcttctttaaaatcaaaaaaaccacaccgctgcgtcgtctgatggaagcatttgcaaaacgtcagggtaaagaaatggatagcctgcgttttctgtatgatggtattcgtattcaggcagatcagacaccggaagatctggatatggaagataacgatattatcgaagcacatcgtgagcagattggtggtgcaacctatctggtgccgcgcggatcccatcaccataaacaccatcaccaccatcattaa

SEQ ID NO: 37 - protein, 117 aa, Artificial sequence
  Synthetic Construct
MGDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGATYLVPRGSHHHKHHHHHH

SEQ ID NO: 38 - DNA, 756 bp, Artificial sequence
  synthetic
  CDS (1)..(756): sfGFP-KH6
atggcaaaaggcgaagagctgtttacgggagttgtgccgattcttgtggaactggatggtgacgtgaatggtcacaaattttccgttcgtggagaaggtgaaggtgatgcgaccaatggcaaactcacgctcaagtttatctgtacgaccgggaaactgccggtaccttggccgactttggtgacaaccctgacctatggggtccaatgctttagtcggtatccggatcacatgaaacgccatgacttcttcaaatctgcgatgcctgaagggtatgtgcaggaacgtaccatctcgttcaaagacgatggcacctataagactcgtgcggaagtaaagtttgagggcgataccctggtaaaccgcattgaactgaaaggcattgacttcaaagaggacggtaacatcttaggccacaaactggaatacaactttaacagccacaacgtctacattaccgcggataaacagaagaatggcattaaggccaacttcaaaatccgccataatgttgaagatggctcagtccagttagctgaccattaccagcagaatacgccaattggtgatggcccagttctgttacccgacaatcattatctttcgacacaaagcgtgttgagcaaagatccgaacgagaaacgcgatcatatggtgctgctggaatttgtcactgccgccggtatcacgcatggcatggatgagctgtacaaactggtgccgcgcggatccaaacaccatcaccaccatcattaa

SEQ ID NO: 39 - protein, 251 aa, Artificial sequence
  Synthetic Construct
MAKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKLVPRGSKHHHHHH

SEQ ID NO: 40 - DNA, 765 bp, Artificial sequence
  synthetic
  CDS (1)..(765): sfGFP-H3KH6
atggcaaaaggcgaagagctgtttacgggagttgtgccgattcttgtggaactggatggtgacgtgaatggtcacaaattttccgttcgtggagaaggtgaaggtgatgcgaccaatggcaaactcacgctcaagtttatctgtacgaccgggaaactgccggtaccttggccgactttggtgacaaccctgacctatggggtccaatgctttagtcggtatccggatcacatgaaacgccatgacttcttcaaatctgcgatgcctgaagggtatgtgcaggaacgtaccatctcgttcaaagacgatggcacctataagactcgtgcggaagtaaagtttgagggcgataccctggtaaaccgcattgaactgaaaggcattgacttcaaagaggacggtaacatcttaggccacaaactggaatacaactttaacagccacaacgtctacattaccgcggataaacagaagaatggcattaaggccaacttcaaaatccgccataatgttgaagatggctcagtccagttagctgaccattaccagcagaatacgccaattggtgatggcccagttctgttacccgacaatcattatctttcgacacaaagcgtgttgagcaaagatccgaacgagaaacgcgatcatatggtgctgctggaatttgtcactgccgccggtatcacgcatggcatggatgagctgtacaaactggtgccgcgcggatcccatcaccataaacaccatcaccaccatcattaa

SEQ ID NO: 41 - protein, 254 aa, Artificial sequence
  Synthetic Construct
MAKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKLVPRGSHHHKHHHHHH

SEQ ID NO: 42 - DNA, 1209 bp, Artificial sequence
  synthetic
  CDS (1)..(1209): MBP-KH6
atggcaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaggacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaaccccatgagcgagaatctttattttcagggcgccagtggatccaaacaccatcaccaccatcattaa

SEQ ID NO: 43 - protein, 402 aa, Artificial sequence
  Synthetic Construct
MAIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNPMSENLYFQGASGSKHHHHHH

SEQ ID NO: 44 - DNA, 1218 bp, Artificial sequence
  synthetic
  CDS (1)..(1218): MBP-H3KH6
atggcaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaggacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaaccccatgagcgagaatctttattttcagggcgccagtggatcccatcaccataaacaccatcaccaccatcattaa

SEQ ID NO: 45 - protein, 405 aa, Artificial sequence
  Synthetic Construct
MAIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNPMSENLYFQGASGSHHHKHHHHHH

SEQ ID NO: 46 - DNA, 759 bp, Artificial sequence
  synthetic
  CDS (1)..(759): sfGFP(EKH6)
atggcaaaaggcgaagagctgtttacgggagttgtgccgattcttgtggaactggatggtgacgtgaatggtcacaaattttccgttcgtggagaaggtgaaggtgatgcgaccaatggcaaactcacgctcaagtttatctgtacgaccgggaaactgccggtaccttggccgactttggtgacaaccctgacctatggggtccaatgctttagtcggtatccggatcacatgaaacgccatgacttcttcaaatctgcgatgcctgaagggtatgtgcaggaacgtaccatctcgttcaaagacgatggcacctataagactcgtgcggaagtaaagtttgagggcgataccctggtaaaccgcattgaactgaaaggcattgacttcaaagaggacggtaacatcttaggccacaaactggaatacaactttaacagccacaacgtctacattaccgcggataaacagaagaatggcattaaggccaacttcaaaatccgccataatgttgaagatgcggccgcagagaaacaccatcaccaccatcatgccgctgcaggctcagtccagttagctgaccattaccagcagaatacgccaattggtgatggcccagttctgttacccgacaatcattatctttcgacacaaagcgtgttgagcaaagatccgaacgagaaacgcgatcatatggtgctgctggaatttgtcactgccgccggtatcacgcatggcatggatgagctgtacaaataa

SEQ ID NO: 47 - protein, 252 aa, Artificial sequence
  Synthetic Construct
MAKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDAAAEKHHHHHHAAAGSVQLADHYQQNTPIGDGPVLLPDNHYLSTQSVLSKDPNEKRDHMVLLEFVTAAGITHGMDELYK

SEQ ID NO: 48 - DNA, 756 bp, Artificial sequence
  synthetic
  CDS (1)..(756): sfGFP(H3KH3)
atggcaaaaggcgaagagctgtttacgggagttgtgccgattcttgtggaactggatggtgacgtgaatggtcacaaattttccgttcgtggagaaggtgaaggtgatgcgaccaatggcaaactcacgctcaagtttatctgtacgaccgggaaactgccggtaccttggccgactttggtgacaaccctgacctatggggtccaatgctttagtcggtatccggatcacatgaaacgccatgacttcttcaaatctgcgatgcctgaagggtatgtgcaggaacgtaccatctcgttcaaagacgatggcacctataagactcgtgcggaagtaaagtttgagggcgataccctggtaaaccgcattgaactgaaaggcattgacttcaaagaggacggtaacatcttaggccacaaactggaatacaactttaacagccacaacgtctacattaccgcggataaacagaagaatggcattaaggccaacttcaaaatccgccataatgttgaagatgcggccgcacaccatcacaaacaccatcatgccgctgcaggctcagtccagttagctgaccattaccagcagaatacgccaattggtgatggcccagttctgttacccgacaatcattatctttcgacacaaagcgtgttgagcaaagatccgaacgagaaacgcgatcatatggtgctgctggaatttgtcactgccgccggtatcacgcatggcatggatgagctgtacaaataa

SEQ ID NO: 49 - protein, 251 aa, Artificial sequence
  Synthetic Construct
MAKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDAAAHHHKHHHAAAGSVQLADHYQQNTPIGDGPVLLPDNHYLSTQSVLSKDPNEKRDHMVLLEFVTAAGITHGMDELYK

SEQ ID NO: 50 - DNA, 759 bp, Artificial sequence
  synthetic
  CDS (1)..(759): sfGFP(H3EKH3)
atggcaaaaggcgaagagctgtttacgggagttgtgccgattcttgtggaactggatggtgacgtgaatggtcacaaattttccgttcgtggagaaggtgaaggtgatgcgaccaatggcaaactcacgctcaagtttatctgtacgaccgggaaactgccggtaccttggccgactttggtgacaaccctgacctatggggtccaatgctttagtcggtatccggatcacatgaaacgccatgacttcttcaaatctgcgatgcctgaagggtatgtgcaggaacgtaccatctcgttcaaagacgatggcacctataagactcgtgcggaagtaaagtttgagggcgataccctggtaaaccgcattgaactgaaaggcattgacttcaaagaggacggtaacatcttaggccacaaactggaatacaactttaacagccacaacgtctacattaccgcggataaacagaagaatggcattaaggccaacttcaaaatccgccataatgttgaagatgcggccgcacaccatcacgagaaacaccatcatgccgctgcaggctcagtccagttagctgaccattaccagcagaatacgccaattggtgatggcccagttctgttacccgacaatcattatctttcgacacaaagcgtgttgagcaaagatccgaacgagaaacgcgatcatatggtgctgctggaatttgtcactgccgccggtatcacgcatggcatggatgagctgtacaaataa

SEQ ID NO: 51 - protein, 252 aa, Artificial sequence
  Synthetic Construct
MAKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDAAAHHHEKHHHAAAGSVQLADHYQQNTPIGDGPVLLPDNHYLSTQSVLSKDPNEKRDHMVLLEFVTAAGITHGMDELYK

SEQ ID NO: 52 - DNA, 759 bp, Artificial sequence
  synthetic
  CDS (1)..(759): sfGFP(H3PKH3)
atggcaaaaggcgaagagctgtttacgggagttgtgccgattcttgtggaactggatggtgacgtgaatggtcacaaattttccgttcgtggagaaggtgaaggtgatgcgaccaatggcaaactcacgctcaagtttatctgtacgaccgggaaactgccggtaccttggccgactttggtgacaaccctgacctatggggtccaatgctttagtcggtatccggatcacatgaaacgccatgacttcttcaaatctgcgatgcctgaagggtatgtgcaggaacgtaccatctcgttcaaagacgatggcacctataagactcgtgcggaagtaaagtttgagggcgataccctggtaaaccgcattgaactgaaaggcattgacttcaaagaggacggtaacatcttaggccacaaactggaatacaactttaacagccacaacgtctacattaccgcggataaacagaagaatggcattaaggccaacttcaaaatccgccataatgttgaagatgcggccgcacaccatcaccctaaacaccatcatgccgctgcaggctcagtccagttagctgaccattaccagcagaatacgccaattggtgatggcccagttctgttacccgacaatcattatctttcgacacaaagcgtgttgagcaaagatccgaacgagaaacgcgatcatatggtgctgctggaatttgtcactgccgccggtatcacgcatggcatggatgagctgtacaaataa

SEQ ID NO: 53 - protein, 252 aa, Artificial sequence
  Synthetic Construct
MAKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDAAAHHHPKHHHAAAGSVQLADHYQQNTPIGDGPVLLPDNHYLSTQSVLSKDPNEKRDHMVLLEFVTAAGITHGMDELYK

SEQ ID NO: 54 - DNA, 765 bp, Artificial sequence
  synthetic
  CDS (1)..(765): sfGFP(H3KH6)
atggcaaaaggcgaagagctgtttacgggagttgtgccgattcttgtggaactggatggtgacgtgaatggtcacaaattttccgttcgtggagaaggtgaaggtgatgcgaccaatggcaaactcacgctcaagtttatctgtacgaccgggaaactgccggtaccttggccgactttggtgacaaccctgacctatggggtccaatgctttagtcggtatccggatcacatgaaacgccatgacttcttcaaatctgcgatgcctgaagggtatgtgcaggaacgtaccatctcgttcaaagacgatggcacctataagactcgtgcggaagtaaagtttgagggcgataccctggtaaaccgcattgaactgaaaggcattgacttcaaagaggacggtaacatcttaggccacaaactggaatacaactttaacagccacaacgtctacattaccgcggataaacagaagaatggcattaaggccaacttcaaaatccgccataatgttgaagatgcggccgcacatcaccataaacaccatcaccaccatcatgccgctgcaggctcagtccagttagctgaccattaccagcagaatacgccaattggtgatggcccagttctgttacccgacaatcattatctttcgacacaaagcgtgttgagcaaagatccgaacgagaaacgcgatcatatggtgctgctggaatttgtcactgccgccggtatcacgcatggcatggatgagctgtacaaataa

SEQ ID NO: 55 - protein, 254 aa, Artificial sequence
  Synthetic Construct
MAKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDAAAHHHKHHHHHHAAAGSVQLADHYQQNTPIGDGPVLLPDNHYLSTQSVLSKDPNEKRDHMVLLEFVTAAGITHGMDELYK

SEQ ID NO: 56 - DNA, 6683 bp, Artificial sequence
  synthetic
  plasmid (1)..(6683): Expression vector HC-KH6-Rituximab (heavy chain tagged with KH6)
  CDS (691)..(2121): Rutuximab heavy chain tagged with KH6
  CDS (4482)..(5180): Rutuximab light chain
gcgcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgcattcctgcaggtcgacaccatgggctggtcctgcatcatcctgtttctggtggctaccgccaccggcgtgcactctcaggtgcagctgcagcagcctggcgccgagctcgtgaaacctggcgcctccgtgaagatgtcctgcaaggcctccggctacaccttcaccagctacaacatgcactgggtcaagcagacccccggcagaggcctggaatggatcggcgctatctaccccggcaacggcgacacctcctacaaccagaagttcaagggcaaggccaccctgaccgccgacaagtcctcttccaccgcctacatgcagctgtcctccctgacctccgaggactccgccgtgtactactgcgcccggtctacctactacggcggcgactggtacttcaacgtgtggggcgctggcaccaccgtgaccgtgtctgctgcttctaccaagggcccctccgtgttccctctggccccttccagcaagtctacctctggcggcacagccgctctgggctgcctcgtgaaggactacttccccgagcccgtgacagtgtcctggaactctggcgctctgaccagcggagtgcacaccttccctgctgtgctgcagtcctccggcctgtactccctgtccagcgtcgtgactgtgccctccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggacccagcgtgttcctgttccccccaaagcccaaggataccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacatgcctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactatacccagaagtccctgtccctgagccccggcaagcaccatcatcaccatcactgaggatccgaacaaaaactcatctcagaagaggatctgaatatgcataccggtcatcatcaccatcaccattgagtttgatccccgggaattcagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtttagttcctcaccttgtcgtattatactatgccgatatactatgccgatgattaattgtcaacacgtgctgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctcgtcgagctagcttcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctcgttggaatttgccctttttgagtttggatcttggttcattctcaagcctcagacagtggttcaaagtttttttcttccatttcaggtgtcgtgaacacgtggtcgcggccgcaccatgggctggtcctgcatcatcctgtttctggtggctaccgccaccggcgtgcactcccagattgtgctgtctcagtcccccgccatcctgtctgctagccctggcgagaaagtgacaatgacctgccgggcctcctcctccgtgtcctacatccactggttccagcagaagcccggctccagccccaagccttggatctacgccacctccaacctggcctctggcgtgccagtgcggttttccggctctggctctggcacctcctactccctgaccatctctcgggtggaagccgaggatgccgccacctactactgccagcagtggaccagcaacccccccacatttggcggaggcaccaagctggaaatcaagcggaccgtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctacagcctgtcctccaccctgaccctgtccaaggccgactacgagaagcacaaggtgtacgcctgcgaagtgacccaccagggcctgtctagccccgtgaccaagtctttcaaccggggcgagtgctgactcgagagatctggccggctgggcccgtttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggcgccattcgccattcaggctgcgcaactgttgggaagggcgatcggtgcgggcctcttcgctattacgcca

SEQ ID NO: 57 - protein, 476 aa, Artificial sequence
  Synthetic Construct
MGWSCIILFLVATATGVHSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 58 - protein, 232 aa, Artificial sequence
  Synthetic Construct
MGWSCIILFLVATATGVHSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - DNA, 6692 bp, Artificial sequence
  synthetic
  plasmid (1)..(6692): Expression vector HC-H3KH6-Rituximab (heavy chain tagged with H3KH6)
  CDS (691)..(2130): Rituximab heavy chain (tagged with H3KH6)
  CDS (4491)..(5189): Rituximab light chain
gcgcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgcattcctgcaggtcgacaccatgggctggtcctgcatcatcctgtttctggtggctaccgccaccggcgtgcactctcaggtgcagctgcagcagcctggcgccgagctcgtgaaacctggcgcctccgtgaagatgtcctgcaaggcctccggctacaccttcaccagctacaacatgcactgggtcaagcagacccccggcagaggcctggaatggatcggcgctatctaccccggcaacggcgacacctcctacaaccagaagttcaagggcaaggccaccctgaccgccgacaagtcctcttccaccgcctacatgcagctgtcctccctgacctccgaggactccgccgtgtactactgcgcccggtctacctactacggcggcgactggtacttcaacgtgtggggcgctggcaccaccgtgaccgtgtctgctgcttctaccaagggcccctccgtgttccctctggccccttccagcaagtctacctctggcggcacagccgctctgggctgcctcgtgaaggactacttccccgagcccgtgacagtgtcctggaactctggcgctctgaccagcggagtgcacaccttccctgctgtgctgcagtcctccggcctgtactccctgtccagcgtcgtgactgtgccctccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggacccagcgtgttcctgttccccccaaagcccaaggataccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacatgcctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactatacccagaagtccctgtccctgagccccggccaccatcataagcaccatcatcaccatcactgaggatccgaacaaaaactcatctcagaagaggatctgaatatgcataccggtcatcatcaccatcaccattgagtttgatccccgggaattcagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtttagttcctcaccttgtcgtattatactatgccgatatactatgccgatgattaattgtcaacacgtgctgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctcgtcgagctagcttcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctcgttggaatttgccctttttgagtttggatcttggttcattctcaagcctcagacagtggttcaaagtttttttcttccatttcaggtgtcgtgaacacgtggtcgcggccgcaccatgggctggtcctgcatcatcctgtttctggtggctaccgccaccggcgtgcactcccagattgtgctgtctcagtcccccgccatcctgtctgctagccctggcgagaaagtgacaatgacctgccgggcctcctcctccgtgtcctacatccactggttccagcagaagcccggctccagccccaagccttggatctacgccacctccaacctggcctctggcgtgccagtgcggttttccggctctggctctggcacctcctactccctgaccatctctcgggtggaagccgaggatgccgccacctactactgccagcagtggaccagcaacccccccacatttggcggaggcaccaagctggaaatcaagcggaccgtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctacagcctgtcctccaccctgaccctgtccaaggccgactacgagaagcacaaggtgtacgcctgcgaagtgacccaccagggcctgtctagccccgtgaccaagtctttcaaccggggcgagtgctgactcgagagatctggccggctgggcccgtttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggcgccattcgccattcaggctgcgcaactgttgggaagggcgatcggtgcgggcctcttcgctattacgcca

SEQ ID NO: 60 - protein, 479 aa, Artificial sequence
  Synthetic Construct
MGWSCIILFLVATATGVHSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGHHHKHHHHHH

SEQ ID NO: 61 - protein, 232 aa, Artificial sequence; identical to SEQ ID NO: 58
  Synthetic Construct

SEQ ID NO: 62 - protein, 7 aa, Artificial sequence
  synthetic
  peptide (1)..(7): peptide tag KHHHHHH (KH6)
KHHHHHH

SEQ ID NO: 63 - protein, 10 aa, Artificial sequence
  synthetic
  peptide (1)..(10): peptide tag HHHKHHHHHH (H3KH6)
HHHKHHHHHH

SEQ ID NO: 64 - protein, 7 aa, Artificial sequence
  synthetic
  peptide (1)..(7): peptide tag HHHKHHH (H3KH3)
HHHKHHH

SEQ ID NO: 65 - protein, 7 aa, Artificial sequence
  synthetic
  peptide (1)..(7): peptide tag HHHHHHK (H6K)
HHHHHHK

SEQ ID NO: 66 - protein, 8 aa, Artificial sequence
  synthetic
  peptide (1)..(8): peptide tag EKHHHHHH (EKH6)
EKHHHHHH

SEQ ID NO: 67 - protein, 8 aa, Artificial sequence
  synthetic
  peptide (1)..(8): peptide tag HHHEKHHH (H3EKH3)
HHHEKHHH

SEQ ID NO: 68 - protein, 8 aa, Artificial sequence
  synthetic
  peptide (1)..(8): peptide tag HHHPKHHH (H3PKH6)
HHHPKHHH

SEQ ID NO: 69 - protein, 18 aa, Artificial sequence
  synthetic
  PEPTIDE (1)..(18): Beltide peptide
DWLKAFYDKVAEKLKEAF